(19) United States
(12) Patent Application Publication
(10) Pub. No.: US 2024/0182472 A1
(43) Pub. Date: Jun. 6, 2024

(54) INDAZOLE PYRIDONE COMPOUNDS AND USES THEREOF

(71) Applicant: Bluejay Therapeutics, Inc., San Mateo, CA (US)

(72) Inventors: Jeffery Zablocki, San Mateo, CA (US); Hassan Javanbakht, San Mateo, CA (US); Keting Chu, San Mateo, CA (US); Dmitry Koltun, San Mateo, CA (US); Nancy Shulman, San Mateo, CA (US)

(21) Appl. No.: 18/480,499

(22) Filed: Oct. 3, 2023

Related U.S. Application Data
(60) Provisional application No. 63/378,354, filed on Oct. 4, 2022.

(51) Int. Cl. C07D 471/14; A61P 31/20

(52) U.S. Cl. CPC C07D 471/14; A61P 31/20

(57) ABSTRACT

Novel tetracyclic pyridone compounds according to Formula I are provided,

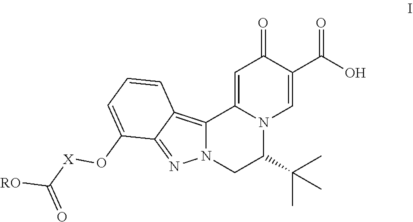

where R and X are defined herein. Pharmaceutical compositions containing such compounds, methods of using these compounds and compositions in the treatment and prevention of HBV infections are also provided.

### FIELD OF THE INVENTION

The present invention relates to novel fused tetracyclic pyridone compounds that are inhibitors of hepatitis virus expression inhibitor acting selectively in the liver, and are thus useful to treat viral infections, and particularly hepatitis B virus (HBV) and hepatitis D virus (HDV). The invention provides novel tetracyclic pyridone compounds as disclosed herein, pharmaceutical compositions containing such compounds, and methods of using these compounds and compositions in the treatment and prevention of HBV infections.

### BACKGROUND

Hepatitis B virus (HBV) infection is one of the most common infectious diseases in the world. Chronic hepatitis B (CHB) represents a critical unmet medical need with over 240 million people chronically infected worldwide. Chronic HBV carriers can develop serious liver diseases such as chronic hepatitis, cirrhosis, and primary hepatocellular carcinoma (HCC). Approximately, 650,000 people die every year due to the consequences of CHB [(Chisari, lsogawa et al. 2010, 2016),(G. B. D. Mortality Causes of Death Collaborators 2016, World Health Organization (WHO) 2018)].

The goal of CHB therapy is to improve quality of life and survival by preventing the progression of the disease to cirrhosis and HCC. HBV functional cure, also known as loss of HBV surface protein (HBsAg), with or without seroconversion to anti-HBsAg, is the accepted endpoint for anti-HBV therapy.(Lok and McMahon 2009, EASL 2012, Sarin, Kumar et al. 2015, Terrault, Bzowej et al. 2016, European Association for the Study of the Liver 2017). Multiple previous studies have shown that loss of HBsAg has been associated with improvements in liver histology, including the reversal of cirrhosis, a decreased risk of HCC, and prolonged survival, and is considered evidence of a functional cure (Fattovich, Giustina et al. 1998, Benias and Min 2011, Kim, Lim et al. 2013).

Nucleos(t)ide analogs are the standard-of-care for CHB treatment that suppress viral replication and results in long-term clinical benefits with a reduced risk of liver complications (Dienstag, Goldin et al. 2003, Liaw 2011, Lok 2013). However, treatment with nucleos(t)ide inhibitors rarely results in functional cure (Kwon and Lok 2011). Thus, new treatment options that enhance rates of HBsAg clearance are needed to provide a finite-duration treatment option for a functional cure.

Clearance of HBV-infected hepatocytes requires a broad immune response against HBV (Rehermann and Nascimbeni 2005, Das and Maini 2010, Burton, Pallett et al. 2018). Previous studies suggest that in chronic HBV infection, high levels of dominant viral antigens such as HBsAg may contribute to exhaustion of antiviral CD8+T-cells (Frebel, Richter et al. 2010, lsogawa, Chung et al. 2013, Ochel, Cebula et al. 2016, Zhu, Liu et al. 2016). In addition, several other reports describe that HBsAg negatively regulates HBV-specific immune responses by direct modulation of dendritic cells (DC), monocytes and natural killer cells (NK) functions (Chen, Wei et al. 2005, Op den Brouw, Binda et al. 2009, Woltman, Op den Brouw et al. 2011, Kondo, Ninomiya et al. 2013, Mueller, Wildum et al. 2017). Furthermore, a preclinical study has shown that reduction of extracellular HBsAg with a monoclonal HBsAg antibody followed by vaccination can clear HBV in both serum and liver in a mouse model of chronic HBV(Zhu, Liu et al. 2016).

Altogether, these studies suggest a potential therapeutic role for antiviral agents such as PAPDS/7 inhibitors (HBV RNA destabilizers/degraders/HBsAg secretion inhibitors) to reduce HBsAg levels and restore virus-specific immune responsiveness in CHB.

The hepatitis delta virus (HDV) is the causative agent of chronic hepatitis delta (CHD), the most severe form of viral hepatitis. At least 12 million individuals are HBV/HDV-coinfected worldwide, but the global number may be underestimated due to suboptimal testing. Infection with HDV can occur either simultaneously with HBV, or as superinfection in patients already chronically infected with HBV. The dependency of HDV on HBV is primarily due to using HBV-encoded envelope proteins (HBsAg) for its release and de novo infection. Efficient antiviral treatments are urgently needed to prevent its progressive course leading to the development of cirrhosis, end-stage liver disease and hepatocellular carcinoma infection. (Dandri, Volmari et al. 2022).

The minimum acceptable endpoint for new anti-HDV therapy is a greater than or equal to 2-log₁₀ decline in HDV RNA and alanine transferase (ALT) normalization on-treatment (FDA 2019, Yurdaydin, Abbas et al. 2019). A previous study demonstrated that a 2-log₁₀ decline in HDV RNA in patients treated with conventional interferon was associated with the survival benefit in CHD (Farci, Roskams et al. 2004).

Pegylated interferon alpha (pegIFNα) has been used as an off-label treatment against HDV, despite associated side effects, low response rates and high relapse rates (Bahcecioglu, Ispiroglu et al. 2015, Rizzetto and Smedile 2015). Among the new anti-HDV strategies, bulevirtide can efficiently blocks HBV and HDV entry by targeting host receptor NTCP and is the first HDV-specific drug that obtained conditional marketing authorization in Europe (Masetti and Aghemo 2021, Lampertico, Roulot et al. 2022). Lonafarnib, currently being evaluated in clinical trials, is an inhibitor of the farnesyl transferase and thus inhibits HDV release (Yurdaydin, Keskin et al. 2022). Most recently, a preclinical study demonstrated that anti-HBsAg monoclonal antibodies neutralize HDV in vitro and significantly reduce HDV RNA levels in a mouse model of CHD (Lempp 2021).

Several previous clinical studies evaluated the intravenous or subcutaneous administration of anti-HBsAg monoclonal antibodies in CHB patients and shown them to be safe and well-tolerated and resulted in significant declines in peripheral HBsAg levels in nearly all subjects (Galun, Eren et al. 2002, Lee, Park et al. 2020, Agarwal, Yuen et al. 2022).

Together, these data demonstrate the potential role of lowering HBsAg in developing anti-HBV anti HDV therapies. We describe herein our novel liver targeted PAPD5/7 inhibitors (HBV RNA destabilizers/degraders/HBsAg secretion inhibitors) designed to lower HBsAg in patients with HBV or HBV/HDV that alone or in combination with other Anti-HBV/Anti-HBD agents can lead to functional cure.

A recent study demonstrated the neurotoxicity of a hepatitis B virus (HBV) PAPD5/7 inhibitors (HBV RNA destabilizers/degraders/HBsAg secretion inhibitors), specifically, GS-8873 that is closely related structurally to the first inhibitor of the class of compounds R07834, wherein GS-8873 differs by the replacement of carbon by nitrogen to form a hydrazine based core [Lake, April D. et al Toxicological Sciences (2022) 186(2), P. 298-308 structures shown below].

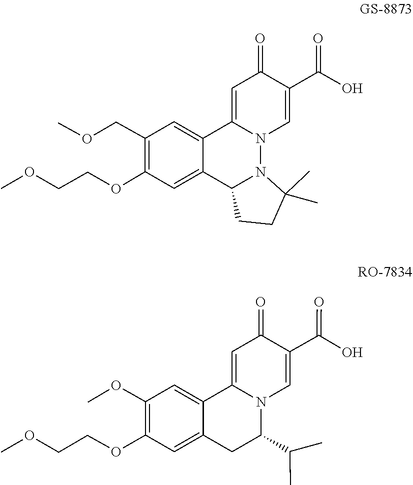 [SMILES: COCCOc1cc2c(cc1COC)-c1cc(=O)c(C(=O)O)cn1N1[C@@H]2CCC1(C)C ; COCCOc1cc2c(cc1OC)-c1cc(=O)c(C(=O)O)cn1[C@H](C(C)C)C2]

GS-8873 was found to prolong the nerve conduction velocity (NCV) of several peripheral nerves in both rats and monkeys starting at 4 weeks with a more pronounced effect at 13 weeks. The rat was found to be the more sensitive species where rat-Caudal nerve NCV, digital NCV, cauda equina latency, digital latency, tibial nerve latency were all changed by greater than 20% change from baseline at 13 weeks following daily doses of GS-8873 at 20 MPK/day and 60 MPK/day. These changes are thought to corroborate peripheral neuropathy that occurs in monkeys in these 13 week studies. In addition, GS-8873 had significant effects on rat functional tests starting at 4 weeks. It is not known whether this peripheral CNS toxicology is driven by a chemotype specific tox or a on-target based tox. Several compounds from this class have entered clinical trials only to be withdrawn or studies stopped (Roche RO7834, Enanta EDP-721) where the reasons for stopping have not been fully disclosed. Thus, the precedence for systemic safety signals with this class of HBV RNA destabilizers has caused us to focus our efforts on liver targeting mechanisms that can provide elevated levels of drug in the liver with low systemic drug exposures to avoid systemic based safety signals.

Although ACC inhibitors have the potential to address pathogenic contributors to NASH, the importance of de novo lipogenesis within human bone marrow for platelet production limits the degree of systemic ACC inhibition safely tolerated during chronic treatment. Pfizer and Gilead-Nimbus ACC inhibitors were designed with the incorporation of structural features intended for recognition by organic anion transporting polypeptides (OATPs), members of the solute carrier organic anion (SLCO) superfamily of xenobiotic transporters. Of the 11 human OATP transporters, OATP1B1 and OATP1B3 are expressed on the sinusoidal membrane of hepatocytes and can facilitate the liver uptake of their respective substrates (e.g., statins such as atorvastatin and rosuvastatin Kalliokoski, A.; et al Br. J. Pharmacol. 2009, 158, 693-705). Pfizer and Gilead-Nimbus sought ACC inhibitors that were OATP1B1/1B3 transport substrates to drive hepato-selectivity. Thus, precedence of both ACC inhibitors and statins to utilize liver uptake by OATP1B1 and OATP1B3 to drive hepato-selectivity bodes well for engaging these same transporters for HBV RNA destabilizers.

### SUMMARY OF THE INVENTION

A first aspect of the present invention is a compound of Formula (I):

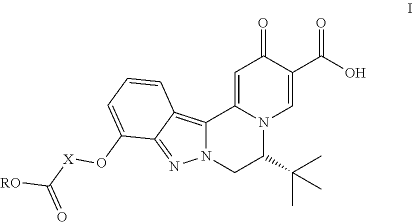

or a pharmaceutically acceptable salt thereof, wherein:
R is selected from hydrogen, ethyl, or linear, cyclic, or branched C₃-C₈ alkyl groups and X is a linker selected from linear, cyclic, or branched C₅-C₁₀ alkylene groups; racemic mixtures thereof; and pharmaceutical compositions thereof.

A second aspect of the present invention is a method of treating a patient with a viral infection, such as chronic Hepatitis B or D infections, by administering an effective amount of a pharmaceutical composition containing a compound of Formula (I):

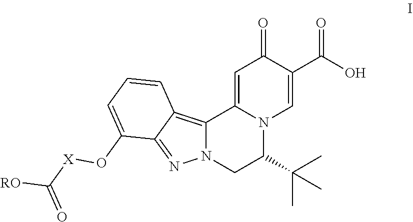

or a pharmaceutically acceptable salt thereof, wherein:
R is selected from hydrogen, ethyl, or linear, cyclic, or branched C₃-C₈ alkyl groups and X is a linker selected from linear, cyclic, or branched C₅-C₁₀ alkylene groups, or a racemic mixture thereof.

A third aspect of the present invention is a method of preventing a patient from developing downstream liver diseases resulting from chronic Hepatitis B or D infections by administering an effective amount of a pharmaceutical composition containing a compound of Formula (I):

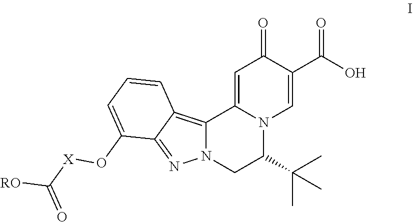

or a pharmaceutically acceptable salt thereof, wherein:
R is selected from hydrogen, ethyl, or linear, cyclic, or branched C₃-C₈ alkyl groups and X is a linker selected from linear, cyclic, or branched C₅-C₁₀ alkylene groups, or a racemic mixture thereof.

A fourth aspect of the present invention is the use of a compound of Formula (I):

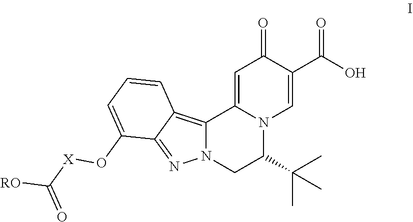

or a pharmaceutically acceptable salt thereof, wherein:
R is selected from hydrogen, ethyl, or linear, cyclic, or branched C₃-C₈ alkyl groups and X is a linker selected from linear, cyclic, or branched C₅-C₁₀ alkylene groups, or a racemic mixture thereof, in the manufacture of a medicament for the treatment of a patient with a viral infection, such as Hepatitis B or D infections.

A fifth aspect of the present invention is the use of a compound of Formula (I):

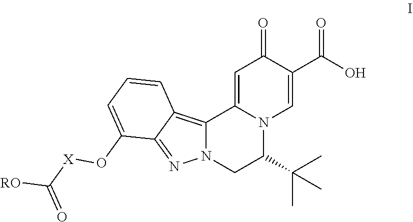

wherein:
R is selected from hydrogen, ethyl, or linear, cyclic, or branched C₃-C₈ alkyl groups and X is a linker selected from linear, cyclic, or branched C₅-C₁₀ alkylene groups, or a racemic mixture thereof, in the manufacture of a medicament for the prevention of a patient from developing downstream liver diseases resulting from chronic Hepatitis B or D infections.

A sixth aspect of the present invention is a method of treating a patient with a viral infection by administering an effective amount of a pharmaceutical composition containing a compound of Formula (I):

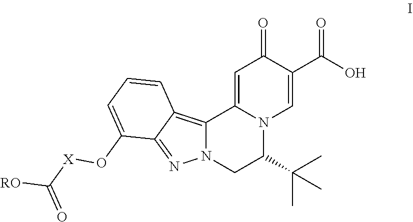

or a pharmaceutically acceptable salt thereof, wherein:
R is selected from hydrogen, ethyl, or linear, cyclic, or branched C₃-C₈ alkyl groups and X is a linker selected from linear, cyclic, or branched C₅-C₁₀ alkylene groups, or a racemic mixture thereof; the method further comprising: administering to the subject an additional therapeutic agent selected from the group consisting of HBV replication inhibitors including but not limited to a nucleos(t)ide analogue polymerase inhibitor, a non-nucleos(t)ide analogue polymerase inhibitors; HBsAg targeting agents including but not limited to siRNA and antisense targeting HBV transcript(s), HBV capsid inhibitor, cccDNA inhibitor, HBx inhibitor and an antibody targeting HBV proteins; lmmunomodulator including but not limited to an immune checkpoint inhibitor (a small molecule inhibitor or a blockade antibody), a native or modified cytokine—interferon-alpha, a TLR agonist—TLR-7, TLR-9, and TLR-8, and an immunomodulator vaccine.

A seventh aspect of the present invention is a method of preventing a patient from developing downstream liver diseases resulting from chronic Hepatitis B or D infections by administering an effective amount of a pharmaceutical composition containing a compound of Formula (I):

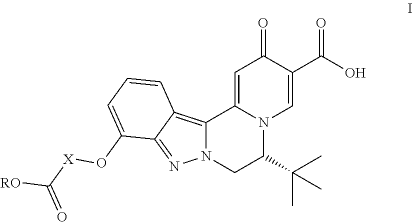

or a pharmaceutically acceptable salt thereof, wherein:
R is selected from hydrogen, ethyl, or linear, cyclic, or branched C₃-C₈ alkyl groups and X is a linker selected from linear, cyclic, or branched C₅-C₁₀ alkylene groups or a racemic mixture thereof; further comprising: administering to the subject an additional therapeutic agent selected from the group consisting of HBV replication inhibitors including but not limited to a nucleos(t)ide analogue polymerase inhibitor, a non-nucleos(t)ide analogue polymerase inhibitors; HBsAg targeting agents including but not limited to siRNA and antisense targeting HBV transcript(s), HBV capsid inhibitor, cccDNA inhibitor, HBx inhibitor and an antibody targeting HBV proteins; immunomodulator including but not limited to an immune checkpoint inhibitor (a small molecule inhibitor or a blockade antibody), a native or modified cytokine—interferon-alpha, a TLR agonist—TLR-7, TLR-9, and TLR-8, and an immunomodulator vaccine.

In some embodiments of the compound of Formula (I), X is a linear C₅-C₈ alkylene group.

In some embodiments of the compound of Formula (I), X is selected from the group consisting of a linear —C₅H₁₀—, linear —C₆H₁₂—, linear —C₇H₁₄—, and linear —C₈H₁₆.

In some embodiments of the compound of Formula (I), R is selected from the group consisting of n-C₂H₅, n-C₃H₇, i-C₃H₇, n-C₄H₉, n-C₅H₁₁, n-C₆H₁₃, 2-ethylbutyl, n-C₇H₁₅, and n-C₈H₁₇.

In some embodiments of the compound of Formula (I), R is H.

In some embodiments of the compound of Formula (I), R is i-C₃H₇.

In some embodiments of the compound of Formula (I), R is n-C₄H₉.

In some embodiments, the compound of Formula (I) is one of the following:

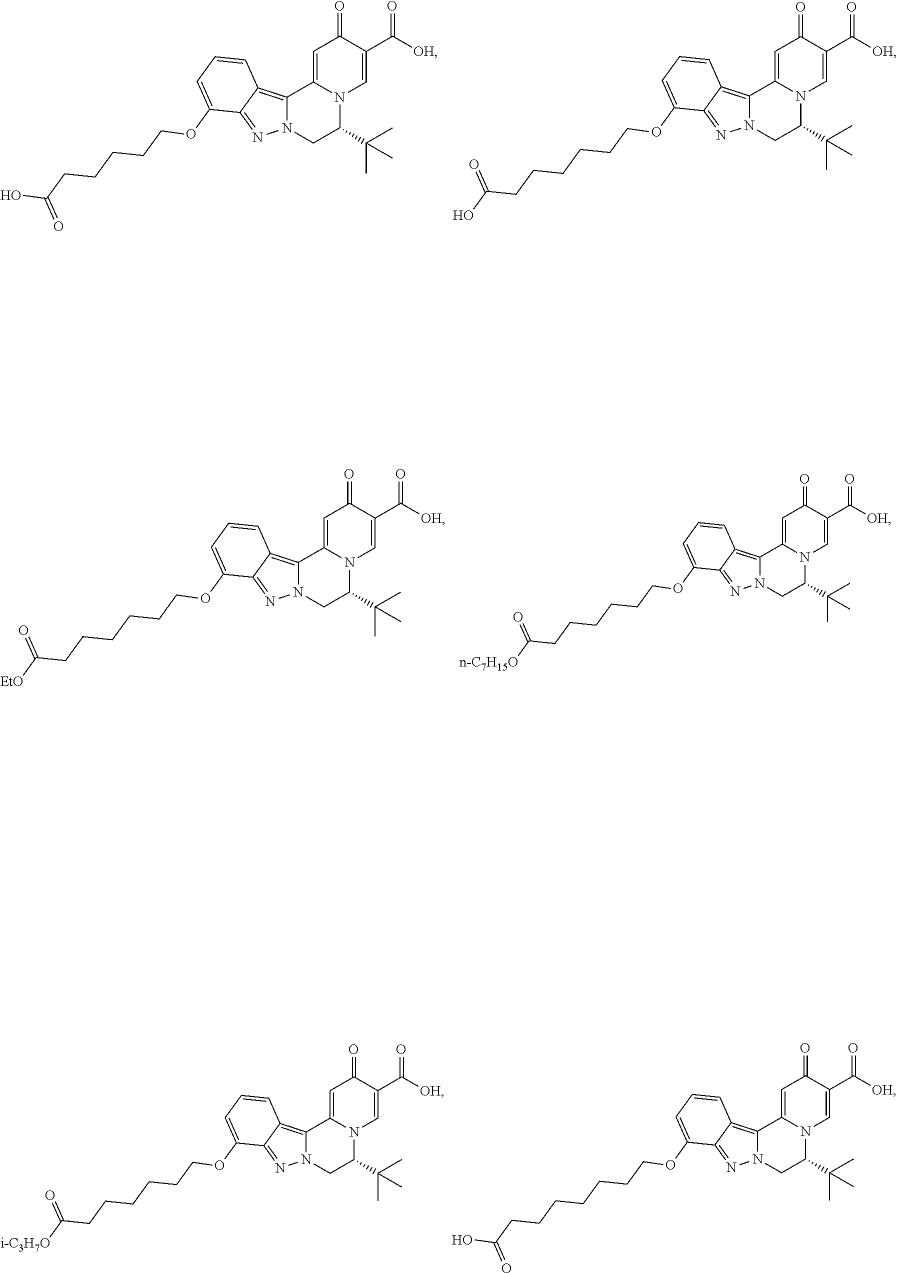

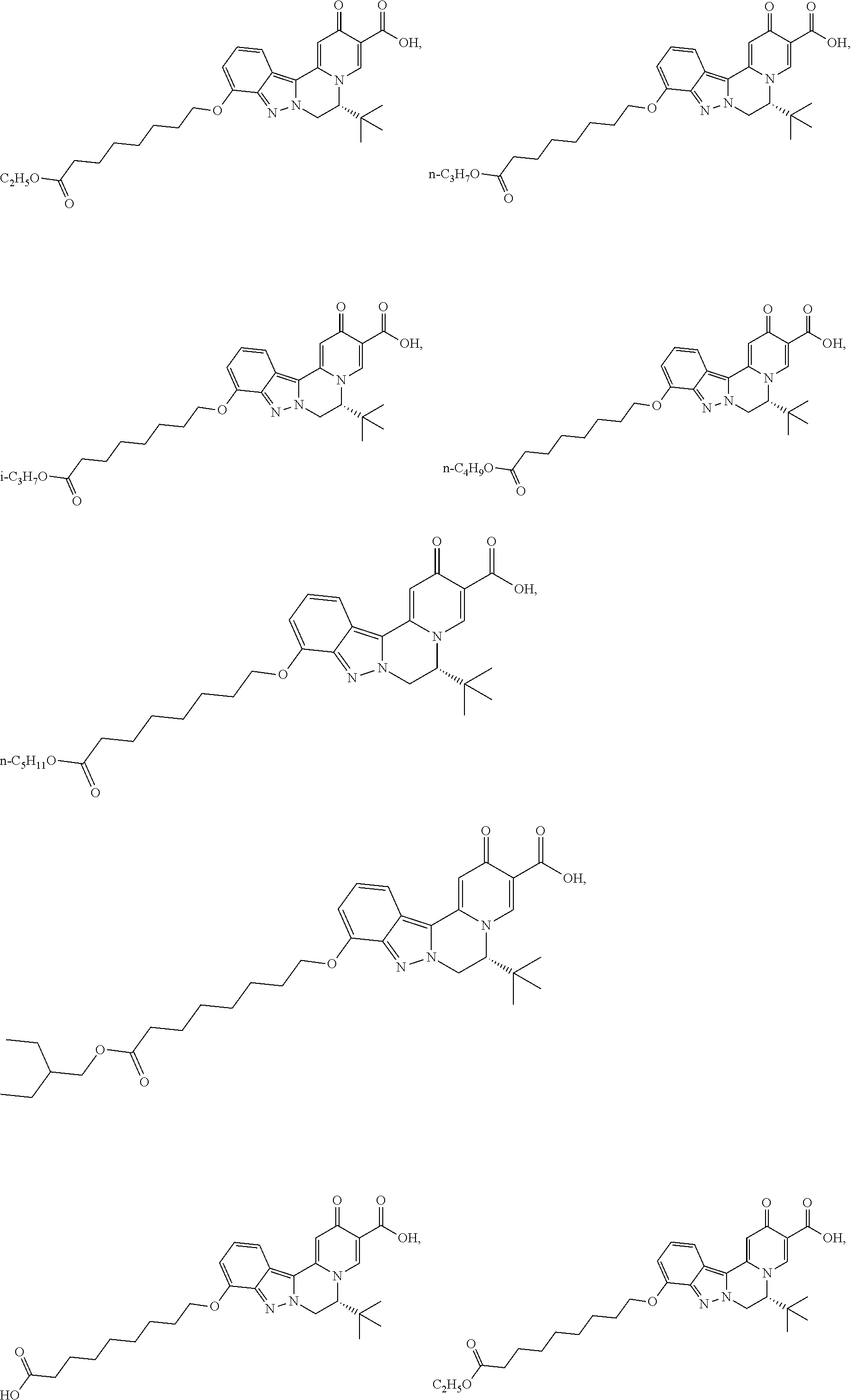

### BRIEF DESCRIPTION OF THE DRAWINGS

FIG. 1 Efficacy of Certain Compounds of Formula I at lowering HBV S-antigen level when dosed in an in vivo AAV mouse model.

### DETAILED DESCRIPTION OF THE INVENTION

The present invention provides novel compounds that inhibit secretion of HBsAg from cells infected with hepatitis B virus and thereby reduce viral load and viral replication in patients having chronic HBV infection. Thus, the compounds of the invention are suitable for treatment of patients with HBV, including chronic HBV. The compounds of the invention are also suitable for treatment of patients with HBD, including those co-infected with HBV.

Each of the compounds of the Examples, including each compound listed in Table 1, is a specific embodiment of the compounds of the invention. The following descriptions of preferred embodiments of the present invention are not intended to limit the scope of the invention.

In some embodiments, a compound of Formula I below is provided:

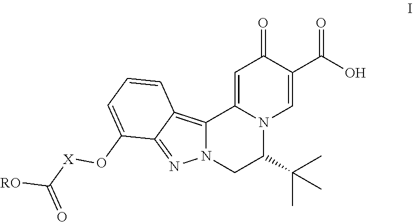

wherein R is hydrogen, ethyl, or a linear, cyclic, or branched C₃-C₈ alkyl group, and X is a linear, cyclic, or branched C₅-C₈ alkylene group. Pharmaceutically acceptable salts thereof, racemic mixtures thereof, and pharmaceutical compositions thereof are also included.

Preferred is a compound of Formula I wherein X is selected from the group consisting of a linear —C₅H₁₀—, linear —C₆H₁₂—, and linear —C₇H₁₄—; pharmaceutically acceptable salts thereof; and pharmaceutical compositions thereof.

Preferred is a compound of Formula I wherein R is selected from the group consisting of n-C₂H₅, n-C₃H₇, i-C₃H₇, n-C₄H₉, n-C₅H₁₁, n-C₆H₁₃, 2-ethylbutyl, n-C₇H₁₅, and n-C₈H₁₇; pharmaceutically acceptable salts thereof; racemic mixtures thereof; and pharmaceutical compositions thereof.

Preferred is a compound of Formula I wherein R is H; pharmaceutically acceptable salts thereof; racemic mixtures thereof; and pharmaceutical compositions thereof.

Preferred is a compound of Formula I wherein R is i-C₃H₇; pharmaceutically acceptable salts thereof; racemic mixtures thereof; and pharmaceutical compositions thereof.

Preferred is a compound of Formula I wherein R is n-C₄H₉; pharmaceutically acceptable salts thereof; racemic mixtures thereof; and pharmaceutical compositions thereof.

Preferred are the compounds of Formula (I) exemplified in the following Table 1; pharmaceutically acceptable salts thereof; racemic mixtures thereof; and pharmaceutical compositions thereof.

Preferred are the compound I.6, I.9, or I.10 exemplified in the following Table 1; a pharmaceutically acceptable salt thereof; racemic mixtures thereof; and pharmaceutical compositions thereof.

Preferred is a method of treating a patient with chronic HBV infection by administering a therapeutic amount of a pharmaceutical composition containing a compound of Formula (I) or a racemic mixture thereof; preferably a compound of Formula (I) wherein R is selected from the group consisting of n-C₂H₅, n-C₃H₇, n-C₄H₉, n-C₅H₁₁, n-C₆H₁₃, 2-ethylbutyl, n-C₇H₁₅, and n-C₈H₁₇, and preferably wherein R is H, i-C₃H₇ or n-C₄H₉; and X is a linear C₅-C₈ alkylene group, preferably linear —C₅H₁₀—, linear —C₆H₁₂—, and linear —C₇H₁₄—; and preferably wherein the compound of Formula (I) is a compound exemplified in the following Table 1; preferably the compound I.6, I.9, or I.10 exemplified in the following Table 1.

Preferred is the use of a compound of Formula (I) or a pharmaceutically acceptable salt thereof, in the manufacture of a medicament for the treatment of a patient with HBV infection; preferably a compound of Formula (I) wherein R is selected from the group consisting of n-C₂H₅, n-C₃H₇, n-C₄H₉, n-C₅H₁₁, n-C₆H₁₃, 2-ethylbutyl, n-C₇H₁₅, and n-C₈H₁₇, and preferably wherein R is H, i-C₃H₇ or n-C₄H₉; and X is a linear C₅-C₈ alkylene group, preferably linear —C₅H₁₀—, linear —C₆H₁₂—, and linear —C₇H₁₄—; and preferably wherein the compound of Formula (1) is a compound exemplified in the following Table 1; preferably the compound I.6, I.9, or I.10 exemplified in the following Table 1.

Preferred is a method of treating a patient with chronic HBV infection by administering an effective amount of a compound of Formula (I) or a pharmaceutical composition comprising an effective amount of a compound of Formula (I), or pharmaceutically acceptable salt thereof; preferably a compound of Formula (I) wherein R is selected from the group consisting of n-C₂H₅, n-C₃H₇, n-C₄H₉, n-C₅H₁₁, n-C₆H₁₃, 2-ethylbutyl, n-C₇H₁₅, and n-C₈H₁₇, and preferably wherein R is H, i-C₃H₇ or n-C₄H₉; and X is a linear C₅-C₈ alkylene group, preferably linear —C₅H₁₀—, linear —C₆H₁₂—, and linear —C₇H₁₄—; and preferably wherein the compound of Formula (I) is a compound exemplified in the following Table 1; preferably the compound I.6, I.9, or I.10 exemplified in the following Table 1; further comprising: administering to the subject an additional therapeutic agent selected from the group consisting of HBV replication inhibitors including but not limited to a nucleos(t)ide analogue polymerase inhibitor, a non-nucleos(t)ide analogue polymerase inhibitors; HBsAg targeting agents including but not limited to siRNA and antisense targeting HBV transcript(s), HBV capsid inhibitor, cccDNA inhibitor, HBx inhibitor and an antibody targeting HBV proteins; Immunomodulator including but not limited to an immune checkpoint inhibitor (a small molecule inhibitor or a blockade antibody), a native or modified cytokine—interferon-alpha, a TLR agonist TLR-7, TLR-9, and TLR-8, and an immunomodulator vaccine.

While one enantiomer of compounds of this formula is typically more active than the other enantiomer, both isomers exhibit activity on HBsAg as demonstrated in published PCT applications WO 2018/198079 and U.S. Pat. No. 10,301,312 B2 Date of Patent: May 28, 2019 whose contents are incorporated entirely by this reference.

The term “an optical isomer” or “a stereoisomer” refers to any of the various stereoisomeric configurations which may exist for a given compound of the present invention and includes geometric isomers. It is understood that a substituent may be attached at a chiral center of a carbon atom. The term “chiral” refers to molecules which have the property of non-superimposable on their mirror image partner, while the term “achiral” refers to molecules which are superimposable on their mirror image partner. Therefore, the invention includes enantiomers, diastereomers or racemates of the compound. “Enantiomers” are a pair of stereoisomers that are non-superimposable mirror images of each other. A 1:1 mixture of a pair of enantiomers is a “racemic” mixture. The term is used to designate a racemic mixture where appropriate. “Diastereoisomers” are stereoisomers that have at least two asymmetric atoms, but which are not mirror-images of each other. The absolute stereochemistry is specified according to the Cahn-Ingold-Prelog R-S system. When a compound is a pure enantiomer the stereochemistry at each chiral carbon may be specified by either R or S.

Resolved compounds whose absolute configuration is unknown can be designated (+) or (−) depending on the direction (dextro- or levorotatory) which they rotate plane polarized light at the wavelength of the sodium D line. Certain compounds described herein contain one or more asymmetric centers or axes and may thus give rise to enantiomers, diastereomers, and other stereoisomeric forms that may be defined, in terms of absolute stereochemistry, as (R)- or (S)-.

As used herein, “prodrugs” are molecules that are converted to active parent drug in vivo often addressing physical properties of the active parent that are unfavorable for high oral bioavailability. The “prodrugs” have a built-in structural lability, whether by chance or by design, that permits bioconversion in vivo to the active parent drug. This conversion can occur through a chemical or enzymatic process or a combination of the two. Conversion liberates the active drug from the masking “promoiety” or drug carrier in a way that the resulting molecule—the active metabolite—projects the full complement of the desired therapeutic effects. For more detailed review of prodrug strategy and examples see Rautio, J., Meanwell, N., Di, L. et al. Nat Rev Drug Discov 2018, 17, 559-587.

TABLE 1
Compound Table.

| | | Carbon | R |
|---|---|---|---|
| | Structure | Number of X | |
| I.1 | 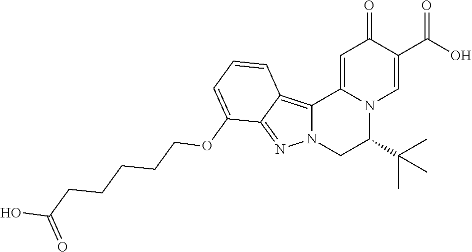 [SMILES: CC(C)(C)[C@@H]1Cn2nc3c(OCCCCCC(=O)O)cccc3c2-c2cc(=O)c(C(=O)O)cn21] | 5 | H |
| | (R)-6-(tert-butyl)-10-((5-carboxypentyl)oxy)-2-oxo-6,7-dihydro- | | |
| | 2H-pyrido[2′,1′:3,4]pyrazino[1,2-b]indazole-3-carboxylic acid | | |
| I.2 | 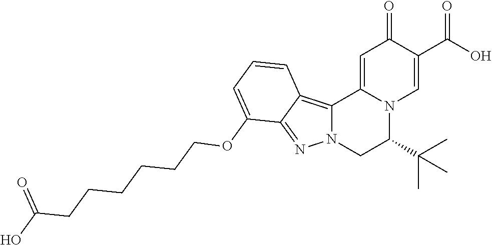 [SMILES: CC(C)(C)[C@@H]1Cn2nc3c(OCCCCCCC(=O)O)cccc3c2-c2cc(=O)c(C(=O)O)cn21] | 6 | H |
| | (R)-6-(tert-butyl)-10-((6-carboxyhexyl)oxy)-2-oxo-6,7-dihydro- | | |
| | 2H-pyrido[2′,1′:3,4]pyrazino[1,2-b]indazole-3-carboxylic acid | | |
| I.3 | 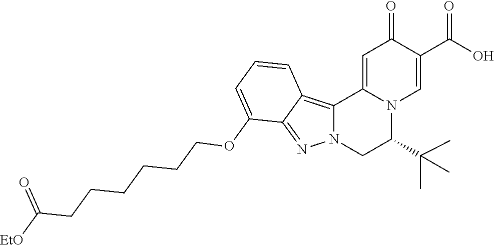 [SMILES: CCOC(=O)CCCCCCOc1cccc2c3n(nc12)C[C@@H](C(C)(C)C)n1cc(C(=O)O)c(=O)cc1-3] | 6 | C₂H₅ |
| | (R)-6-(tert-butyl)-10-((7-ethoxy-7-oxoheptyl)oxy)-2-oxo-6,7- | | |
| | dihydro-2H-pyrido[2′,1′:3,4]pyrazino[1,2-b]indazole-3-carboxylic | | |
| | acid | | |
| I.4 | 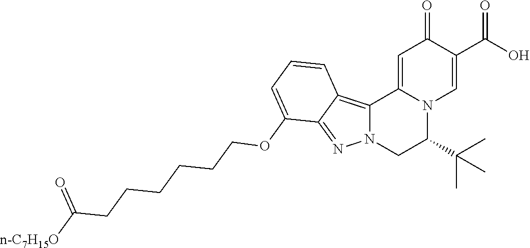 | 6 | n-C₇H₁₅ |
| | (R)-6-(tert-butyl)-10-((7-(heptyloxy)-7-oxoheptyl)oxy)-2-oxo-6,7- | | |
| | dihydro-2H-pyrido[2′,1′:3,4]pyrazino[1,2-b]indazole-3-carboxylic | | |
| | acid | | |
| I.5 | 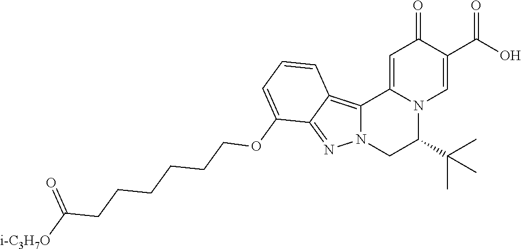 | 6 | i-C₃H₇ |
| | (R)-6-(tert-butyl)-10-((7-isopropoxy-7-oxoheptyl)oxy)-2-oxo-6,7- | | |
| | dihydro-2H-pyrido[2′,1′:3,4]pyrazino[1,2-b]indazole-3-carboxylic | | |
| | acid | | |
| I.6 | 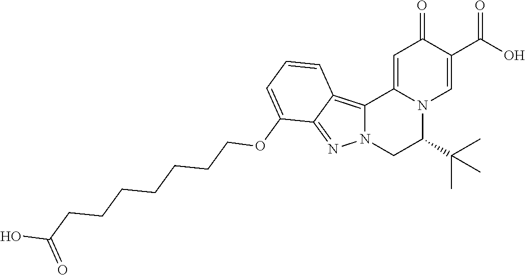 [SMILES: CC(C)(C)[C@@H]1Cn2nc3c(OCCCCCCCC(=O)O)cccc3c2-c2cc(=O)c(C(=O)O)cn21] | 7 | H |
| | (R)-6-(tert-butyl)-10-((7-carboxyheptyl)oxy)-2-oxo-6,7-dihydro- | | |
| | 2H-pyrido[2′,1′:3,4]pyrazino[1,2-b]indazole-3-carboxylic acid | | |
| I.7 | 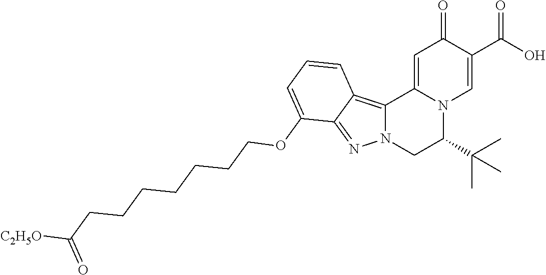 [SMILES: CCOC(=O)CCCCCCCOc1cccc2c3n(nc12)C[C@@H](C(C)(C)C)n1cc(C(=O)O)c(=O)cc1-3] | 7 | C₂H₅ |
| | (R)-6-(tert-butyl)-10-((8-ethoxy-8-oxooctyl)oxy)-2-oxo-6,7- | | |
| | dihydro-2H-pyrido[2′,1′:3,4]pyrazino[1,2-b]indazole-3-carboxylic | | |
| | acid | | |
| I.8 | 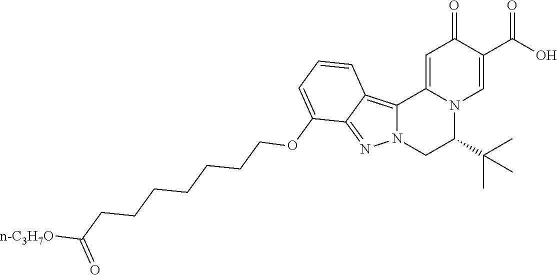 | 7 | n-C₃H₇ |
| | (R)-6-(tert-butyl)-2-oxo-10-((8-oxo-8-propoxyoctyl)oxy)-6,7- | | |
| | dihydro-2H-pyrido[2′,1′:3,4]pyrazino[1,2-b]indazole-3-carboxylic | | |
| | acid | | |
| I.9 | 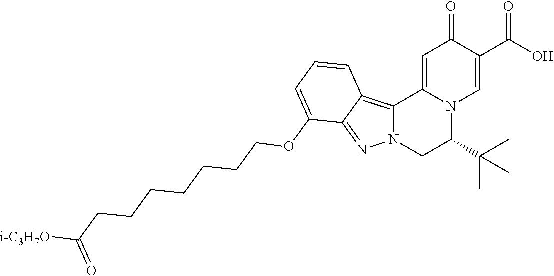 | 7 | i-C3H7 |
| | (R)-6-(tert-butyl)-10-((8-isopropoxy-8-oxooctyl)oxy)-2-oxo-6,7- | | |
| | dihydro-2H-pyrido[2′,1′:3,4]pyrazino[1,2-b]indazole-3-carboxylic | | |
| | acid | | |
| I.10 | 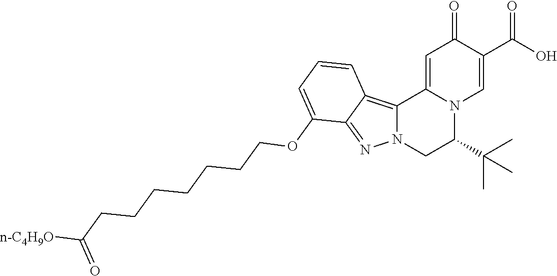 | 7 | n-C₄H₉ |
| | (R)-10-((8-butoxy-8-oxooctyl)oxy)-6-(tert-butyl)-2-oxo-6,7- | | |
| | dihydro-2H-pyrido[2′,1′:3,4]pyrazino[1,2-b]indazole-3-carboxylic | | |
| | acid | | |
| I.11 | 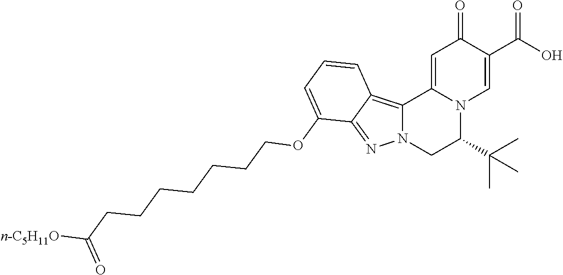 | 7 | n-C₅H₁₁ |
| | (R)-6-(tert-butyl)-2-oxo-10-((8-oxo-8-(pentyloxy)octyl)oxy)-6,7- | | |
| | dihydro-2H-pyrido[2',1':3,4]pyrazino[1,2-b]indazole-3-carboxylic | | |
| | acid | | |
| I.12 | 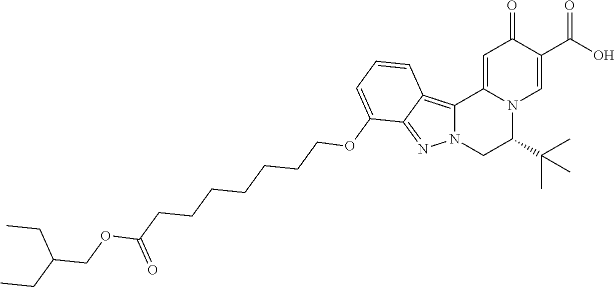 [SMILES: CCC(CC)COC(=O)CCCCCCCOc1cccc2c3n(nc12)C[C@@H](C(C)(C)C)n1cc(C(=O)O)c(=O)cc1-3] | 7 | —CH₂CH(C₂H₅)₂ |
| | (R)-6-(tert-butyl)-10-((8-(2-ethylbutoxy)-8-oxooctyl)oxy)-2-oxo- | | |
| | 6,7-dihydro-2H-pyrido[2′,1′:3,4]pyrazino[1,2-b]indazole-3- | | |
| | carboxylic acid | | |
| I.13 | 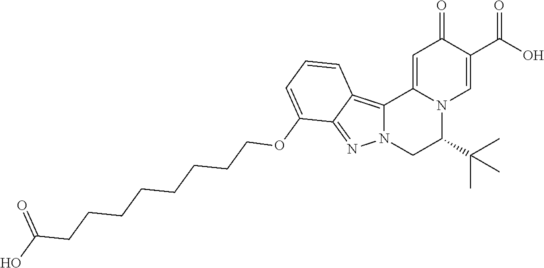 [SMILES: CC(C)(C)[C@@H]1Cn2nc3c(OCCCCCCCCC(=O)O)cccc3c2-c2cc(=O)c(C(=O)O)cn21] | 8 | H |
| | (R)-6-(tert-butyl)-10-((8-carboxyoctyl)oxy)-2-oxo-6,7-dihydro-2H- | | |
| | pyrido[2′,1′:3,4]pyrazino[1,2-b]indazole-3-carboxylic acid | | |
| I.14 | 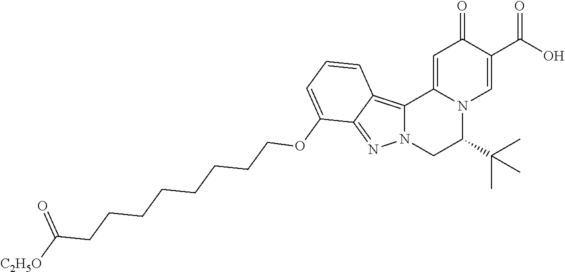 [SMILES: CCOC(=O)CCCCCCCCOc1cccc2c3n(nc12)C[C@@H](C(C)(C)C)n1cc(C(=O)O)c(=O)cc1-3] | 8 | C₂H₅ |
| | (R)-6-(tert-butyl)-10-((9-ethoxy-9-oxononyl)oxy)-2-oxo-6,7- | | |
| | dihydro-2H-pyrido[2′,1′:3,4]pyrazino[1,2-b]indazole-3-carboxylic | | |
| | acid | | |

Some of the compounds of Formula (I) in Table 1 are “prodrugs”, i.e. converted in vivo into some other compounds of Formula (I) (“parent drugs”) also listed in the Table 1.

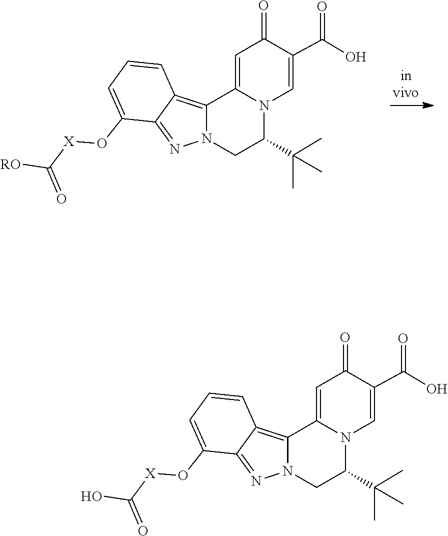

5 Each of the compounds of the Examples, including each of the “prodrugs” and “parent drugs” listed in Table 2, is a specific embodiment of the compounds of the invention.

TABLE 2
Selected examples of ″prodrugs″ and ″parent drugs″ among Compounds of Formula (I).

| | | |
|---|---|---|
| Example | ″Prodrug″ | ″Parent Drug″ |

| | | |
|---|---|---|
| 1 | 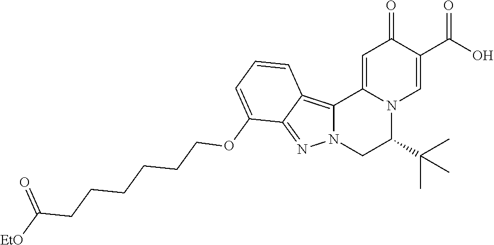 [SMILES: CCOC(=O)CCCCCCOc1cccc2c3n(nc12)C[C@@H](C(C)(C)C)n1cc(C(=O)O)c(=O)cc1-3] | 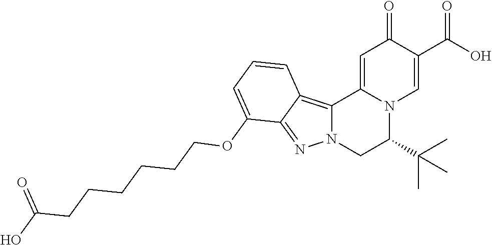 [SMILES: CC(C)(C)[C@@H]1Cn2nc3c(OCCCCCCC(=O)O)cccc3c2-c2cc(=O)c(C(=O)O)cn21] |
| | I.3 | I.2 |
| 2 | 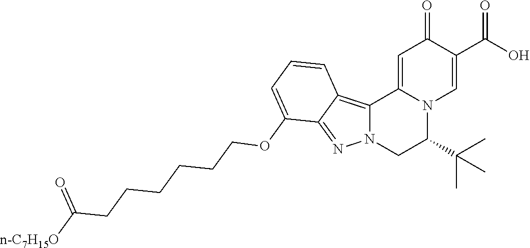 | 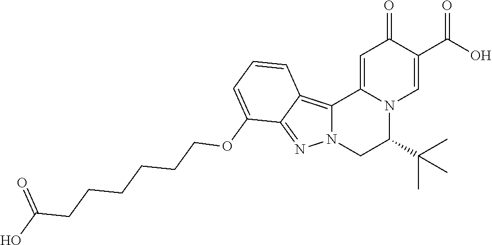 [SMILES: CC(C)(C)[C@@H]1Cn2nc3c(OCCCCCCC(=O)O)cccc3c2-c2cc(=O)c(C(=O)O)cn21] |
| | I.4 | I.2 |
| 3 | 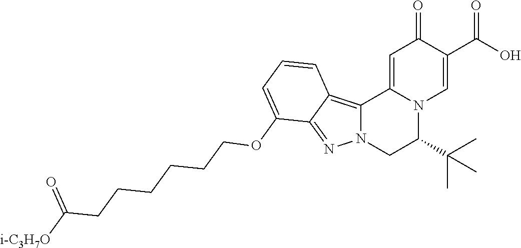 | 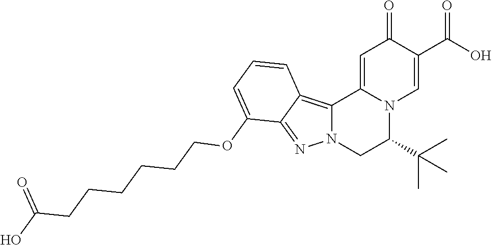 [SMILES: CC(C)(C)[C@@H]1Cn2nc3c(OCCCCCCC(=O)O)cccc3c2-c2cc(=O)c(C(=O)O)cn21] |
| | I.5 | I.2 |
| 4 | 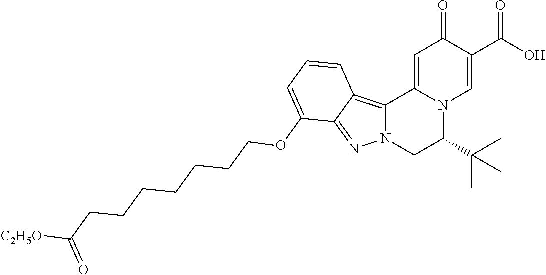 [SMILES: CCOC(=O)CCCCCCCOc1cccc2c3n(nc12)C[C@@H](C(C)(C)C)n1cc(C(=O)O)c(=O)cc1-3] | 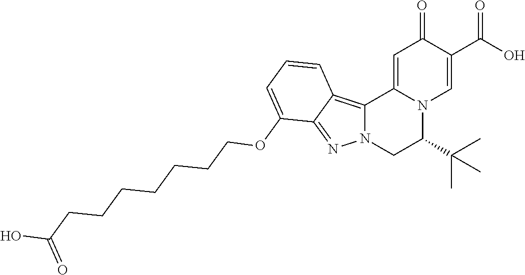 [SMILES: CC(C)(C)[C@@H]1Cn2nc3c(OCCCCCCCC(=O)O)cccc3c2-c2cc(=O)c(C(=O)O)cn21] |
| | I.7 | I.2 |
| 5 | 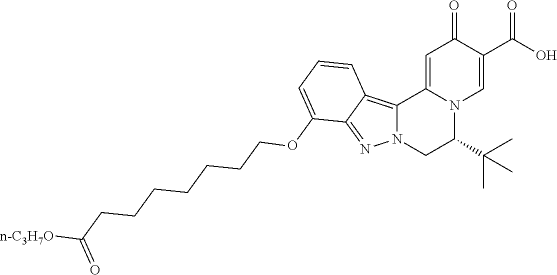 | 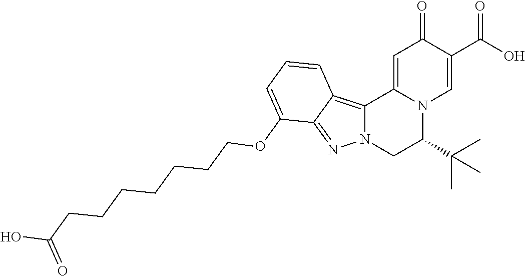 [SMILES: CC(C)(C)[C@@H]1Cn2nc3c(OCCCCCCCC(=O)O)cccc3c2-c2cc(=O)c(C(=O)O)cn21] |
| | I.8 | I.6 |
| 6 | 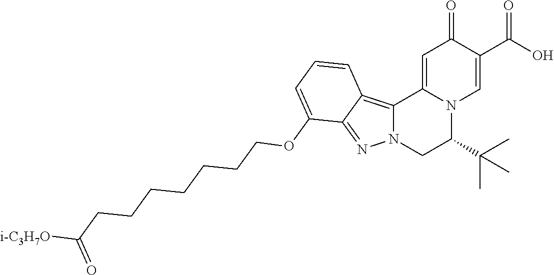 | 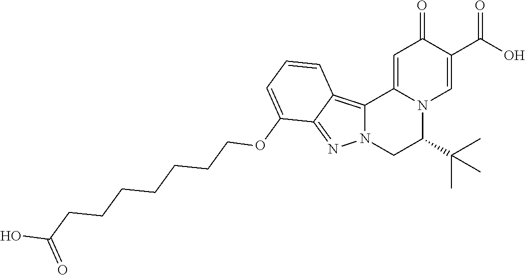 [SMILES: CC(C)(C)[C@@H]1Cn2nc3c(OCCCCCCCC(=O)O)cccc3c2-c2cc(=O)c(C(=O)O)cn21] |
| | I.9 | I.6 |
| 7 | 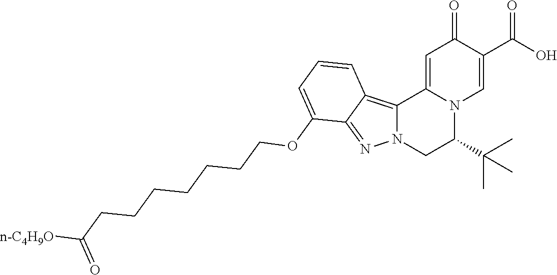 | 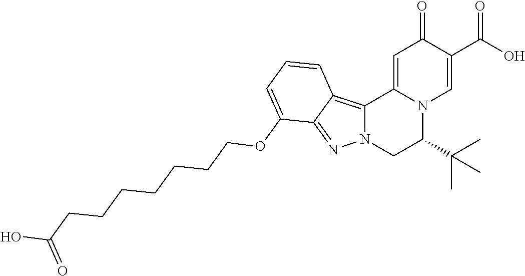 [SMILES: CC(C)(C)[C@@H]1Cn2nc3c(OCCCCCCCC(=O)O)cccc3c2-c2cc(=O)c(C(=O)O)cn21] |
| | I.10 | I.6 |
| 8 | 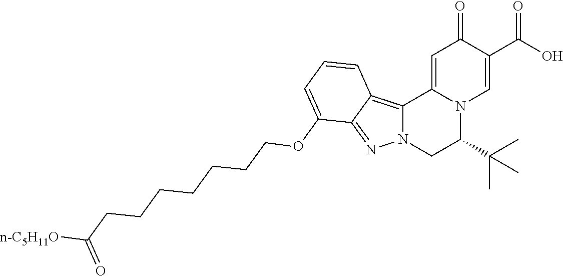 | 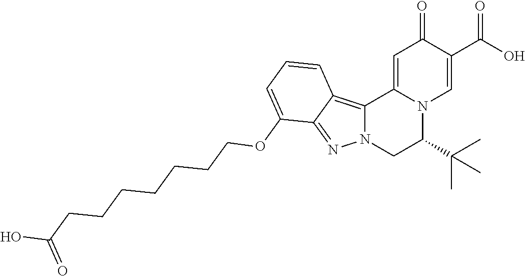 [SMILES: CC(C)(C)[C@@H]1Cn2nc3c(OCCCCCCCC(=O)O)cccc3c2-c2cc(=O)c(C(=O)O)cn21] |
| | I.11 | I.6 |
| 9 | 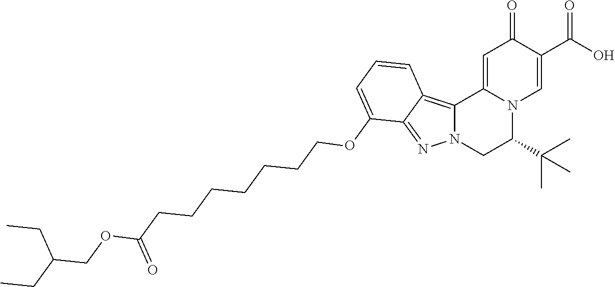 [SMILES: CCC(CC)COC(=O)CCCCCCCOc1cccc2c3n(nc12)C[C@@H](C(C)(C)C)n1cc(C(=O)O)c(=O)cc1-3] | 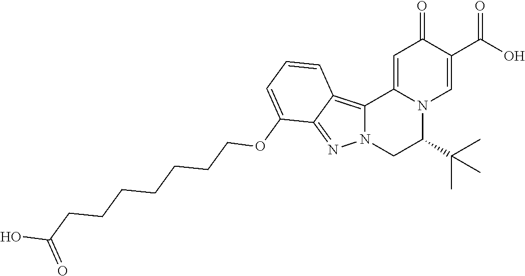 [SMILES: CC(C)(C)[C@@H]1Cn2nc3c(OCCCCCCCC(=O)O)cccc3c2-c2cc(=O)c(C(=O)O)cn21] |
| | I.12 | I.6 |
| 10 | 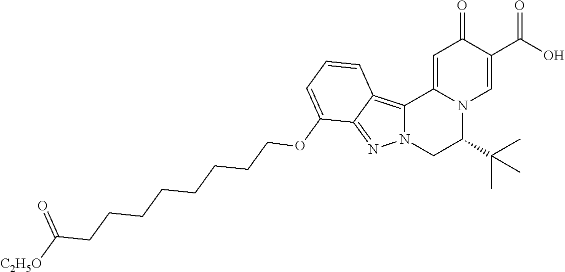 [SMILES: CCOC(=O)CCCCCCCCOc1cccc2c3n(nc12)C[C@@H](C(C)(C)C)n1cc(C(=O)O)c(=O)cc1-3] | 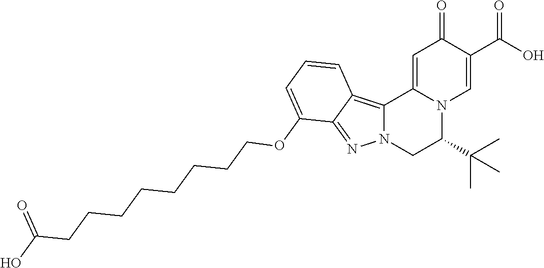 [SMILES: CC(C)(C)[C@@H]1Cn2nc3c(OCCCCCCCCC(=O)O)cccc3c2-c2cc(=O)c(C(=O)O)cn21] |
| | I.14 | I.13 |

For purposes of interpreting this specification, the following definitions will apply, and whenever appropriate, terms used in the singular will also include the plural.

Terms used in the specification have the following meanings unless the context clearly indicates otherwise:

As used herein, the term “subject” refers to an animal. In certain aspects, the animal is a mammal. A subject also refers to for example, primates (e.g., humans), cows, sheep, goats, horses, dogs, cats, rabbits, rats, mice, fish, birds and the like. In certain embodiments, the subject is a human. A “patient” as used herein refers to a human subject.

As used herein, the term “inhibition” or “inhibiting” refers to the reduction or suppression of a given condition, symptom, or disorder, or disease, or a significant decrease in the baseline activity of a biological activity or process.

As used herein, the term “treating” or “treatment” of any disease or disorder refers in one embodiment, to ameliorating the disease or disorder (i.e., slowing or arresting or reducing the development of the disease or at least one of the clinical symptoms thereof). In another embodiment “treating” or “treatment” refers to alleviating or ameliorating at least one physical parameter including those which may not be discernible by the patient. In yet another embodiment, “treating” or “treatment” refers to modulating the disease or disorder, either physically, (e.g., stabilization of a discernible symptom), physiologically, (e.g., stabilization of a physical parameter), or both. In yet another embodiment, “treating” or “treatment” refers to preventing or delaying the onset or development or progression of the disease or disorder.

As used herein, the term “a,” “an,” “the” and similar terms used in the context of the present invention (especially in the context of the claims) are to be construed to cover both the singular and plural unless otherwise indicated herein or clearly contradicted by the context.

All methods described herein can be performed in any suitable order unless otherwise indicated herein or otherwise clearly contradicted by context. The use of any and all examples, or exemplary language (e.g. “such as”) provided herein is intended merely to better illuminate the invention and does not pose a limitation on the scope of the invention otherwise claimed. “Optionally substituted” means the group referred to can be substituted at one or more positions by any one or any combination of the radicals listed thereafter. The number, placement and selection of substituents is understood to encompass only those substitutions that a skilled chemist would expect to be reasonably stable; thus ‘oxo’ would not be a substituent on an aryl or heteroaryl ring, for example, and a single carbon atom would not have three hydroxy or amino substituents. Unless otherwise specified, optional substituents are typically up to four groups selected from halo, oxo, CN, amino, hydroxy, —C₁₋₃ alkyl, —OR*, —NR*₂, —SR*, —SO₂R*, —COOR*, and —CONR* 2, where each R* is independently H or C₁₋₃ alkyl.

“Aryl” as used herein refers to a phenyl or naphthyl group unless otherwise specified.

Aryl groups unless otherwise specified may be optionally substituted with up to four groups selected from halo, CN, amino, hydroxy, C₁₋₃ alkyl, —OR*, —NR*₂, —SR*, —SO₂R*, —COOR*, and —CONR*₂, where each R* is independently H or C₁₋₃ alkyl.

“Halo” or “halogen”, as used herein, may be fluorine, chlorine, bromine or iodine.

“C₃₋₈ alkyl” or “C₃-C₈ alkyl”, as used herein, denotes straight chain or branched alkyl having 3-8 carbon atoms. If a different number of carbon atoms is specified, such as C₅ or C₅, then the definition is to be amended accordingly, such as “C₁₋₄ alkyl” will represent methyl, ethyl, propyl, isopropyl, butyl, isobutyl, sec-butyl and tert-butyl.

“C₅₋₁₀ alkylene” or “C₅-C₁₀ alkylene”, as used herein, denotes straight chain or branched alkyl having 5-10 carbon atoms and two open valences for connection to two other groups. If a different number of carbon atoms is specified, such as C4 or C3, then the definition is to be amended accordingly, such as “C₁₋₄ alkylene” will represent methylene (—CH₂—), ethylene (—CH₂CH₂—), straight chain or branched propylene (—CH₂CH₂CH₂— or —CH₂—CHMe—CH₂—), and the like.

“C₃₋₈ alkoxy”, as used herein, denotes straight chain or branched alkoxy (—O-Alkyl) having ₅₋₈ carbon atoms. If a different number of carbon atoms is specified, such as C4 or C3, then the definition is to be amended accordingly, such as “C₁₋₄ alkoxy” will represent methoxy, ethoxy, propoxy, isopropoxy, butoxy, isobutoxy, sec-butoxy and tert-butoxy.

“C₁₋₄ Haloalkyl” or “C₁-C₄ haloalkyl” as used herein, denotes straight chain or branched alkyl having 1-4 carbon atoms wherein at least one hydrogen has been replaced with a halogen. The number of halogen replacements can be from one up to the number of hydrogen atoms on the unsubstituted alkyl group. If a different number of carbon atoms is specified, such as C6 or C3, then the definition is to be amended accordingly. Thus “C₁₋₄ haloalkyl” will represent methyl, ethyl, propyl, isopropyl, butyl, isobutyl, sec-butyl and tert-butyl that have at least one hydrogen substituted with halogen, such as where the halogen is fluorine: CF₃CF₂—, (CF₃)₂CH—, CH₃—CF₂—, CF₃CF₂—, CF₃, CF₂H—, CF₃CF₂CH(CF₃)— or CF₃CF₂CF₂CF₂—.

“C₃₋₈ cycloalkyl” as used herein refers to a saturated monocyclic hydrocarbon ring of 3 to 8 carbon atoms. Examples of such groups include cyclopropyl, cyclobutyl, cyclopentyl and cyclohexyl. If a different number of carbon atoms is specified, such as C₃-C₆, then the definition is to be amended accordingly.

“4- to 8-Membered heterocyclyl”, “5- to 6-membered heterocyclyl”, “3- to 10-membered heterocyclyl”, “3- to 14-membered heterocyclyl”, “4- to 14-membered heterocyclyl” and “5- to 14-membered heterocyclyl”, refers, respectively, to 4- to 8-membered, 5- to 6-membered, 3- to 10-membered, 3- to 14-membered, 4- to 14-membered and 5- to 14-membered heterocyclic rings; unless otherwise specified, such rings contain 1 to 7, 1 to 5, or 1 to 3 heteroatoms selected from the group consisting of nitrogen, oxygen and sulfur as ring members, and the rings may be saturated, or partially saturated but not aromatic. The heterocyclic group can be attached to another group at a nitrogen or a carbon atom. The term “heterocyclyl” includes single ring groups, fused ring groups and bridged groups. Examples of such heterocyclyl include, but are not limited to pyrrolidine, piperidine, piperazine, pyrrolidinone, morpholine, tetrahydrofuran, tetrahydrothiophene, tetrahydrothiopyran, tetrahydropyran, 1,4- dioxane, 1,4-oxathiane, 8-aza-bicyclo[3.2.l]octane, 3,8-diazabicyclo[3.2.l]octane, 3-0xa-8-aza-bicyclo[3.2.l]octane, 8-Oxa-3-aza-bicyclo[3.2.l]octane, 2-Oxa-5-aza-bicyclo[2.2.l]heptane, 2,5-Diaza-bicyclo[2.2.l]heptane, azetidine, ethylenedioxo, oxetane or thiazole. In certain embodiments, if not otherwise specified, heterocyclic groups have 1-2 heteroatoms selected from N, O and S as ring members, and 4-7 ring atoms, and are optionally substituted with up to four groups selected from halo, oxo, CN, amino, hydroxy, C₁₋₃ alkyl, —OR*, —NR*₂, —SR*, —SO₂R*, —COOR*, and —CONR*₂, where each R* is independently H or C₁₋₃ alkyl. In particular, heterocyclic groups containing a sulfur atom are optionally substituted with one or two oxo groups on the sulfur.

“4-6 membered cyclic ether” as used herein refers to a 4 to 6 membered ring comprising one oxygen atom as a ring member. Examples include oxetane, tetrahydrofuran and tetrahydropyran.

“Heteroaryl” is a completely unsaturated (aromatic) ring. The term “heteroaryl” refers to a 5-14 membered monocyclic- or bicyclic- or tricyclic-aromatic ring system, having 1 to 8 heteroatoms selected from N, O or S. Typically, the heteroaryl is a 5-10 membered ring or ring system (e.g., 5-7 membered monocyclic group or an 8-10 membered bicyclic group), often a 5-6 membered ring containing up to four heteroatoms selected from N, O and S, though often a heteroaryl ring contains no more than one divalent O or S in the ring. Typical heteroaryl groups include furan, isothiazole, thiadiazole, oxadiazole, indazole, indole, quinoline, 2- or 3-thienyl, 2- or 3-furyl, 2- or 3-pyrrolyl, 2-, 4-, or 5-imidazolyl, 3-, 4-, or 5-pyrazolyl, 2-, 4-, or 5-thiazolyl, 3-, 4-, or 5-isothiazolyl, 2-, 4-, or 5-oxazolyl, 3-, 4-, or 5-isoxazolyl, 3- or 5-(1,2,4-triazolyl), 4- or 5-(1,2, 3-triazolyl), tetrazolyl, triazine, pyrimidine, 2-, 3-, or 4-pyridyl, 3- or 4-pyridazinyl, 3-, 4-, or 5-pyrazinyl, 2-pyrazinyl, and 2-, 4-, or 5-pyrimidinyl. Heteroaryl groups are optionally substituted with up to four groups selected from halo, CN, amino, hydroxy, C₁₋₃ alkyl, —OR*, —NR*₂, —SR*, —SO₂R*, —COOR*, and —CONR*₂, where each R* is independently H or C₁₋₃ alkyl. The term “hydroxy” or “hydroxyl” refers to the group —OH.

Furthermore, the compounds of the present invention, including their salts, can also be obtained in the form of their hydrates, or include other solvents used for their crystallization. The compounds of the present invention may inherently or by design form solvates with pharmaceutically acceptable solvents (including water); therefore, it is intended that the invention embrace both solvated and unsolvated forms. The term “solvate” refers to a molecular complex of a compound of the present invention (including pharmaceutically acceptable salts thereof) with one or more solvent molecules. Such solvent molecules are those commonly used in the pharmaceutical art, which are known to be innocuous to the recipient, e.g., water, ethanol, and the like. The term “hydrate” refers to the complex where the solvent molecule is water.

As used herein, the terms “salt” or “salts” refers to an acid addition or base addition salt of a compound of the present invention. “Salts” include in particular “pharmaceutically acceptable salts”. The term “pharmaceutically acceptable salts” refers to salts that retain the biological effectiveness and properties of the compounds of this invention and, which typically are not biologically or otherwise undesirable. In many cases, the compounds of the present invention are capable of forming acid and/or base salts by virtue of the presence of amino and/or carboxyl groups or groups similar thereto.

Pharmaceutically acceptable acid addition salts can be formed with inorganic acids and organic acids, e.g., acetate, aspartate, benzoate, besylate, bromide/hydrobromide, bicarbonate/carbonate, bisulfate/sulfate, camphorsulfonate, chloride/hydrochloride, chlortheophyllonate, citrate, ethandisulfonate, fumarate, gluceptate, gluconate, glucuronate, hippurate, hydroiodide/iodide, isethionate, lactate, lactobionate, laurylsulfate, malate, maleate, malonate, mandelate, mesylate, methylsulphate, naphthoate, napsylate, nicotinate, nitrate, octadecanoate, oleate, oxalate, palmitate, pamoate, phosphate/hydrogen phosphate/dihydrogen phosphate, polygalacturonate, propionate, stearate, succinate, sulfosalicylate, tartrate, tosylate and trifluoroacetate salts.

Inorganic acids from which salts can be derived include, for example, hydrochloric acid, hydrobromic acid, sulfuric acid, nitric acid, phosphoric acid, and the like.

Organic acids from which salts can be derived include, for example, acetic acid, propionic acid, glycolic acid, oxalic acid, maleic acid, malonic acid, succinic acid, fumaric acid, tartaric acid, citric acid, benzoic acid, mandelic acid, methanesulfonic acid, ethanesulfonic acid, toluenesulfonic acid, sulfosalicylic acid, and the like. Pharmaceutically acceptable base addition salts can be formed with inorganic and organic bases.

Inorganic bases from which salts can be derived include, for example, ammonium salts and metals from columns I to XII of the periodic table. In certain embodiments, the salts are derived from sodium, potassium, ammonium, calcium, magnesium, iron, silver, zinc, and copper; particularly suitable salts include ammonium, potassium, sodium, calcium and magnesium salts.

Organic bases from which salts can be derived include, for example, primary, secondary, and tertiary amines, substituted amines including naturally occurring substituted amines, cyclic amines, basic ion exchange resins, and the like. Certain organic amines include isopropylamine, benzathine, cholinate, diethanolamine, diethylamine, lysine, meglumine, piperazine and tromethamine.

The pharmaceutically acceptable salts of the present invention can be synthesized from a basic or acidic moiety, by conventional chemical methods. Generally, such salts can be prepared by reacting free acid forms of these compounds with a stoichiometric amount of the appropriate base (such as Na, Ca, Mg, or K hydroxide, carbonate, bicarbonate or the like), or by reacting free base forms of these compounds with a stoichiometric amount of the appropriate acid. Such reactions are typically carried out in water or in an organic solvent, or in a mixture of the two. Generally, use of non-aqueous media like ether, ethyl acetate, ethanol, isopropanol, or acetonitrile is desirable, where practicable. Lists of additional suitable salts can be found, e.g., in “Remington's Pharmaceutical Sciences”, 20th ed., Mack Publishing Company, Easton, Pa., (1985); and in “Handbook of Pharmaceutical Salts: Properties, Selection, and Use” by Stahl and Wermuth (Wiley-VCH, Weinheim, Germany, 2002).

Any formula given herein is intended to represent unlabeled forms as well as isotopically labeled forms of the compounds of the present invention having up to three atoms with non-natural isotope distributions, e.g., sites that are enriched in deuterium or ¹³C or ¹⁵N. Isotopically labeled compounds have structures depicted by the formulas given herein except that one or more atoms are replaced by an atom having a selected atomic mass or mass number other than the natural-abundance mass distribution. Examples of isotopes that can be usefully over-incorporated into compounds of the invention include isotopes of hydrogen, carbon, nitrogen, oxygen, phosphorous, fluorine, and chlorine, such as ²H, ³H, ¹¹C, ¹³C, ¹⁴C, ¹⁵N, ¹⁸F ³¹P, ³²P, ³⁵S, ³⁶Cl, ¹²⁵I respectively. The invention includes various isotopically labeled compounds of the present invention, for example those into which radioactive isotopes, such as ³H and ¹⁴C, or those in which non-radioactive isotopes, such as ²H and ¹³C are present at levels substantially above normal isotope distribution. Such isotopically labelled compounds are useful in metabolic studies (with ¹⁴C, for example), reaction kinetic studies (with, for example ²H or ³H), detection or imaging techniques, such as positron emission tomography (PET) or single-photon emission computed tomography (SPECT) including drug or substrate tissue distribution assays, or in radioactive treatment of patients. In particular, an ¹⁸F labeled compound of the present invention may be particularly desirable for PET or SPECT studies. Isotopically-labeled compounds of the present invention can generally be prepared by conventional techniques known to those skilled in the art or by processes analogous to those described in the accompanying Examples and Preparations using an appropriate isotopically-labeled reagent in place of the non-labeled reagent typically employed. Labeled samples may be useful with quite low isotope incorporation, such as where a radiolabel is used to detect trace amounts of the compound.

Further, site-specific substitution with heavier isotopes, particularly deuterium (i.e., ²H or D), may afford certain therapeutic advantages resulting from greater metabolic stability, for example increased in vivo half-life or reduced dosage requirements or an improvement in therapeutic index. It is understood that deuterium in this context is regarded as a substituent of a compound of the present invention, and typically a sample of a compound having deuterium as a substituent has at least 50% deuterium incorporation at the labeled position(s). The concentration of such a heavier isotope, specifically deuterium, may be defined by the isotopic enrichment factor. The term “isotopic enrichment factor” as used herein means the ratio between the isotopic abundance and the natural abundance of a specified isotope. If a substituent in a compound of this invention is denoted deuterium, such compound has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

Pharmaceutically acceptable solvates in accordance with the invention include those wherein the solvent of crystallization may be isotopically substituted, e.g. D₂O, d₆-acetone, d₆-DMSO.

Compounds of the present invention that contain groups capable of acting as donors and/or acceptors for hydrogen bonds may be capable of forming co-crystals with suitable co-crystal formers. These co-crystals may be prepared from compounds of the present invention by known co-crystal forming procedures. Such procedures include grinding, heating, co-subliming, co-melting, or contacting in solution compounds of the present invention with the co-crystal former under crystallization conditions and isolating co-crystals thereby formed. Suitable co-crystal formers include those described in WO 2004/078163. Hence the invention further provides co-crystals comprising a compound of the present invention.

### Methods of Use

All methods described herein can be performed in any suitable order unless otherwise indicated herein or otherwise clearly contradicted by context. The use of any and all examples, or exemplary language (e.g. “such as”) provided herein is intended merely to better illuminate the invention and does not pose a limitation on the scope of the invention otherwise claimed.

The compounds of the invention can be administered by known methods, including oral, parenteral, inhalation, and the like. In certain embodiments, the compound of the invention is administered orally, as a pill, lozenge, troche, capsule, solution, or suspension. In other embodiments, a compound of the invention is administered by injection or infusion. Infusion is typically performed intravenously, often over a period of time between about 15 minutes and 4 hours. In other embodiments, a compound of the invention is administered intranasally or by inhalation; inhalation methods are particularly useful for treatment of respiratory infections. Compounds of the present invention exhibit oral bioavailability, so oral administration is sometimes preferred.

In certain embodiments of the present invention, a compound of the present invention is used in combination with a second antiviral agent, such as those named herein.

By the term “combination”, is meant either a fixed combination in one dosage unit form, as separate dosage forms suitable for use together either simultaneously or sequentially, or as a kit of parts for the combined administration where a compound of the present invention and a combination partner may be administered independently at the same time or separately within time intervals that especially allow that the combination partners show a cooperative, e.g., synergistic, effect, or any combination thereof.

The second antiviral agent may be administered in combination with the compounds of the present inventions wherein the second antiviral agent is administered prior to, simultaneously, or after the compound or compounds of the present invention. When simultaneous administration of a compound of the invention with a second agent is desired and the route of administration is the same, then a compound of the invention may be formulated with a second agent into the same dosage form. An example of a dosage form containing a compound of the invention and a second agent is a tablet or a capsule.

In some embodiments, a combination of a compound of the invention and a second antiviral agent may provide synergistic activity. The compound of the invention and second antiviral agent may be administered together, separate but simultaneously, or sequentially.

An “effective amount” of a compound is that amount necessary or sufficient to treat or prevent a viral infection and/or a disease or condition described herein. In an example, an effective amount of a compound of Formula I is an amount sufficient to treat viral infection in a subject. In another example, an effective amount is an amount sufficient to treat HBV in a subject in need of such treatment. The effective amount can vary depending on such factors as the size and weight of the subject, the type of illness, or the particular compound of the invention. For example, the choice of the compound of the invention can affect what constitutes an “effective amount.” One of ordinary skill in the art would be able to study the factors contained herein and make the determination regarding the effective amount of the compounds of the invention without undue experimentation.

The regimen of administration can affect what constitutes an effective amount. The compound of the invention can be administered to the subject either prior to or after the onset of a viral infection. Further, several divided dosages, as well as staggered dosages, can be administered daily or sequentially, or the dose can be continuously infused, or can be a bolus injection. Further, the dosages of the compound(s) of the invention can be proportionally increased or decreased as indicated by the exigencies of the therapeutic or prophylactic situation.

Compounds of the invention may be used in the treatment of states, disorders or diseases as described herein, or for the manufacture of pharmaceutical compositions for use in the treatment of these diseases. The invention provides methods of use of compounds of the present invention in the treatment of these diseases or for preparation of pharmaceutical compositions having compounds of the present invention for the treatment of these diseases.

The language “pharmaceutical composition” includes preparations suitable for administration to mammals, e.g., humans. When the compounds of the present invention are administered as pharmaceuticals to mammals, e.g., humans, they can be given per se or as a pharmaceutical composition containing, for example, 0.1 to 99.5% (more preferably, 0.5 to 90%) of at least one compound of Formula (I) or any subgenus thereof as active ingredient in combination with a pharmaceutically acceptable carrier, or optionally two or more pharmaceutically acceptable carriers.

The phrase “pharmaceutically acceptable carrier” is art recognized and includes a pharmaceutically acceptable material, composition or vehicle, suitable for administering compounds of the present invention to mammals. The carriers include liquid or solid filler, diluent, excipient, solvent or encapsulating material, involved in carrying or transporting the subject agent from one organ, or portion of the body, to another organ, or portion of the body. Each carrier must be “acceptable” in the sense of being compatible with the other ingredients of the formulation and not injurious to the patient. Some examples of materials which can serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations. Typically, pharmaceutically acceptable carriers are sterilized and/or substantially pyrogen-free.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: water soluble antioxidants, such as ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propyl gallate, I-tocopherol, and the like; and metal chelating agents, such as citric acid, ethylenediamine tetraacetic acid (EDTA), sorbitol, tartaric acid, phosphoric acid, and the like.

Formulations of the present invention include those suitable for oral, nasal, inhalation, topical, transdermal, buccal, sublingual, rectal, vaginal and/or parenteral administration. The formulations may conveniently be presented in unit dosage form and may be prepared by any methods well known in the art of pharmacy. The amount of active ingredient that can be combined with a carrier material to produce a single dosage form will generally be that amount of the compound that produces a therapeutic effect. Generally, out of one hundred per cent, this amount will range from about 1 per cent to about ninety-nine percent of active ingredient, preferably from about 5 per cent to about 70 per cent, most preferably from about 10 percent to about 30 percent.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, cachets, pills, tablets, lozenges (using a flavored base, for example, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A compound of the present invention may also be administered as a bolus, electuary or paste.

In solid dosage forms of the invention for oral administration (capsules, tablets, pills, dragees, powders, granules and the like), the active ingredient is mixed with one or more pharmaceutically acceptable carriers, such as sodium citrate or dicalcium phosphate, and/or any of the following: fillers or extenders, such as starches, lactose, sucrose, glucose, mannitol, and/or silicic acid; binders, such as, for example, carboxymethylcellulose, alginates, gelatin, polyvinyl pyrrolidone, sucrose and/or acacia; humectants, such as glycerol; disintegrating agents, such as agar-agar, calcium carbonate, potato or tapioca starch, alginic acid, certain silicates, and sodium carbonate; solution retarding agents, such as paraffin; absorption accelerators, such as quaternary ammonium compounds; wetting agents, such as, for example, cetyl alcohol and glycerol monostearate; absorbents, such as kaolin and bentonite clay; lubricants, such a talc, calcium stearate, magnesium stearate, solid polyethylene glycols, sodium lauryl sulfate, and mixtures thereof; and coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. Examples of embedding compositions that can be used include polymeric substances and waxes. The active ingredient can also be in micro-encapsulated form, if appropriate, with one or more of the above-described excipients.

Liquid dosage forms for oral administration of the compounds of the invention include pharmaceutically acceptable emulsions, microemulsions, solutions, suspensions, syrups and elixirs. In addition to the active ingredient, the liquid dosage forms may contain inert diluent commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions of the invention for rectal or vaginal administration may be presented as a suppository, which may be prepared by mixing one or more compounds of the invention with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Formulations of the present invention which are suitable for vaginal administration also include pessaries, tampons, creams, gels, pastes, foams or spray formulations containing such carriers as are known in the art to be appropriate.

Dosage forms for the topical or transdermal administration of a compound of this invention include powders, sprays, ointments, pastes, creams, lotions, gels, solutions, patches and inhalants. The active compound may be mixed under sterile conditions with a pharmaceutically acceptable carrier, and with any preservatives, buffers, or propellants that may be required.

The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of this invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the compound in the proper medium. Absorption enhancers can also be used to increase the flux of the compound across the skin. The rate of such flux can be controlled by either providing a rate controlling membrane or dispersing the active compound in a polymer matrix or gel.

Pharmaceutical compositions of this invention suitable for parenteral administration may comprise one or more compounds of the invention in combination with one or more pharmaceutically acceptable carriers such as sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions of the invention include water, ethanol, glycol ethers, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

The preparations of the present invention may be given orally, parenterally, topically, or rectally. They are of course given by forms suitable for each administration route. For example, they are administered in tablets or capsule form, by injection, inhalation, eye lotion, ointment, suppository, etc., administration by injection, infusion or inhalation; topical by lotion or ointment; and rectal by suppositories.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion. Intravenous infusion is sometimes a preferred method of delivery for compounds of the invention. Infusion may be used to deliver a single daily dose or multiple doses. In some embodiments, a compound of the invention is administered by infusion over an interval between 15 minutes and 4 hours, typically between 0.5 and 3 hours. Such infusion may be used once per day, twice per day or up to three times per day.

The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

These compounds may be administered to humans and other animals for therapy by any suitable route of administration, including orally, nasally, as by, for example, a spray, rectally, intravaginally, parenterally, intracisternally and topically, as by powders, ointments or drops, including buccally and sublingually.

Regardless of the route of administration selected, the compounds of the present invention, which may be used in a suitable hydrated form, and/or the pharmaceutical compositions of the present invention, are formulated into pharmaceutically acceptable dosage forms by conventional methods known to those of skill in the art.

Actual dosage levels of the active ingredients in the pharmaceutical compositions of this invention may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular compound of the present invention employed, or the ester, salt or amide thereof, the route of administration, the time of administration, the rate of excretion of the particular compound being employed, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In general, a suitable daily dose of a compound of the invention will be that amount of the compound that is the lowest dose effective to produce a therapeutic effect. Such an effective dose will generally depend upon the factors described above. Generally, intravenous and subcutaneous doses of the compounds of this invention for a patient, when used for the indicated effects, will range from about 0.0001 to about 100 mg per kilogram of body weight per day, more preferably from about 0.01 to about 50 mg per kg per day, and still more preferably from about 0.1 to about 20 mg per kg per day. An effective amount is that amount which prevents or treats a viral infection, such as HBV.

Treatment with a compound or composition described herein may be repeated daily for a period sufficient to reduce clear HBsAg either alone or in combination with other therapeutics.

The daily dose of the compound may be administered once or twice or three times daily, to be determined.

The compound(s) of the present invention may be administered alone or in combination (either sequentially or simultaneously) with other therapeutics. Thus, methods of using the compounds of the invention include administering the compound as a pharmaceutical composition, wherein at least one compound of the invention is admixed with a pharmaceutically acceptable carrier prior to administration.

### Use of Compounds of the Invention in Combination

The compounds and compositions described herein can be used or administered in combination with one or more therapeutic agents including but not limited to immunomodulators [(i.e., checkpoint inhibitors (small molecule inhibitors or blocking antibodies)], native or modified cytokines, TLR agonists, vaccines, etc.); and/or HBsAg targeting agents (i.e., siRNA and antisense molecules targeting HBV transcript(s), HBV capsid inhibitors, cccDNA inhibitors, HBx inhibitors and antibodies targeting HBV or HDV proteins, etc.) ; and/or HBV replication inhibitors (nucleos(t)ide analogue polymerase inhibitors, non-nucleos(t)ide analogue polymerase inhibitors, etc.).

In general, it is expected that each of the therapeutic agents utilized in combination be utilized at levels that do not exceed the levels at which they are utilized individually. In some embodiments, the doses utilized in combination may be lower than those utilized individually.

Treatment with a compound or composition described herein may be repeated daily for a period sufficient to reduce clear HBsAg either alone or in combination with other therapeutics.

The daily dose of the compound may be administered once or twice daily or three times daily, to be determined.

The compound(s) of the present invention may be administered alone or in combination (either sequentially or simultaneously) with other therapeutics. Thus, methods of using the compounds of the invention include administering the compound as a pharmaceutical composition, wherein at least one compound of the invention is admixed with a pharmaceutically acceptable carrier prior to administration.

The compounds as described herein may be synthesized by the general synthetic routes below, specific examples of which are described in more detail in the Examples.

### General Synthetic Procedures

All starting materials, building blocks, reagents, acids, bases, dehydrating agents, solvents, and catalysts utilized to synthesize the compounds of the invention are either commercially available or can be produced by organic synthesis methods known to one of ordinary skill in the art (Houben-Weyl 4th Ed. 1952, Methods of Organic Synthesis, Thieme, Volume 21). General methods for synthesis of compounds of the invention are illustrated by the Examples below, the general method in Scheme 1, and by methods disclosed in published PCT applications WO 2018/198079 and U.S. Pat. No. 10,301,312 B2 Date of Patent: May 28, 2019 whose contents are incorporated entirely by this reference.

### Preparation of Common Intermediate 3

### Synthetic Route:

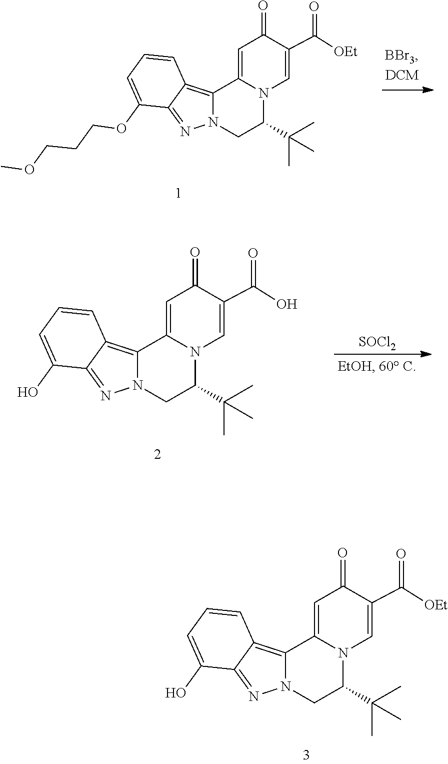 [SMILES: CCOC(=O)c1cn2c(cc1=O)-c1c3cccc(OCCCOC)c3nn1C[C@H]2C(C)(C)C ; CC(C)(C)[C@@H]1Cn2nc3c(O)cccc3c2-c2cc(=O)c(C(=O)O)cn21 ; "SOCl2" ; CCOC(=O)c1cn2c(cc1=O)-c1c3cccc(O)c3nn1C[C@H]2C(C)(C)C]

### Preparation of (R)-6-(tert-butyl)-10-hydroxy-2-oxo-6,7-dihydro-2H-pyrido[2′,1′:3,4]pyrazino[1,2-b]indazole-3-carboxylic Acid (2)

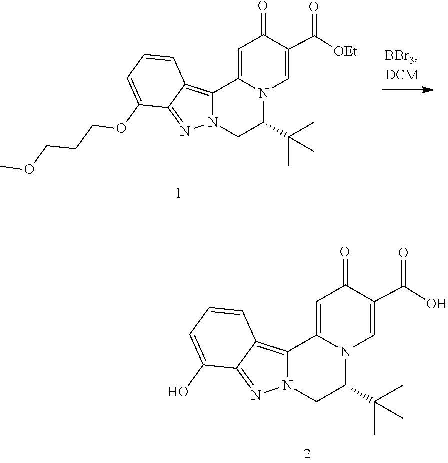 [SMILES: CCOC(=O)c1cn2c(cc1=O)-c1c3cccc(OCCCOC)c3nn1C[C@H]2C(C)(C)C ; CC(C)(C)[C@@H]1Cn2nc3c(O)cccc3c2-c2cc(=O)c(C(=O)O)cn21]

The reaction was split into two equal batches. In each of them, a mixture of ethyl (R)-6-(tert-butyl)-10-(3-methoxypropoxy)-2-oxo-6,7-dihydro-2H-pyrido[2′,1′:3,4]pyrazino[1,2-b]indazole-3-carboxylate (1) (45 g, 99.22 mmol) in DCM (1 L) was dropwise added BBr 3 (99.43 g, 397 mmol, 38.24 mL, 4.0 eq) at 0 ° C. under N₂. The mixture was warmed to 40° C. and stirred for 12 h. A solid precipitate appeared. TLC (Petroleum ether:Ethyl acetate=0:1, R_{f}=0.1) showed the reaction was completed. The 2 reactions were combined for work-up. The mixture was diluted with dichloromethane (DCM, 1 L) and stirred for 5 min. The solid was collected by filtration and washed with DCM (3×500 mL). The filter cake was dried over vacuum. To the residue was added THF (450 mL), H₂O (450 mL) and LiOH·H₂O (16.65 g, 416.2 mmol, 4.2 eq). The solution was stirred at 25° C. for 2 hrs. The residue was adjusted to pH=4 with HCl (2M). The residue was filtered to yield (R)-6-(tert-butyl)-10-hydroxy-2-oxo-6,7-dihydro-2H-pyrido[2′,1′:3,4]pyrazino[1,2-b]indazole-3-carboxylic acid (2) (60 g, yield: 86%) as a yellow solid.

¹H NMR (400 MHz, DMSO-d₆) δ ppm 10.36; (s, 1 H), 8.94; (s, 1 H), 7.49; (d, J=8.4 Hz, 1 H), 7.23; (s, 1 H), 7.25-7.12; (m, 1 H), 7.27-7.11; (m, 1 H), 7.16; (t, J=8.0 Hz, 1 H), 6.70; (d, J=7.2 Hz, 1 H), 5.14-5.06; (m, 2 H), 4.96; (br d, J=3.6 Hz, 1 H), 0.71; (s, 9 H).

### Preparation of Ethyl (R)-6-(tert-butyl)-10-hydroxy-2-oxo-6,7-dihydro-2H-pyrido[2′,1′3,4]pyrazino[1,2-b]indazole-3-carboxylate (3).

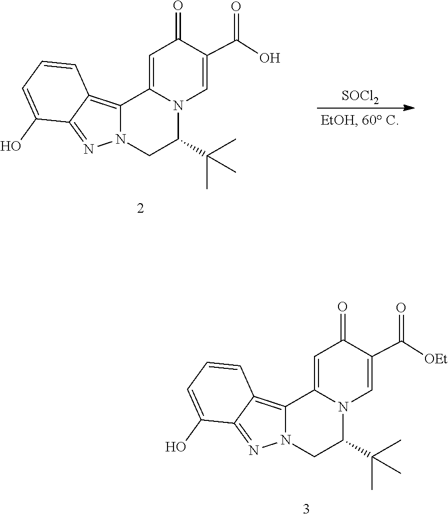 [SMILES: CC(C)(C)[C@@H]1Cn2nc3c(O)cccc3c2-c2cc(=O)c(C(=O)O)cn21 ; "SOCl2" ; CCOC(=O)c1cn2c(cc1=O)-c1c3cccc(O)c3nn1C[C@H]2C(C)(C)C]

To a solution of ((R)-6-(tert-butyl)-10-hydroxy-2-oxo-6,7-dihydro-2H-pyrido[2′,1′:3,4]pyrazino[1,2-b]indazole-3-carboxylic acid (2) (60 g, 169.79 mmol) in EtOH (600 mL) was added SOCl₂ (101.00 g, 848.97 mmol, 61.6 mL, 5.0 eq) at 0° C. The mixture was stirred at 60° C. for 12 hrs. LCMS showed the reaction was complete. The mixture was concentrated under reduced pressure. The residue was purified by column chromatography on silica gel (Dichloromethane:EtOH=1:0 to 5:1) to give ethyl (R)-6-(tert-butyl)-10-hydroxy-2-oxo-6,7-dihydro-2H-pyrido[2′,1′:3,4]pyrazino[1,2-b]indazole-3-carboxylate (3) (55 g, yield: 84.92%) as a yellow solid.

LCMS: RT=0.194 min, MS calc.: 381.2, MS found: [M+H]⁺=382.3

¹H NMR (400 MHz, DMSO-d₆) δ ppm 8.77; (s, 1 H), 7.39; (d, J=8.4 Hz, 1 H), 7.28; (s, 1 H), 7.16; (t, J=8.0 Hz, 1 H), 6.70; (d, J=7.2 Hz, 1 H), 5.18-4.99; (m, 2 H), 4.87; d, J=2.8 Hz, 1 H), 4.29; (q, J=7.2 Hz, 2 H), 1.30; (t, J=7.2 Hz, 3 H), 0.72; (s, 9 H).

Compound 3M was prepared using this method substituting methanol for ethanol.

### Compound I.1

### Synthetic Route:

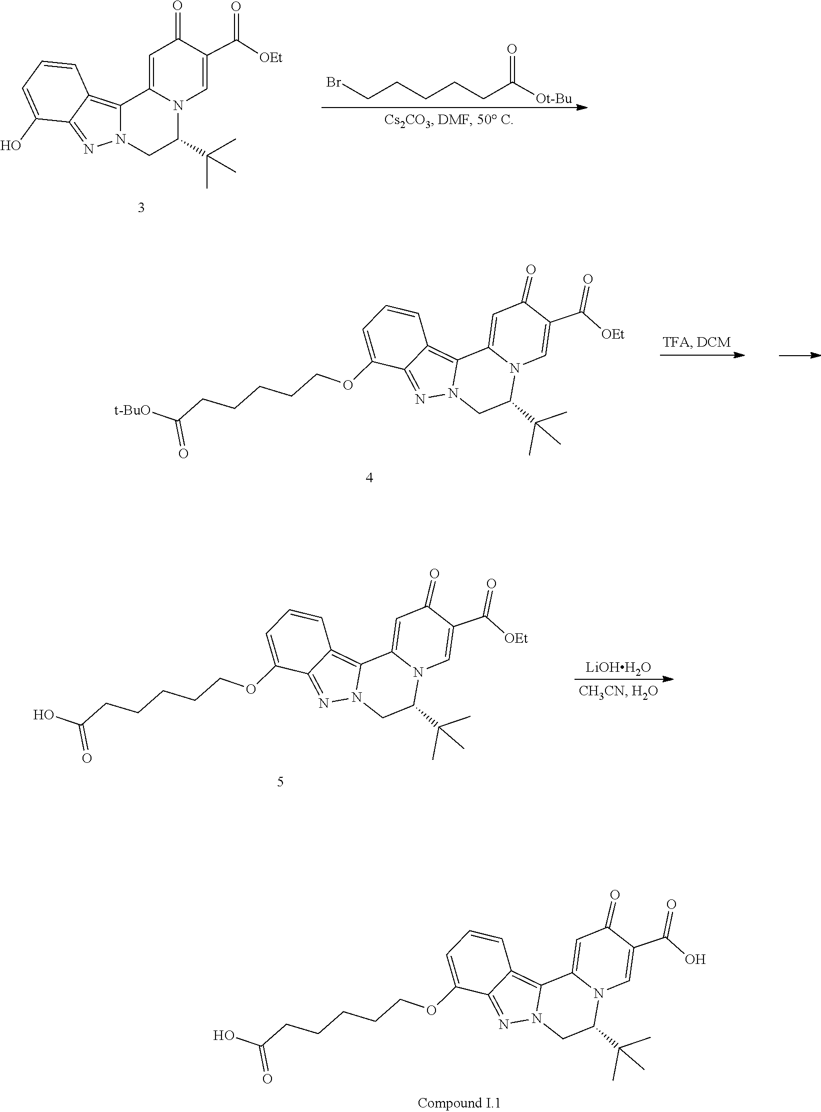 [SMILES: CCOC(=O)c1cn2c(cc1=O)-c1c3cccc(O)c3nn1C[C@H]2C(C)(C)C ; CC(C)(C)OC(=O)CCCCCBr ; CCOC(=O)c1cn2c(cc1=O)-c1c3cccc(OCCCCCC(=O)OC(C)(C)C)c3nn1C[C@H]2C(C)(C)C ; CCOC(=O)c1cn2c(cc1=O)-c1c3cccc(OCCCCCC(=O)O)c3nn1C[C@H]2C(C)(C)C ; "LiOH·H2O" ; CC(C)(C)[C@@H]1Cn2nc3c(OCCCCCC(=O)O)cccc3c2-c2cc(=O)c(C(=O)O)cn21]

### Procedure for Preparation of Ethyl (R)-10-((6-(tert-butoxy)-6-oxohexyl)oxy)-6-(tert-butyl)-2-oxo-6,7-dihydro-2H-pyrido[2′,1′:3,4]pyrazino[1,2-b]indazole-3-carboxylate (4)

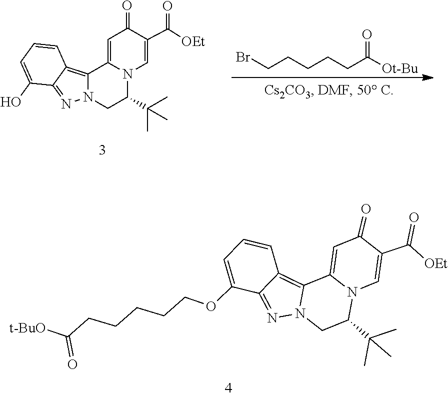 [SMILES: CCOC(=O)c1cn2c(cc1=O)-c1c3cccc(O)c3nn1C[C@H]2C(C)(C)C ; CC(C)(C)OC(=O)CCCCCBr ; CCOC(=O)c1cn2c(cc1=O)-c1c3cccc(OCCCCCC(=O)OC(C)(C)C)c3nn1C[C@H]2C(C)(C)C]

To a mixture of ethyl (R)-6-(tert-butyl)-10-hydroxy-2-oxo-6,7-dihydro-2H-pyrido[2′,1′:3,4]pyrazino[1,2-b]indazole-3-carboxylate (3) (1.5 g, 3.93 mmol, 1 eq.) and tert-butyl 6-bromohexanoate (2.47 g, 9.83 mmol, 2.5 eq.) in DMF (15 mL) was added Cs₂CO₃ (4.48 g, 13.76 mmol, 3.5 eq.) in one portion at 20° C. The mixture was stirred at 50° C. for 4 hours. LCMS showed the reaction was complete. The mixture was poured into water (20 mL) and extracted with ethyl acetate (10 mL×3). The combined organic phases were washed with brine (10 mL×3), dried with Na₂SO₄, filtered and concentrated in vacuum. The residue was purified by prep-HPLC (column: Waters Xbridge BEH C18 100*30 mm*10 um; mobile phase: [water (NH₄HCO₃)-ACN]; B %: 40%-70%, 8 min) to give ethyl (R)-10-((6-(tert-butoxy)-6-oxohexyl)oxy)-6-(tert-butyl)-2-oxo-6,7-dihydro-2H-pyrido[2′,1′:3,4]pyrazino[1,2-b]indazole-3-carboxylate (4) (1.4 g, 2.54 mmol, 64.5% yield) as a yellow solid.

¹H NMR (400 MHz, DMSO-d₆) δ ppm 8.53; (s, 1 H), 7.50; (d, J =8.4 Hz, 1 H), 7.18; (t, J=8.0 Hz, 1 H), 6.83-6.75; (m, 2 H), 5.13-4.98; (m, 2 H), 4.70; (d, J=4.0 Hz, 1 H), 4.24; (q, J=6.8 Hz, 2 H), 4.18-4.08; (m, 2 H), 2.23; (t, J=7.2 Hz, 2 H), 1.81-1.79; (m, 2 H), 1.63-1.53; (m, 2 H), 1.52-1.41; (m, 2 H), 1.38; (s, 9 H), 1.28; (t, J=6.8 Hz, 3 H), 0.69; (s, 9 H).

### Procedure for Preparation of (R)-64(6-(tert-butyl)-3-(ethoxycarbonyl)-2-oxo-6,7-dihydro-2H-pyrido[2′,1′:3,4]pyrazino[1,2-b]indazol-10-yl)oxy)hexanoic Acid (5)

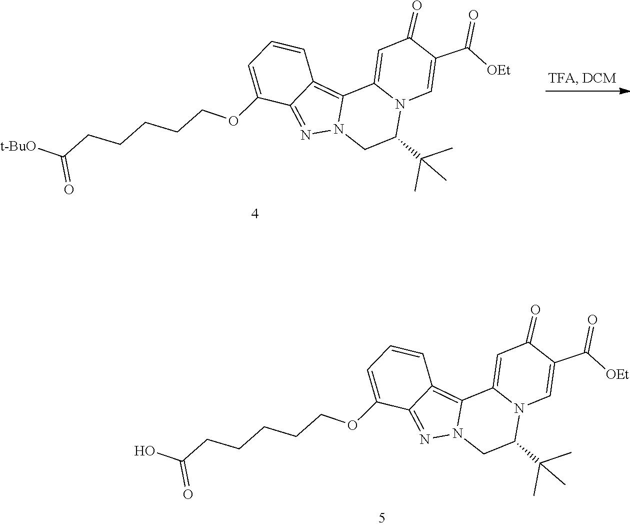 [SMILES: CCOC(=O)c1cn2c(cc1=O)-c1c3cccc(OCCCCCC(=O)OC(C)(C)C)c3nn1C[C@H]2C(C)(C)C ; CCOC(=O)c1cn2c(cc1=O)-c1c3cccc(OCCCCCC(=O)O)c3nn1C[C@H]2C(C)(C)C]

To a mixture of ethyl (R)-10-((6-(tert-butoxy)-6-oxohexyl)oxy)-6-(tert-butyl)-2-oxo-6,7-dihydro-2H-pyrido[2′,1′:3,4]pyrazino[1,2-b]indazole-3- carboxylate (4) (1.2 g, 2.18 mmol, 1 eq.) in DCM (10 mL) was added trifluoroacetic acid (TFA, 5 mL) in one portion at 20° C. The mixture was stirred at 20° C. for 2 hours. LCMS showed the reaction was completed. The mixture was concentrated and adjusted pH=7 by saturated NaHCO₃. The aqueous phase was extracted with DCM (10 mL×3). The combined organic phases were washed with brine (5 mL×3), dried with Na₂SO₄, filtered and concentrated in vacuum. The residue was purified by prep-HPLC (column: Waters Xbridge BEH C18 250*50 mm*10 um; mobile phase: [water (NH₄HCO₃)-ACN]; B %: 10%-40%, 10 min) to give (R)-6-((6-(tert-butyl)-3-(ethoxycarbonyl)-2-oxo-6,7-dihydro-2H-pyrido[2′,1′:3,4]pyrazino[1,2-b]indazol-10-yl)oxy)hexanoic acid (5) (0.61 g, 1.22 mmol, 56.02% yield) as a yellow solid.

¹H NMR (400 MHz, DMSO-d₆) δ ppm 8.53; (s, 1 H), 7.50; (d, J=8.4 Hz, 1 H), 7.18; (t, J=8.0 Hz, 1 H), 6.85-6.73; (m, 2 H), 5.16-4.98; (m, 2 H), 4.70; (d, J=4.4 Hz, 1 H), 4.24; (q, J=6.8 Hz, 2 H), 4.19-4.08; (m, 2 H), 2.24; (t, J=7.2 Hz, 2 H), 1.82-1.79; (m, 2 H), 1.65-1.54; (m, 2 H), 1.53-1.41; (m, 2 H), 1.28; (t, J=6.8Hz, 3 H), 0.70; (s, 9 H).

### Procedure for Preparation of (R)-6-(tert-butyl)-10-((5-carboxypentyl)oxy)-2-oxo-6,7-dihydro-2H-pyrido[2′,1′:3,4]pyrazino[1,2-b]indazole-3-carboxylic Acid (Compound I.1)

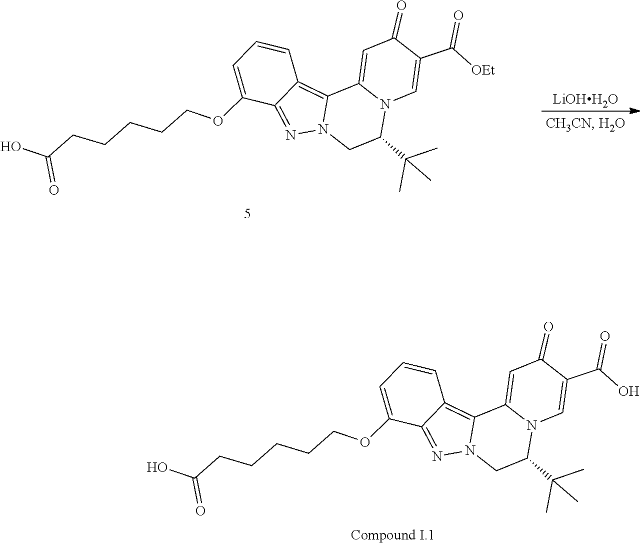 [SMILES: CCOC(=O)c1cn2c(cc1=O)-c1c3cccc(OCCCCCC(=O)O)c3nn1C[C@H]2C(C)(C)C ; "LiOH·H2O" ; CC(C)(C)[C@@H]1Cn2nc3c(OCCCCCC(=O)O)cccc3c2-c2cc(=O)c(C(=O)O)cn21]

To a mixture (R)-6-((6-(tert-butyl)-3-(ethoxycarbonyl)-2-oxo-6,7-dihydro-2H-pyrido[2′,1′:3,4]pyrazino[1,2-b]indazol-10-yl)oxy)hexanoic acid (0.15 g, 303 umol, 1 eq.) in CH₃CN (1.5 mL) and H₂O (0.3 mL) was added LiOH·H₂O (57 mg, 1.36 mmol, 4.5 eq.). The mixture was stirred at 35° C. for 1 hour. LCMS showed the reaction was complete. The mixture was adjusted pH=6-7 by 1N HCl, and concentrated in vacuum. The residue was purified by prep-HPLC (column: Waters Xbridge C18 150*50 mm*10 um; mobile phase: [water (NH₄HCO₃)-ACN]; B %: 1%-30%, 8 min) to give (R)-6-(tert-butyl)-10-((5-carboxypentyl)oxy)-2-oxo-6,7-dihydro-2H- pyrido[2′,1′:3,4]pyrazino[1,2-b]indazole-3-carboxylic acid (Compound I.1) (81 mg, 163 umol, 54% yield) as a yellow solid.

¹H NMR (400 MHz, DMSO-d₆) δ ppm 8.95; (s, 1 H), 7.63; (d, J=8.4 Hz, 1 H), 7.33-7.18; (m, 2 H), 6.83; (d, J=7.6 Hz, 1 H), 5.27-5.06; (m, 2 H), 4.96; (d, J=4.4 Hz, 1 H), 4.22- 4.09; (m, 2 H), 2.24; (t, J=7.2 Hz, 2 H), 1.82-1.79; (m, 2 H), 1.64-1.55; (m, 2 H), 1.54-1.42; (m, 2 H), 0.71; (s, 9 H).

### Compound I.2

### Synthetic Route:

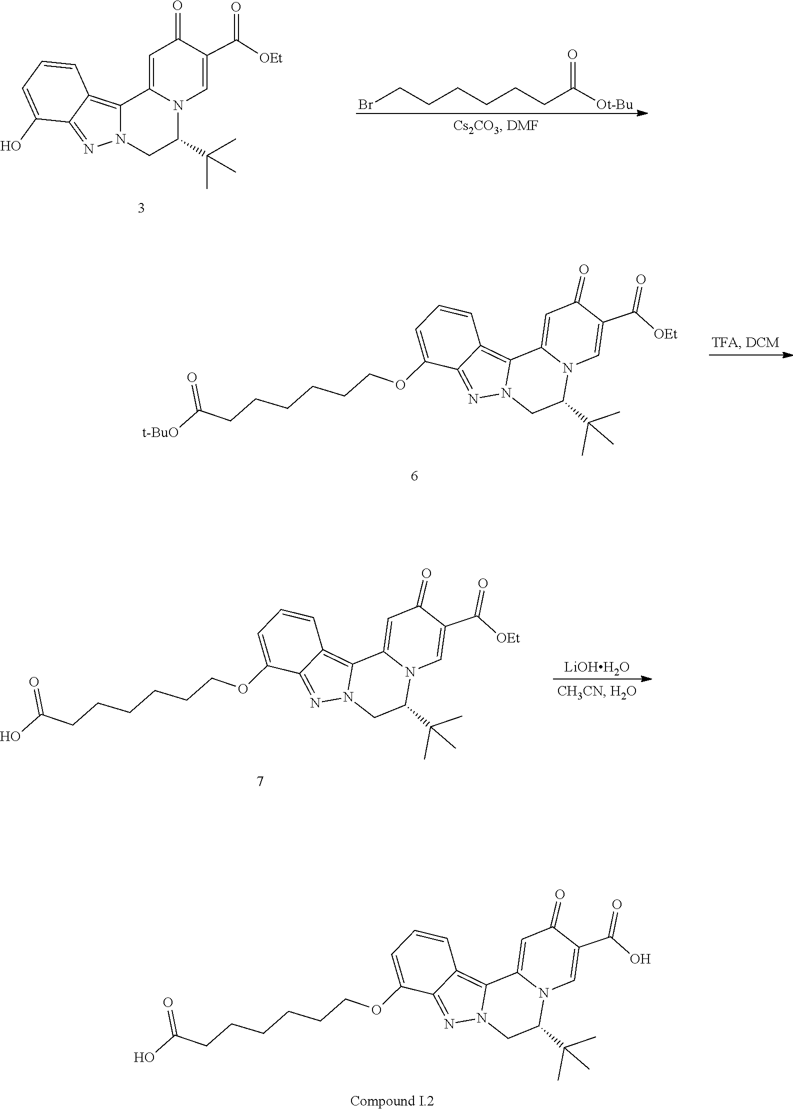 [SMILES: CCOC(=O)c1cn2c(cc1=O)-c1c3cccc(O)c3nn1C[C@H]2C(C)(C)C ; CC(C)(C)OC(=O)CCCCCCBr ; CCOC(=O)c1cn2c(cc1=O)-c1c3cccc(OCCCCCCC(=O)OC(C)(C)C)c3nn1C[C@H]2C(C)(C)C ; CCOC(=O)c1cn2c(cc1=O)-c1c3cccc(OCCCCCCC(=O)O)c3nn1C[C@H]2C(C)(C)C ; "LiOH·H2O" ; CC(C)(C)[C@@H]1Cn2nc3c(OCCCCCCC(=O)O)cccc3c2-c2cc(=O)c(C(=O)O)cn21]

### Procedure for Preparation of Ethyl (R)-10-((7-(tert-butoxy)-7-oxoheptyl)oxy)-6-(tert-butyl)-2-oxo-6,7-dihydro-2H-pyrido[2′,1′:3,4]pyrazino[1,2-b]indazole-3-carboxylate (6)

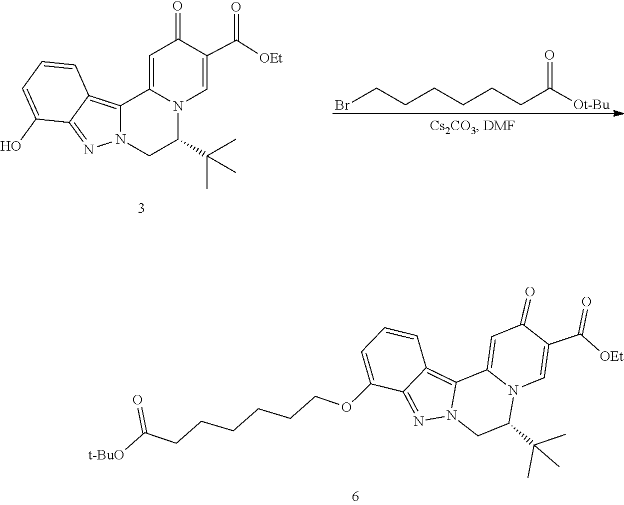 [SMILES: CCOC(=O)c1cn2c(cc1=O)-c1c3cccc(O)c3nn1C[C@H]2C(C)(C)C ; CC(C)(C)OC(=O)CCCCCCBr ; CCOC(=O)c1cn2c(cc1=O)-c1c3cccc(OCCCCCCC(=O)OC(C)(C)C)c3nn1C[C@H]2C(C)(C)C]

To a solution of ethyl (R)-6-(tert-butyl)-10-hydroxy-2-oxo-6,7-dihydro-2H-pyrido[2′,1′:3,4]pyrazino[1,2-b]indazole-3-carboxylate (3) (1.5 g, 3.93 mmol, 1 eq.) and tert-butyl 7-bromoheptanoate (1.56 g, 5.9 mmol, 1.5 eq.) in DMF (20 mL) was added Cs₂CO₃ (4.48 g, 13.76 mmol, 3.5 eq.) at 25° C. The mixture was stirred at 50° C. for 12 hrs. LCMS showed the reaction was complete. The reaction mixture was concentrated under reduced pressure. The residue was extracted with EtOAc (3×60 mL). The combined organic layers were washed with brine (3×30 mL), dried over Na₂SO₄, filtered and concentrated under reduced pressure. The residue was purified by column chromatography (SiO₂, Petroleum ether:Ethyl acetate=100:1 to 0:1) to give ethyl (R)-10-((7-(tert-butoxy)-7-oxoheptyl)oxy)-6-(tert-butyl)-2-oxo-6,7-dihydro-2H-pyrido[2′,1′:3,4]pyrazino[1,2-b]indazole-3-carboxylate (6) (1.6 g, 71.92% yield) as yellow solid.

### Procedure for Preparation of (R)-74(6-(tert-butyl)-3-(ethoxycarbonyl)-2-oxo-6,7-dihydro-2H-pyrido[2′,1′:3,4]pyrazino[1,2-b]indazol-10-yl)oxy)heptanoic Acid (7)

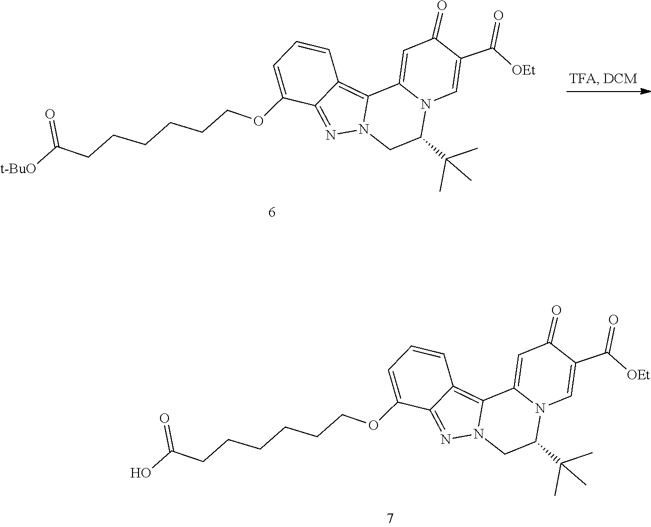 [SMILES: CCOC(=O)c1cn2c(cc1=O)-c1c3cccc(OCCCCCCC(=O)OC(C)(C)C)c3nn1C[C@H]2C(C)(C)C ; CCOC(=O)c1cn2c(cc1=O)-c1c3cccc(OCCCCCCC(=O)O)c3nn1C[C@H]2C(C)(C)C]

To a solution of ethyl (R)-10-((7-(tert-butoxy)-7-oxoheptyl)oxy)-6-(tert-butyl)-2-oxo-6,7-dihydro-2H-pyrido[2′,1′:3,4]pyrazino[1,2-b]indazole-3- carboxylate (6) (1.5 g, 2.65 mmol, 1 eq.) in DCM (10 mL) and TFA (5 mL) at 25° C. The mixture was stirred at 25° C. for 1 hr. LCMS showed the reaction was complete. The pH was adjusted to 7 with saturated NaHCO₃. The residue was extracted with DCM (3×15 mL). The combined organic layers were washed with brine (15 mL), dried over Na₂SO₄, filtered and concentrated under reduced pressure. The residue was purified by prep-HPLC (column: Welch Xtimate C18 250*70 mm*10 um; mobile phase: [water (NH₄HCO₃)-ACN]; B %: 10%-40%, 20 min) to give (R)-7-((6-(tert-butyl)-3-(ethoxycarbonyl)-2-oxo-6,7-dihydro-2H-pyrido[2′,1′:3,4]pyrazino[1,2-b]indazol-10-yl)oxy)heptanoic acid (7) (1.15 g, 84% yield, 98.76% purity) as a yellow solid.

¹H NMR (400 MHz, DMSO-d₆) δ ppm 8.52; (s, 1 H), 7.50; (d, J=8.4 Hz, 1 H), 7.18 (t, J=8.0 Hz, 1 H), 6.86-6.72; (m, 2 H), 5.14-5.00; (m, 2 H), 4.70; (d, J=4.4 Hz, 1 H), 4.24; (q, J=7.2 Hz, 2 H), 4.15-4.12; (m, 2 H), 2.19 (t,J=7.2 Hz, 2 H), 1.80; (q, J=6.8 Hz, 2 H), 1.57-1.42; (m, 4 H), 1.40-1.33; (m, 2 H), 1.28; (t, J=7.2 Hz, 3 H), 0.70; (s, 9 H).

### Procedure for Preparation of (R)-6-(tert-butyl)-10-((6-carboxyhexyl)oxy)-2-oxo-6,7-dihydro-2H-pyrido[2′,1′:3,4]pyrazino[1,2-b]indazole-3-carboxylic Acid (Compound I.2)

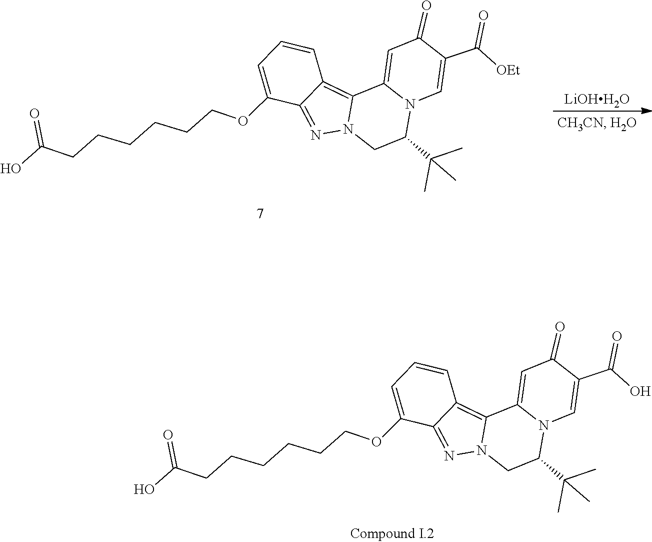 [SMILES: CCOC(=O)c1cn2c(cc1=O)-c1c3cccc(OCCCCCCC(=O)O)c3nn1C[C@H]2C(C)(C)C ; "LiOH·H2O" ; CC(C)(C)[C@@H]1Cn2nc3c(OCCCCCCC(=O)O)cccc3c2-c2cc(=O)c(C(=O)O)cn21]

To a solution of (R)-7-((6-(tert-butyl)-3-(ethoxycarbonyl)-2-oxo-6,7-dihydro-2H-pyrido[2′,1′:3,4]pyrazino[1,2-b]indazol-10-yl)oxy)heptanoic acid 7 (0.5 g, 981.18 umol, 1 eq) in CH₃CN (4 mL) and H₂O (2 mL) was added LiOH·H₂O (123.52 mg, 2.94 mmol, 3 eq). The mixture was stirred at 20° C. for 12 h. LCMS showed the reaction was complete. The mixture was filtered and concentrated under reduced pressure. The residue was purified by prep-HPLC (column: Phenomenex C18 80*40 mm*3 um; mobile phase: [water(NH₄HCO₃)-ACN]; B %: 1%-30%, 8 min) to give (R)-6-(tert-butyl)-10-((6-carboxyhexyl)oxy)-2-oxo-6,7-dihydro-2H-pyrido[2′,1′:3,4]pyrazino[1,2-b]indazole-3-carboxylic acid (Compound I.2) (137 mg, 281.9 umol, 29% yield, 99.23% purity) as a light yellow solid.

¹H NMR (400 MHz, DMSO-d₆) δ ppm 8.95; (s, 1 H), 7.63; (d, J=8.4 Hz, 1 H), 7.37-7.11; (m, 2 H), 6.82; (d, J=7.6 Hz, 1 H), 5.31-5.03; (m, 2 H), 4.96; (d, J=4.8 Hz, 1 H), 4.23-4.03; (m, 2 H), 2.21; (t, J=7.2 Hz, 2 H), 1.81; (quin, J=6.8 Hz, 2 H), 1.62-1.41; (m, 4 H), 1.41-1.30; (m, 2 H), 0.71; (s, 9 H).

### Compound I.3

### Synthetic Route:

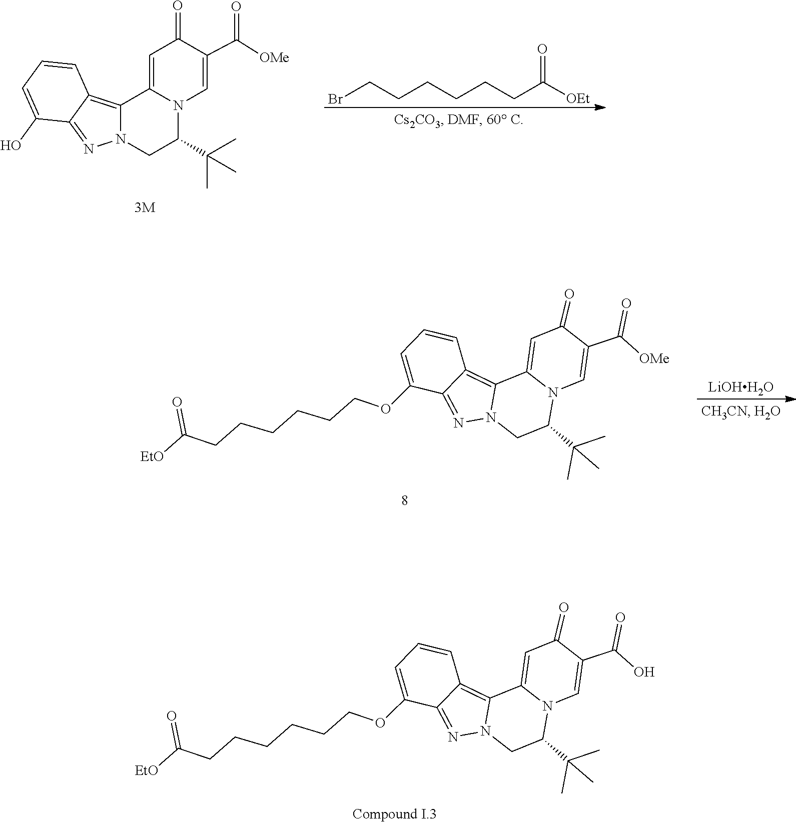 [SMILES: COC(=O)c1cn2c(cc1=O)-c1c3cccc(O)c3nn1C[C@H]2C(C)(C)C ; CCOC(=O)CCCCCCBr ; CCOC(=O)CCCCCCOc1cccc2c3n(nc12)C[C@@H](C(C)(C)C)n1cc(C(=O)OC)c(=O)cc1-3 ; "LiOH·H2O" ; CCOC(=O)CCCCCCOc1cccc2c3n(nc12)C[C@@H](C(C)(C)C)n1cc(C(=O)O)c(=O)cc1-3]

### Procedure for Preparation of Methyl (R)-6-(tert-butyl)-10-((7-ethoxy-7-oxoheptyl)oxy)-2-oxo-6,7-dihydro-2H-pyrido[2′,1′:3,4]pyrazino[1,2-b]indazole-3- carboxylate (8)

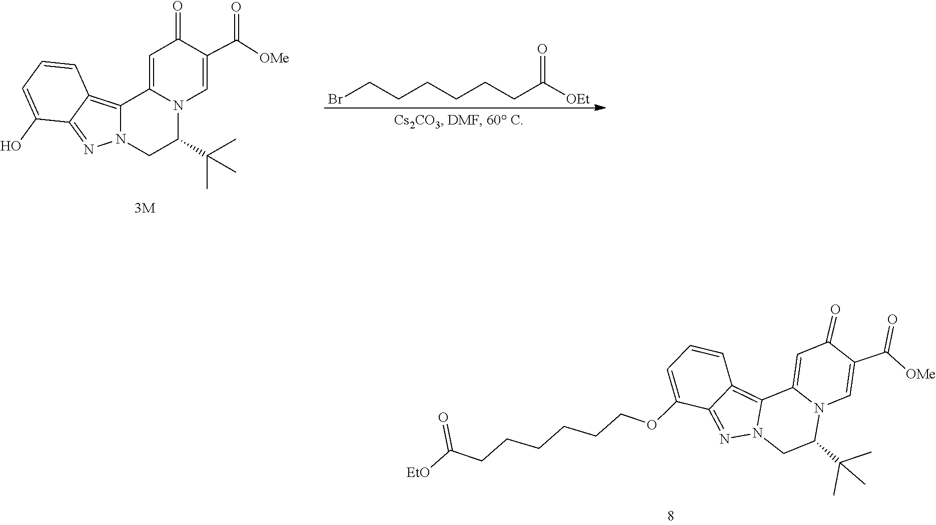 [SMILES: COC(=O)c1cn2c(cc1=O)-c1c3cccc(O)c3nn1C[C@H]2C(C)(C)C ; CCOC(=O)CCCCCCBr ; CCOC(=O)CCCCCCOc1cccc2c3n(nc12)C[C@@H](C(C)(C)C)n1cc(C(=O)OC)c(=O)cc1-3]

To a mixture of methyl (R)-6-(tert-butyl)-10-hydroxy-2-oxo-6,7-dihydro-2H-pyrido[2′,1′:3,4]pyrazino[1,2-b]indazole-3-carboxylate (3M) (6 g, 16.33 mmol) in DMF (60 mL) was added Cs₂CO₃ (15.96 g, 48.99 mmol) and ethyl-7-bromoheptanoate (5.03 g, 21.23 mmol). The mixture was stirred at 60° C. for 3 h. The mixture was monitored by TLC (SiO₂, Ethyl acetate:Methanol=10:1). The reaction mixture was quenched by addition of H₂O (180 mL), extracted with EtOAc (60 mL×3). The combined organic layers were washed with brine (50 mL×3), dried with Na₂SO₄ and concentrated under reduce pressure. The residue was purified by column chromatography (SiO₂, Petroleum ether:Ethyl acetate=1:0 to 0 : 1) to give methyl (R)-6-(tert-butyl)-10-((7-ethoxy-7-oxoheptyl)oxy)-2-oxo-6,7-dihydro-2H-pyrido[2′,1′:3,4]pyrazino[1,2-b]indazole-3-carboxylate (8) (5.9 g, 69% yield) as a yellow solid.

### Procedure for Preparation of (R)-6-(tert-butyl)-10-((7-ethoxy-7-oxoheptyl)oxy)-2-oxo-6,7-dihydro-2H-pyrido[2′,1′:3,4]pyrazino[1,2-b]indazole-3- carboxylic Acid (Compound I.3)

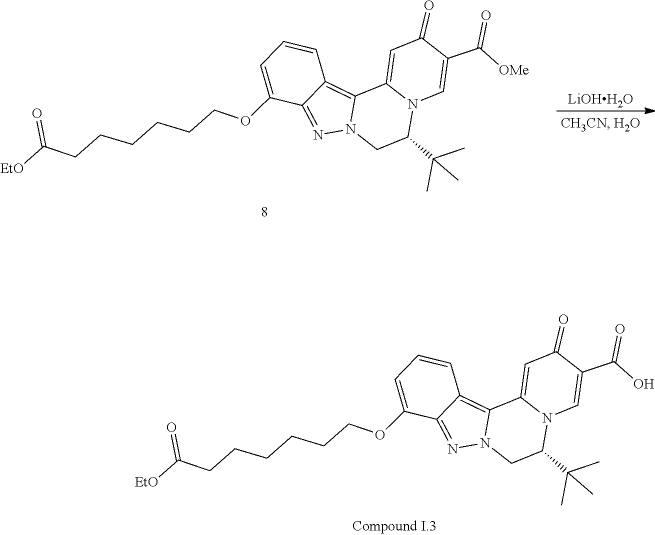 [SMILES: CCOC(=O)CCCCCCOc1cccc2c3n(nc12)C[C@@H](C(C)(C)C)n1cc(C(=O)OC)c(=O)cc1-3 ; "LiOH·H2O" ; CCOC(=O)CCCCCCOc1cccc2c3n(nc12)C[C@@H](C(C)(C)C)n1cc(C(=O)O)c(=O)cc1-3]

A mixture of methyl (R)-6-(tert-butyl)-10-((7-ethoxy-7-oxoheptyl)oxy)-2-oxo-6,7-dihydro-2H-pyrido[2′,1′:3,4]pyrazino[1,2-b]indazole-3-carboxylate (8) (5.9 g, 11.27 mmol) in H₂O (60 mL) and CH₃CN (60 mL) was added LiOH·H₂O (472.83 mg, 11.27 mmol) and stirred at 20° C. for 2 h. The mixture was monitored by LCMS. The mixture was adjusted to pH=4 with diluted HCl (1N) and extracted with a solution of 5% EtOH in DCM (50 mL×3). The combined organic layers were washed with brine (50 mL), dried over Na₂SO₄, filtered and concentrated under reduced pressure. The mixture was purified by prep-HPLC (column: Welch Xtimate C18 250*70 mm #10 um; mobile phase: [water(NH₄HCO₃)-ACN]; B %: 33%-63%, 20 min) to give (R)-6-(tert-butyl)-10-((7-ethoxy-7-oxoheptyl)oxy)-2-oxo-6,7-dihydro-2H-5 pyrido[2′,1′:3,4]pyrazino[1,2-b]indazole-3-carboxylic acid (Compound I.3) (2.5 g, 98% purity) as a yellow solid.

¹H NMR (400 MHz, DMSO-d₆) δ ppm 8.95; (s, 1 H), 7.63; (d, J=8.4 Hz, 1 H), 7.35-7.17; (m, 2 H), 6.82; (d, J=7.6 Hz, 1 H), 5.28-5.05; (m, 2 H), 4.96; (d, J=4.4 Hz, 1 H), 4.16; (t, J=5.6 Hz, 2 H), 4.04; (q, J=7.2 Hz, 2 H), 2.30; (t, J=7.2 Hz, 2 H), 1.89-1.78; (m, 2 H), 1.62-1.42; (m, 4 H), 1.41-1.31; (m, 2 H), 1.17; (t, J=7.2 Hz, 3 H), 0.71; (s, 9 H).

Mass-Spectrum calculated: 510.3, found: [M+H]⁺=510.3.

### Compound I.4

### Synthetic Route:

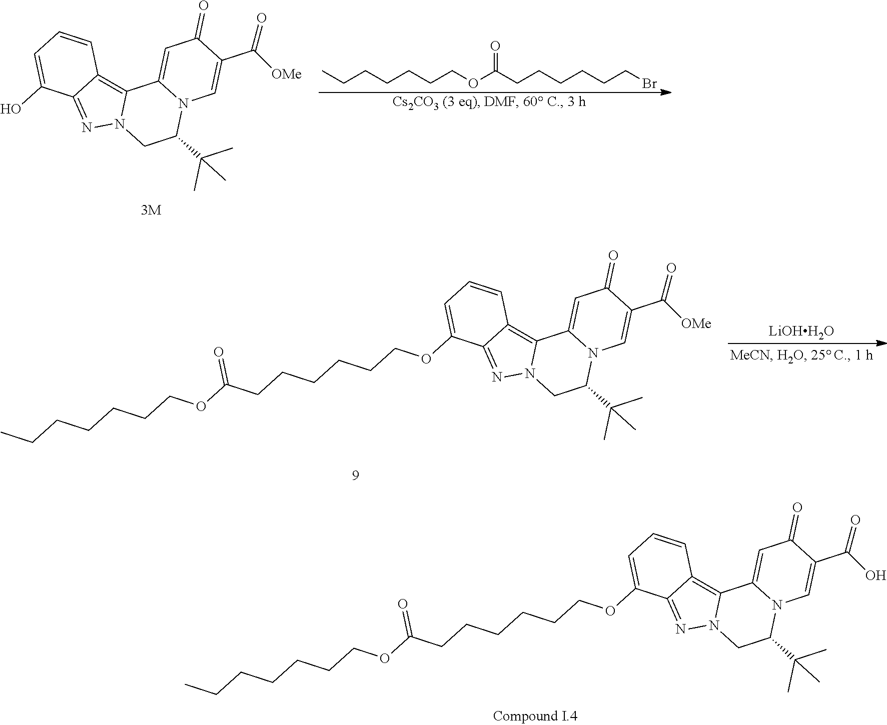

### Procedure for Preparation of Methyl (R)-6-(tert-butyl)-10-((7-(heptyloxy)-7-oxoheptyl)oxy)-2-oxo-6,7-dihydro-2H-pyrido[2′,1′:3,4]pyrazino[1,2-b]indazole-3-carboxylate (9)

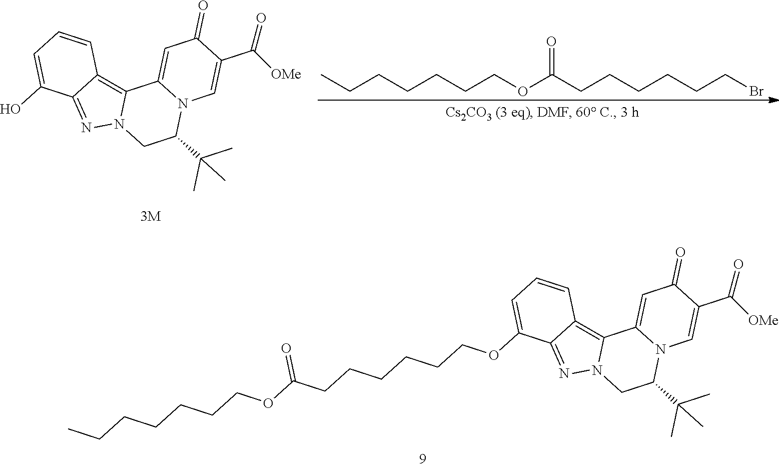

The reaction was split into two equal batches. In each of them, to a mixture of methyl (R)-6-(tert-butyl)-10-hydroxy-2-oxo-6,7-dihydro-2H-pyrido[2′,1′:3,4]pyrazino[1,2-b]indazole-3-carboxylate (3M) (250 mg, 0.680 mmol) in DMF (1 mL) was added Cs₂CO₃ (665 mg, 2.04 mmol) and stirred at 60° C. for 10 min. Then the mixture was added heptyl 7-bromoheptanoate (313.6 mg, 1.02 mmol, 1.5 eq) and stirred at 60° C. for 3 hrs. The mixture was monitored by LCMS. Two batches were combined for working up. The reaction mixture was quenched by addition of H₂O (3 mL) and extracted with EtOAc (10 mL×3). The combined organic layers were washed with brine (10 mL×3), dried with Na₂SO₄ and concentrated under reduce pressure. The residue was purified by column chromatography (SiO₂, Ethyl acetate: Methanol=10:1) to give methyl (R)-6-(tert-butyl)-10-((7-(heptyloxy)-7-oxoheptyl)oxy)-2-oxo-6,7-dihydro-2H-pyrido[2′,1′:3,4]pyrazino[1,2-b]indazole-3-carboxylate (9) (400 mg, crude) as a yellow solid.

### Procedure for Preparation (R)-6-(tert-butyl)-10-((7-(heptyloxy)-7-oxoheptyl)oxy)-2-oxo-6,7-dihydro-2H-pyrido[2′,1′:3,4]pyrazino[1,2-b]indazole-3- carboxylic Acid (Compound I.4)

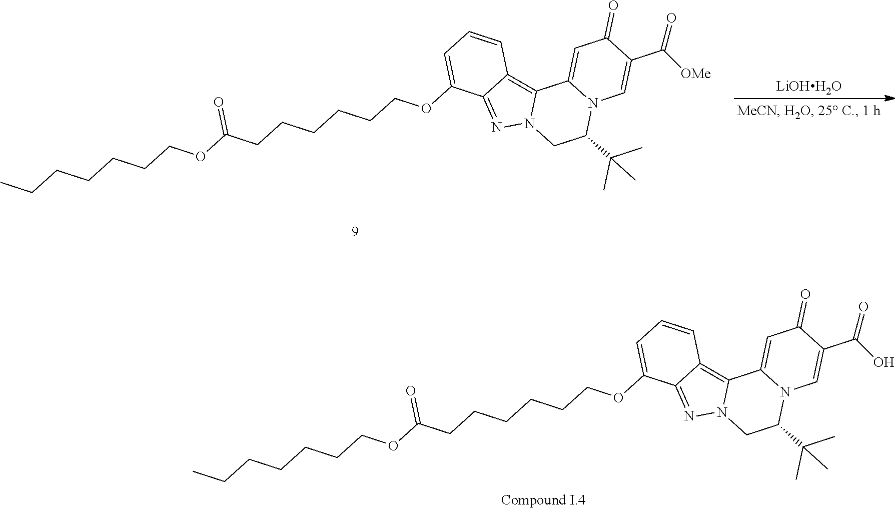 [SMILES: CCCCCCCOC(=O)CCCCCCOc1cccc2c3n(nc12)C[C@@H](C(C)(C)C)n1cc(C(=O)OC)c(=O)cc1-3 ; "LiOH·H2O" ; CCCCCCCOC(=O)CCCCCCOc1cccc2c3n(nc12)C[C@@H](C(C)(C)C)n1cc(C(=O)O)c(=O)cc1-3]

To a mixture of methyl (R)-6-(tert-butyl)-10-((7-(heptyloxy)-7-oxoheptyl)oxy)-2-oxo-6,7-dihydro-2H-pyrido[2′,1′:3,4]pyrazino[1,2-b]indazole-3- carboxylate (9) (350 mg, 0.589 mmol) in MeCN (3.5 mL) and H₂O (3.5 mL) was added LiOH·H₂O (25 mg, 0.589 mmol) and the reaction mixture was stirred at 25° C. for 2 hrs. The mixture was monitored by LCMS. The mixture was added 1N HCl to pH=4 and extracted with a solution of 5% EtOH in DCM (5 mL×3). The combined organic layer was washed with brine (10 mL), dried with Na₂SO₄ and concentrated under reduced pressure. The residue was purified by prep-HPLC (column: Phenomenex Luna C18 75*30 mm*3 um; mobile phase: [water(FA)-ACN]; B %: 50%-90%, 8 min). After lyophilization, the residue was repurified by prep-HPLC (column: Waters Xbridge Prep OBD C18 150*40 mm*10 um; mobile phase: [water(NH₄HCO₃)-ACN]; B %: 40%-95%, 8 min) to give (R)-6-(tert-butyl)-10-((7-(heptyloxy)-7-oxoheptyl)oxy)-2-oxo-6,7-dihydro-2H-pyrido[2′,1′:3,4]pyrazino[1,2-b]indazole-3-carboxylic acid (Compound I.4) (60 mg, purity: 98.7%) as a yellow solid.

¹H NMR (400 MHz, DMSO-d₆) δ ppm 8.95; (s, 1 H), 7.63; (d, J=8.4 Hz, 1 H), 7.32-7.18; (m, 2 H), 6.82; (d, J=7.6 Hz, 1 H), 5.27-5.04; (m, 2 H), 4.97; (d, J=4.4 Hz, 1 H), 4.16; (t, J=5.6 Hz, 2 H), 3.99; (t, J=6.8 Hz, 2 H), 2.30; (t, J=7.2 Hz, 2 H), 1.89-1.78; (m, 2 H), 1.65-1.42; (m, 6 H), 1.41-1.32; (m, 2 H), 1.31-1.18; (m, 8 H), 0.90-0.79; (m, 3 H), 0.71; (s, 9 H).

Mass-Spectrum calculated: 579.3, found: [M+H]⁺=580.3.

### Compound I.5

### Procedure for Preparation of (R)-6-(tert-butyl)-10-((7-isopropoxy-7-oxoheptyl)oxy)-2-oxo-6,7-dihydro-2H-pyrido[2′,1′:3,4]pyrazino[1,2-b]indazole-3- carboxylic Acid (Compound I.5)

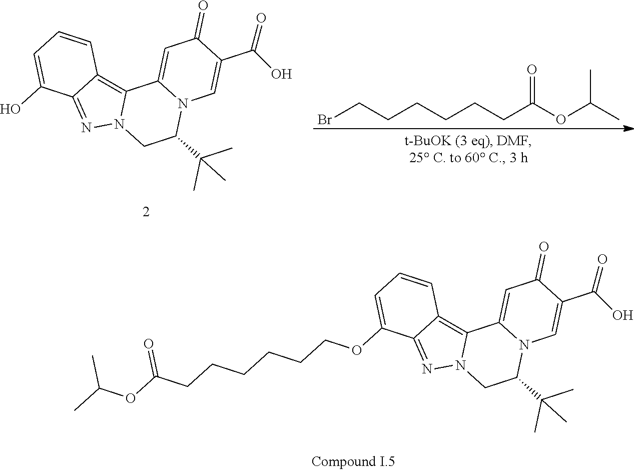 [SMILES: CC(C)(C)[C@@H]1Cn2nc3c(O)cccc3c2-c2cc(=O)c(C(=O)O)cn21 ; CC(C)OC(=O)CCCCCCBr ; CC(C)OC(=O)CCCCCCOc1cccc2c3n(nc12)C[C@@H](C(C)(C)C)n1cc(C(=O)O)c(=O)cc1-3]

To a solution of (R)-6-(tert-butyl)-10-hydroxy-2-oxo-6,7-dihydro-2H-pyrido[2′,1′:3,4]pyrazino[1,2-b]indazole-3-carboxylic acid (2) (500 mg, 1.41 mmol, 1 eq) and t-BuOK (476.31 mg, 4.24 mmol, 3 eq) in DMF (7.5 mL) was added isopropyl 7-bromoheptanoate (284 mg, 1.13 mmol, 0.8 eq) in DMF (2.5 mL). The mixture was stirred at 60° C. for 3 h. TLC (Ethyl acetate:methanol=10:1, R_{f}=0.2) showed the reaction was completed. The mixture was poured into the diluted HCl (0.1M, 70 mL) and filtered. The filtrate was extracted with EtOAc (20 mL×3). The combined organic layers were washed with brine (20 mL×2), dried over Na₂SO₄, filtered and concentrated under reduced pressure. The mixture was purified by prep-HPLC (Waters Xbridge Prep OBD C18 150*40 mm*10 um; mobile phase: [water(NH₄HCO₃)-ACN]; B %: 35%-65%, 8 min) to give (R)-6-(tert-butyl)-10-((7-isopropoxy-7-oxoheptyl)oxy)-2-oxo-6,7-dihydro-2H-pyrido[2′,1′:3,4]pyrazino[1,2-b]indazole-3-carboxylic acid (Compound I.5) (58.9 mg, 111 umol, 8% yield, 99% purity) as a yellow solid.

¹H NMR (400 MHz, DMSO-d₆) δ ppm 8.96; (s, 1 H), 7.64; (d, J=8.4 Hz, 1 H), 7.28-7.21; (m, 2 H), 6.82; (d, J=7.6 Hz, 1 H), 5.23-5.07; (m, 2 H), 4.97; (d, J=4.8 Hz, 1 H), 4.88; (td, J=6.4, 12.4 Hz, 1 H), 4.19-4.13; (m, 2 H), 2.26; (t, J=7.2 Hz, 2 H), 1.86-1.77; (m, 2 H), 1.60-1.43; (m, 4 H), 1.41-1.32; (m, 2 H), 1.17; (d, J=6.4 Hz, 6 H), 0.71; (s, 9 H).

Mass-Spectrum calculated: 523.2, found: [M+H]⁺=524.3.

### Compound I.6

### Synthetic Route:

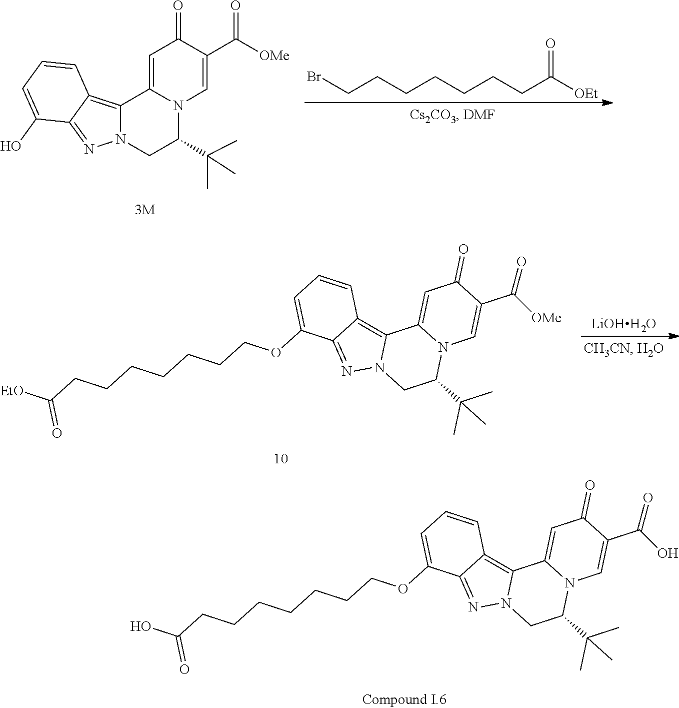

Procedure for Preparation of Methyl (R)-6-(tert-butyl)-10-((8-ethoxy-8-oxooctyl)oxy)-2-oxo-6,7-dihydro-2H-pyrido[2′,1′:3,4]pyrazino[1,2-b]indazole-3- carboxylate (10)

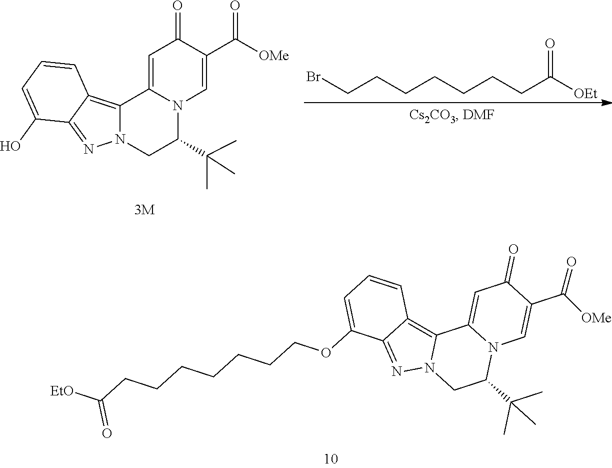 [SMILES: COC(=O)c1cn2c(cc1=O)-c1c3cccc(O)c3nn1C[C@H]2C(C)(C)C ; CCOC(=O)CCCCCCCBr ; CCOC(=O)CCCCCCCOc1cccc2c3n(nc12)C[C@@H](C(C)(C)C)n1cc(C(=O)OC)c(=O)cc1-3]

To a mixture of methyl (R)-6-(tert-butyl)-10-hydroxy-2-oxo-6,7-dihydro-2H-pyrido[2′,1′:3,4]pyrazino[1,2-b]indazole-3-carboxylate (3M) (1 g, 2.62 mmol) in DMF (10 mL) was added Cs₂CO₃ (2.56 g, 7.87 mmol) and ethyl 8-bromooctanoate (856.02 mg, 3.41 mmol) at 25° C. The mixture was stirred at 60° C. for 3 h. The mixture was monitored by LCMS. The mixture was quenched by addition of H₂O (30 mL) and extracted with EtOAc (15 mL×3). The combined organic layer was washed with brine (10 mL×3), dried with Na₂SO₄ and concentrated under reduce pressure. The residue was purified by column chromatography (SiO₂, Petroleum ether:Ethyl acetate=1:0 to 0:1) to give methyl (R)-6-(tert-butyl)-10-((8-ethoxy-8-oxooctyl)oxy)-2-oxo-6,7-dihydro-2H-pyrido[2′,1′:3,4]pyrazino[1,2-b]indazole-3-carboxylate (10) (1.20 g, 72% yield) as a yellow solid.

### Procedure for Preparation of (R)-6-(tert-butyl)-10-((7-carboxyheptyl)oxy)-2-oxo-6,7-dihydro-2H-pyrido[2′,1′:3,4]pyrazino[1,2-b]indazole-3-carboxylic Acid (Compound I.6)

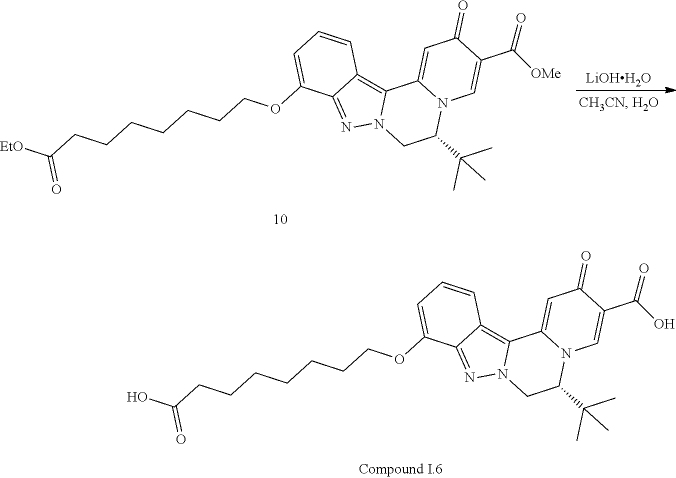

To a mixture of methyl (R)-6-(tert-butyl)-10-((8-ethoxy-8-oxooctyl)oxy)-2-oxo-6,7-dihydro-2H-pyrido[2′,1′:3,4]pyrazino[1,2-b]indazole-3-carboxylate (10) (1.20 g, 2.18 mmol) in CH₃CN (10 mL) and H₂O (10 mL) was added LiOH·H₂O (274 mg, 6.53 mmol) and stirred at 20° C. for 2 h. The mixture was monitored by LCMS. The mixture was added HCl (1N) to pH=4 and extracted with a solution of 5% EtOH in DCM (10 mL×3). The combined organic layer was washed with brine (10 mL), dried with Na₂SO₄ and concentrated under reduced pressure. The residue was purified by prep-HPLC (column: Waters Xbridge BEH C18 250*50 mm*10 um; mobile phase: [water(NH₄HCO₃)-ACN]; B %: 5%-45%, 10 min) to give (R)-6-(tert-butyl)-10-((7-carboxyheptyl)oxy)-2-oxo-6,7-dihydro-2H-pyrido[2′,1′:3,4]pyrazino[1,2-b]indazole-3-carboxylic acid (Compound I.6) (500 mg, 100% purity) as a white solid.

¹H NMR (400 MHz, DMSO-d₆) δ ppm 8.95; (s, 1 H), 7.63; (d, J=8.4 Hz, 1 H), 7.34-7.19; (m, 2 H), 6.83; (d, J=8.0 Hz, 1 H), 5.27-5.06; (m, 2 H), 4.96; (d, J=4.0 Hz, 1 H), 4.23-4.08; (m, 2 H), 2.19; (t, J=7.2 Hz, 2 H), 1.89-1.78; (m, 2 H), 1.58-1.40; (m, 4 H), 1.39-1.21; (m, 4 H), 0.71; (s, 9 H).

Mass-Spectrum calculated: 496.2, found: [M+H]⁺=496.3.

### Compound I.7

### Synthetic Route:

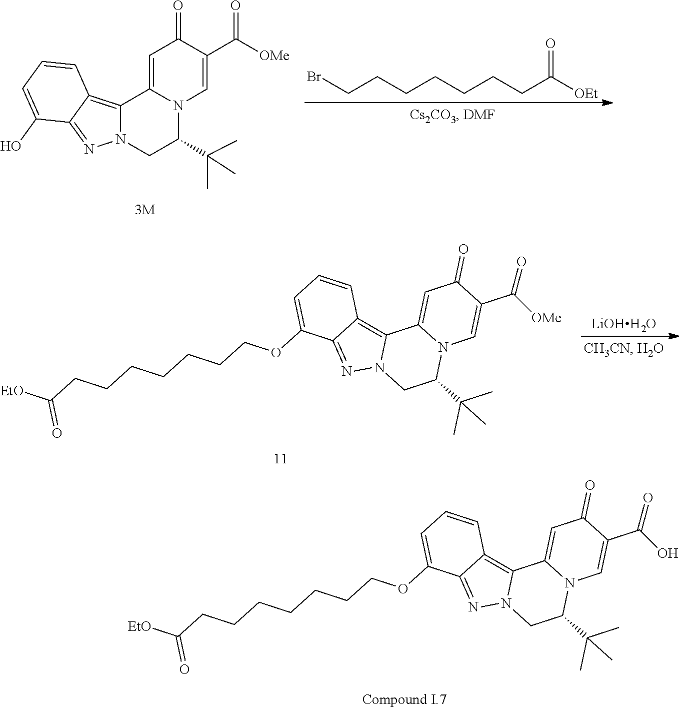

### Procedure for Preparation of Methyl (R)-6-(tert-butyl)-10-((8-ethoxy-8-oxooctyl)oxy)-2-oxo-6,7-dihydro-2H-pyrido[2′,1′:3,4]pyrazino[1,2-b]indazole-3- carboxylate (11)

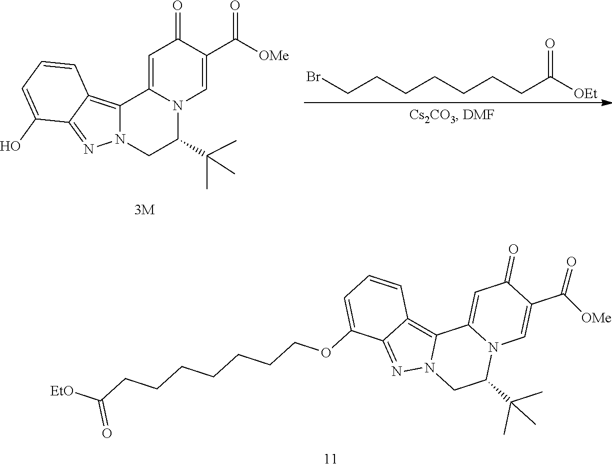 [SMILES: COC(=O)c1cn2c(cc1=O)-c1c3cccc(O)c3nn1C[C@H]2C(C)(C)C ; CCOC(=O)CCCCCCCBr ; CCOC(=O)CCCCCCCOc1cccc2c3n(nc12)C[C@@H](C(C)(C)C)n1cc(C(=O)OC)c(=O)cc1-3]

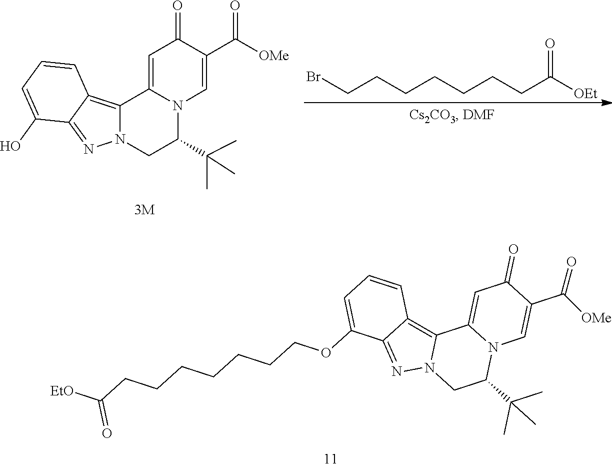 [SMILES: COC(=O)c1cn2c(cc1=O)-c1c3cccc(O)c3nn1C[C@H]2C(C)(C)C ; CCOC(=O)CCCCCCCBr ; CCOC(=O)CCCCCCCOc1cccc2c3n(nc12)C[C@@H](C(C)(C)C)n1cc(C(=O)OC)c(=O)cc1-3]

To a mixture of methyl (R)-6-(tert-butyl)-10-hydroxy-2-oxo-6,7-dihydro-2H-pyrido[2′,1′:3,4]pyrazino[1,2-b]indazole-3-carboxylate (3M) (5.00 g, 13.61 mmol) in DMF (50 mL) was added Cs₂CO₃ (13.30 g, 40.83 mmol) and ethyl 8-bromooctanoate (4.44 g, 17.69 mmol). The mixture was stirred at 60° C. for 3 h. The mixture was monitored by TLC (SiO₂, Ethyl acetate:Methanol=10:1, R_{f}=0.5). The reaction mixture was quenched by addition of H₂O (150 mL) and extracted with EtOAc (50 mL×3). The combined organic layer was washed with brine (50 mL×3), dried over Na₂SO₄ and concentrated under reduced pressure. The residue was purified by column chromatography (SiO₂, Petroleum ether:Ethyl acetate=1:0 to 0:1) to give methyl (R)-6-(tert-butyl)-10-((8-ethoxy-8-oxooctyl)oxy)-2-oxo-6,7-dihydro-2H-pyrido[2′,1′:3,4]pyrazino[1,2-b]indazole-3-carboxylate (11) (4.8 g, 65.6% yield) as a yellow solid.

### Procedure for Preparation of (R)-6-(tert-butyl)-10-((8-ethoxy-8-oxooctyl)oxy)-2-oxo-6,7-dihydro-2H-pyrido[2′,1′:3,4]pyrazino[1,2-b]indazole-3- carboxylic Acid (Compound I.7)

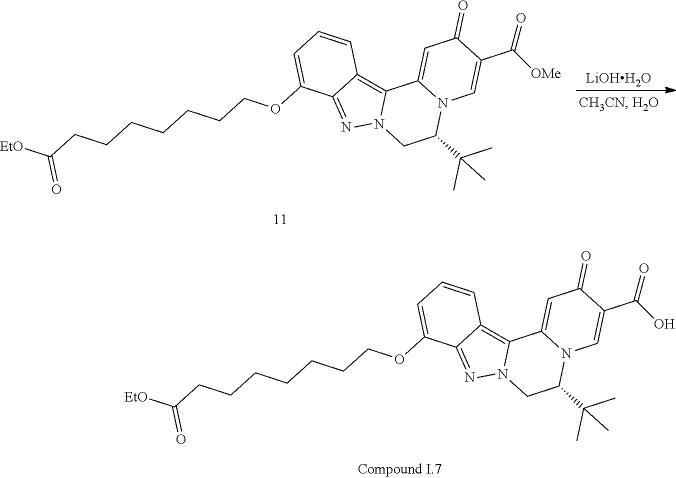

A mixture of methyl (R)-6-(tert-butyl)-10-((8-ethoxy-8-oxooctyl)oxy)-2-oxo-6,7-dihydro-2H-pyrido[2′,1′:3,4]pyrazino[1,2-b]indazole-3-carboxylate (11) (4.8 g, 8.93 mmol) in H₂O (50 mL) and MeCN (50 mL) was added LiOH·H₂O (374.61 mg, 8.93 mmol). The reaction mixture was stirred at 20° C. for 2 h. The mixture was monitored by LCMS. The mixture was quenched by addition of 1N HCl to pH=4, extracted with 5% EtOH in DCM (50 mL×3). The combined organic layers were washed with brine (50 mL), dried with Na₂SO₄, filtered and concentrated under reduced pressure. The residue was purified by prep-HPLC(column: Waters Xbridge BEH C18 250*70 mm*10 um; mobile phase: [water(NH₄HCO₃)-ACN]; B %: 30%-75%, 18 min) to give (R)-6-(tert-butyl)-10-((8-ethoxy-8-oxooctyl)oxy)-2-oxo-6,7-dihydro-2H-pyrido[2′,1′:3,4]pyrazino[1,2-b]indazole-3-carboxylic acid (Compound I.7) (2.80 g, 98% purity) as a yellow solid.

¹H NMR (400 MHz, DMSO-d₆) δ ppm 8.96; (s, 1 H), 7.63; (d, J=8.4 Hz, 1 H), 7.34-7.15; (m, 2 H), 6.83; (d, J=7.2 Hz, 1 H), 5.28-5.06; (m, 2 H), 4.97; (d, J=4.4 Hz, 1 H), 4.16; (t, J=6.0 Hz, 2 H), 4.04; (q, J=7.2 Hz, 2 H), 2.28; (t, J=7.2 Hz, 2 H), 1.81; (quin, J=6.8 Hz, 2 H), 1.59-1.41; (m, 4 H), 1.41-1.26; (m, 4 H), 1.17; (t, J=7.2 Hz, 3 H), 0.71; (s, 9 H).

Mass-Spectrum calculated: 523.3, found: [M+H]⁺=524.3.

### Compound I.8

### Synthetic Route:

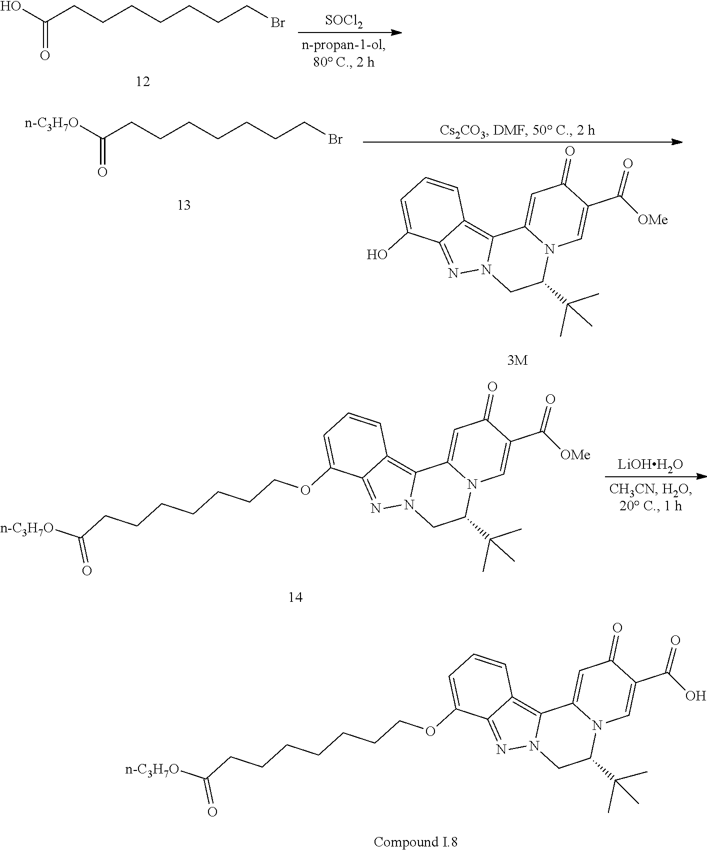

### Procedure for Preparation of propyl 8-bromooctanoate (13)

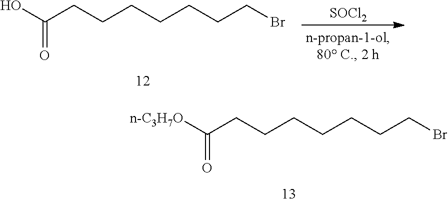

To a solution of 8-bromooctanoic acid (12) (3.00 g, 13.45 mmol, 1 eq) in propan-1-ol (30 mL) was added SOCl₂ (3.20 g, 26.89 mmol, 1.95 mL, 2 eq). The mixture was stirred at 80° C. for 2 h. TLC (Petroleum ether:Ethyl acetate=5:1, R_{f}=0.5) indicated the reaction was completed. After removed the solvent under reduced pressure, the residue was dissolved into EtOAc (50 mL) and water (50 mL). The organic layer was separated, dried over Na₂SO₄, filtered and concentrated under reduced pressure. The residue was purified by column chromatography (SiO₂, Petroleum ether:Ethyl acetate=50:1 to 20:1) to give propyl 8-bromooctanoate (3.37 g, 12.71 mmol, 94% yield) as a colorless oil.

¹H NMR (400 MHz, CDCl₃) 6 ppm 4.03; (td, J=6.4, 0.8 Hz, 2 H), 3.40; (td, J=6.8, 0.8 Hz, 2 H), 2.35-2.26; (m, 2 H), 1.93-1.79; (m, 2 H), 1.68-1.59; (m, 4 H), 1.49-1.39; (m, 2 H), 1.34; (dt, J=6.8, 3.2 Hz, 4 H), 0.94; (td, J=7.6, 1.2 Hz, 3 H).

### Procedure for Preparation of Methyl (R)-6-(tert-butyl)-2-oxo-10-((8-oxo-8-propoxyoctyl)oxy)-6,7-dihydro-2H-pyrido[2′,1′:3,4]pyrazino[1,2-b]indazole-3- carboxylate (14)

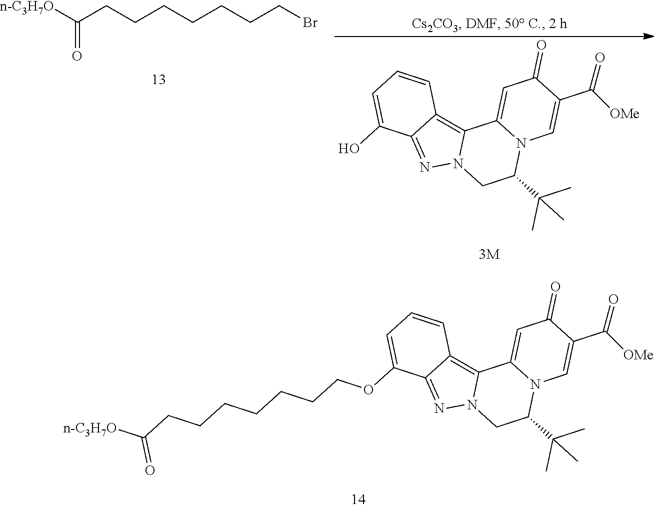

To a solution of methyl (R)-6-(tert-butyl)-10-hydroxy-2-oxo-6,7-dihydro-2H-pyrido[2′,1′:3,4]pyrazino[1,2-b]indazole-3-carboxylate (3M) (2.5 g, 6.80 mmol, 1 eq) in DMF (20 mL) was added Cs₂CO₃ (7.76 g, 23.82 mmol, 3.5 eq) and propyl 8-bromooctanoate (13) (1.98 g, 7.49 mmol, 1.1 eq). The mixture was stirred at 50° C. for 2 h. LCMS indicated the reaction was completed. The reaction mixture was poured into water (30 mL), extracted with EtOAc (50 mL×2). The organic layer was washed with brine (30 mL×2), dried over Na₂SO₄, filtered and concentrated under reduced pressure. The residue was purified by column chromatography (SiO₂, Petroleum ether:Ethyl acetate=2:1 to 0:1) to give methyl (R)-6-(tert-butyl)-2-oxo-10-((8-oxo-8-propoxyoctyl)oxy)-6,7-dihydro-2H-pyrido[2′,1′:3,4]pyrazino[1,2-b]indazole-3-carboxylate (14) (2.5 g, 4.53 mmol, 66% yield) as a white solid.

¹H NMR (400 MHz, CDCl₃) δ ppm 8.30; (s, 1 H), 7.43; (d, J=8.4 Hz, 1 H), 7.20; (t, J=8.0 Hz, 1 H), 7.08; (s, 1 H), 6.67; (d, J=7.6 Hz, 1 H), 5.17; (br d, J=14.8 Hz, 1 H), 4.90; (br d, J=11.6 Hz, 1 H), 4.20; (t, J=6.4 Hz, 2 H), 4.10; (br s, 1 H), 4.03; (t, J=6.4 Hz, 2 H), 3.95; (s, 3 H) 2.31; (t, J=7.6 Hz, 2 H), 1.97; (quintet, J=7.2 Hz, 2 H), 1.68-1.60; (m, 4 H), 1.57-1.49; (m, 2 H), 1.45-1.35; (m, 4 H), 0.94; (t, J=7.6 Hz, 3 H), 0.84; (s, 9 H).

### Procedure for Preparation of (R)-6-(tert-butyl)-2-oxo-10-((8-oxo-8-propoxyoctyl)oxy)-6,7-dihydro-2H-pyrido[2′,1′:3,4]pyrazino[1,2-b]indazole-3- carboxylic Acid (Compound I.8)

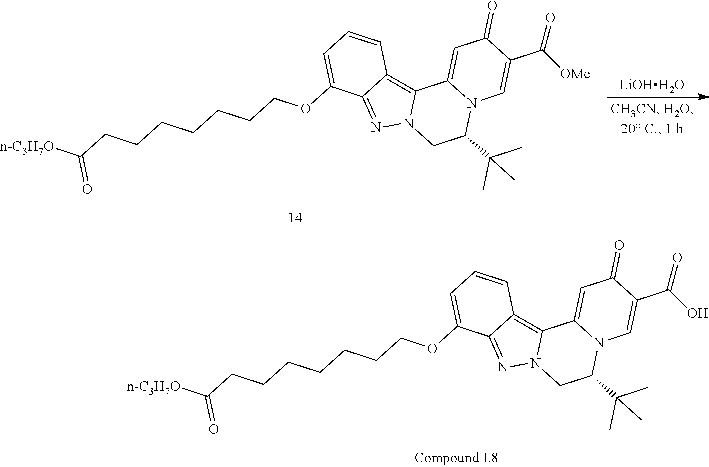

To a solution of methyl (R)-6-(tert-butyl)-2-oxo-10-((8-oxo-8-propoxyoctyl)oxy)-6,7-dihydro-2H-pyrido[2′,1′:3,4]pyrazino[1,2-b]indazole-3-carboxylate (14) (2.5 g, 4.53 mmol, 1 eq) in CH₃CN (12 mL) and H₂O (12 mL) was added LiOH·H₂O (247.22 mg, 5.89 mmol, 1.3 eq). The mixture was stirred at 20° C. for 1 h. LCMS indicated the reaction was completed. The reaction was poured into water (30 mL), adjusted the pH to 6-7 using 1M HCl. The mixture was extracted with EtOAc (20 mL×2). The organic layer was washed with brine (10 mL×2), dried over Na₂SO₄, filtered and concentrated under reduced pressure. The residue was purified by flash silica gel chromatography (column: Welch Xtimate C18 250*70 mm #10 um; mobile phase: [water(NH₄HCO₃)-ACN]; B %: 40%-65%, 20 min) to give (R)-6-(tert-butyl)-2-oxo-10-((8-oxo-8-propoxyoctyl)oxy)-6,7-dihydro-2H-pyrido[2′,1′:3,4]pyrazino[1,2-b]indazole-3-carboxylic acid (Compound I.8) (1.24 g, 2.31 mmol, 51% yield) as a white solid.

¹H NMR (400 MHz, DMSO-d₆) δ ppm 8.95; (s, 1 H), 7.62; (br d, J=8.4 Hz, 1 H), 7.25; (br d, J=7.6 Hz, 2 H), 6.82; (d, J=7.6 Hz, 1 H), 5.04-5.27; (m, 2 H), 4.97; (br s, 1 H), 4.15; (br t, J=5.2 Hz, 2 H), 3.95; (t, J=6.8 Hz, 2 H), 2.29; (t, J=7.6 Hz, 2 H), 1.81; (quin, J=6.8 Hz, 2 H), 1.62-1.51; (m, 4 H), 1.50-1.41; (m, 2 H), 1.40-1.26; (m, 4 H), 0.86; (t, J=7.6 Hz, 3 H), 0.70; (s, 9 H).

### Compound I.9

### Synthetic Route:

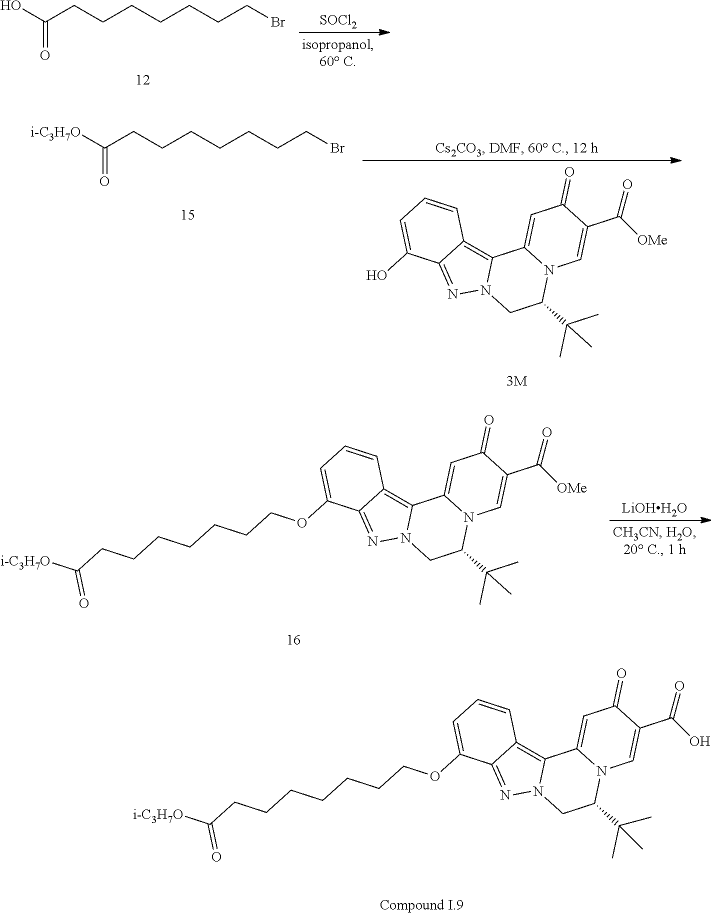

### Procedure for Preparation of isopropyl 8-bromooctanoate (15)

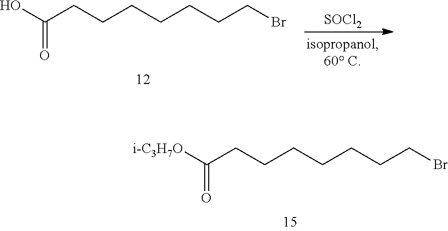

To a solution of 8-bromooctanoic acid (12) (3.5 g, 15.69 mmol) in i-PrOH (35 mL) was added SOCl₂ (3.73 g, 31.38 mmol, 2.28 mL). The mixture was stirred at 60° C. for 12 h. The reaction was monitored by TLC (Petroleum ether:Ethyl acetate=10:1, R_{f}=0.55). The reaction mixture was quenched by addition H₂O (25 mL) at 25° C., extracted with EtOAc (30 mL×3). The combined organic extracts were washed with brine, dried over Na₂SO₄, filtered and concentrated under reduced pressure. The residue was purified by column chromatography (SiO₂, Petroleum ether:Ethyl acetate=1:0 to 5:1) to give isopropyl 8-bromooctanoate (15) (5.7 g, 21.49 mmol, 95.91% yield) as a colorless oil.

¹H NMR (400 MHz, CDCl₃) δ=5.01-4.95; (m, 1H), 3.52-3.49; (m, 1H), 3.39-3.36; (m, 1H), 2.26-2.22; (m, 2H), 1.83-1.81; (m, 1H), 1.78-1.71; (m, 1H), 1.59-1.57; (m, 2H), 1.45-1.38; (m, 2H), 1.33-1.30; (m, 4H), 1.29-1.20; (m, 6H),

### Procedure for Preparation of Methyl (R)-6-(tert-butyl)-10-((8-isopropoxy-8-oxooctyl)oxy)-2-oxo-6,7-dihydro-2H-pyrido[2′,1′:3,4]pyrazino[1,2-b]indazole-3-carboxylate (16)

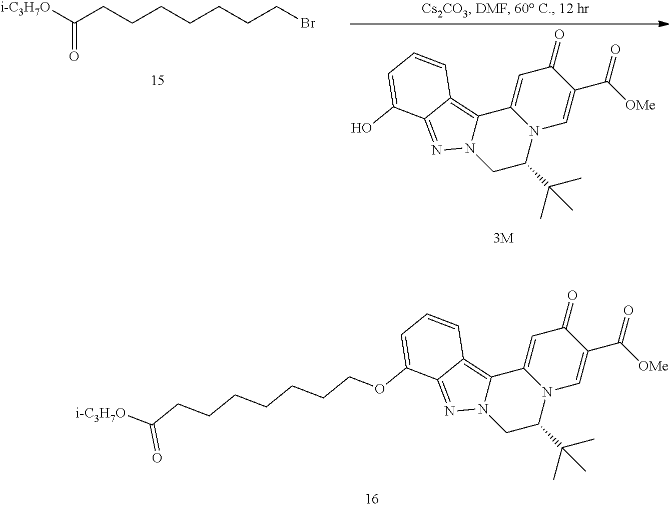

To a solution of methyl (R)-6-(tert-butyl)-10-hydroxy-2-oxo-6,7-dihydro-2H-pyrido[2′,1′:3,4]pyrazino[1,2-b]indazole-3-carboxylate (3M) (3.52 g, 9.57 mmol) in DMF (35 mL) was added Cs₂CO₃ (9.36 g, 28.72 mmol) and isopropyl 8-bromooctanoate (15) (3.3 g, 12.44 mmol), The mixture was stirred at 60° C. for 12 h. The reaction was monitored by TLC (Petroleum ether:THF=10:1, R_{f}=0.33). The reaction mixture was quenched by H₂O (30 mL) at 25° C., extracted with EtOAc (30 mL×3). The combined organic layers were washed with brine (25 mL), dried over Na₂SO₄, filtered and concentrated under reduced pressure. The residue was purified by column chromatography on silica gel (THF:Petroleum ether=1:1 to 1:0) to give methyl (R)-6-(tert-butyl)-10-((8-isopropoxy-8-oxooctyl)oxy)-2-oxo-6,7-dihydro-2H-pyrido[2′,1′:3,4]pyrazino[1,2-b]indazole-3-carboxylate (16) (3.56 g, 6.45 mmol, 67% yield) as a yellow solid.

Mass-Spectrum calc.: 551.3, found: [M+H]⁺=552.3

### Procedure for Preparation of (R)-6-(tert-butyl)-10-((8-isopropoxy-8-oxooctyl)oxy)-2-oxo-6,7-dihydro-2H-pyrido[2′,1′:3,4]pyrazino[1,2-b]indazole-3- carboxylic Acid (Compound I.9)

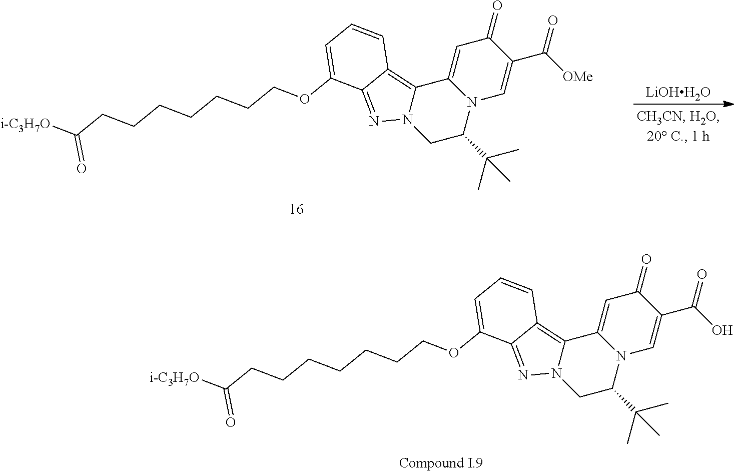

To a solution of methyl (R)-6-(tert-butyl)-10-((8-isopropoxy-8-oxooctyl)oxy)-2-oxo-6,7-dihydro-2H-pyrido[2′,1′:3,4]pyrazino[1,2-b]indazole-3-carboxylate (16) (2.2 g, 3.99 mmol,) in MeCN (20 mL) and H₂O (20 mL) was added LiOH·H₂O (200.81 mg, 4.79 mmol), stirred at 25° C. for 1 hr. The reaction was monitored by LCMS. The mixture was added 1N HCl to pH =4 and extracted with 5% EtOH in DCM (3×50 mL). The combined organic layer was washed with brine (50 mL), dried with Na₂SO₄ and concentrated under reduced pressure. The residue was purified by prep-HPLC (column: Agela DuraShell C18 250*70 mm*10 um; mobile phase: [water(NH₄HCO₃)-ACN]; B %: 40%-70%, 20 min) to give (R)-6-(tert-butyl)-10-((8-isopropoxy-8-oxooctyl)oxy)-2-oxo-6,7-dihydro-2H-pyrido[2′,1′:3,4]pyrazino[1,2-b]indazole-3-carboxylic acid (Compound I.9) (3.20 g, 5.95 mmol, 94% yield) as a yellow solid.

¹H NMR (400 MHz, DMSO-d₆) δ ppm 8.95; (s, 1 H), 7.63; (d, J=8.4 Hz, 1 H), 7.26-7.22; (m, 2 H), 6.83-6.80; (m, 1 H), 5.17-5.12; (m, 2 H), 4.97-4.96; (m, 1 H), 4.89-4.86; (m, 1 H), 4.17-4.15; (m, 2 H), 2.27-2.23; (m, 2 H), 1.89-1.75; (s, 2 H), 1.55-1.45; (m, 4 H), 1.34-1.32; (m, 4 H), 1.17-1.16; (m, 6 H), 0.71; (s, 9 H). Mass spectrum calc: 537.3, found: [M+H]⁺=538.3.

### Compound I.10

### Synthetic Route:

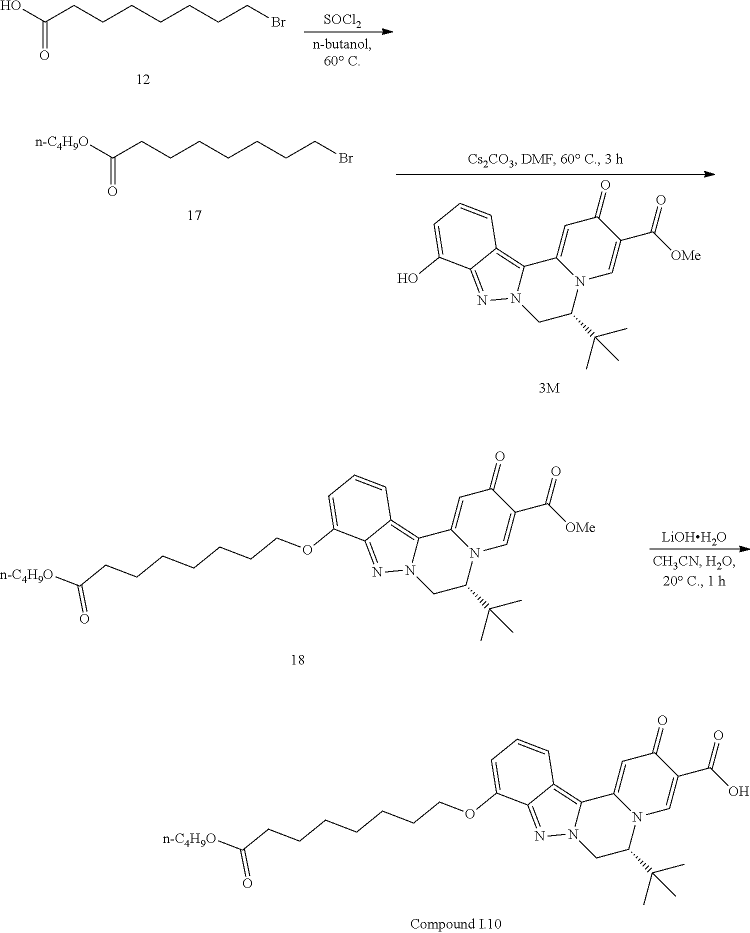

### Procedure for Preparation of butyl 8-bromooctanoate (17)

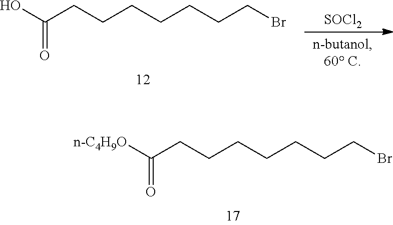

A mixture of 8-bromooctanoic acid (12) (10 g, 44.82 mmol) in n-BuOH (100 mL) was added SOCl 2 (16.00 g, 134.46 mmol) and stirred at 60° C. for 3 h. The mixture was monitored by TLC (SiO₂, Petroleum ether:Ethyl acetate=5:1, R_{f}=0.5). The mixture was concentrated under reduced pressure. The mixture was purified by column chromatography (SiO₂, Petroleum ether:Ethyl acetate=1:0 to 5:1) to give butyl 8-bromooctanoate (17) (12 g, crude) as a colorless oil.

### Procedure for Preparation of Methyl (R)-10-((8-butoxy-8-oxooctyl)oxy)-6-(tert-butyl)-2-oxo-6,7-dihydro-2H-pyrido[2′,1′:3,4]pyrazino[1,2-b]indazole-3- carboxylate (18)

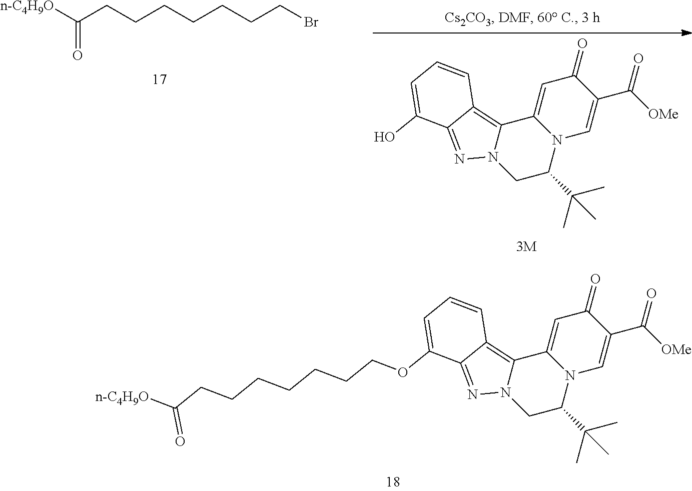

A mixture of methyl (R)-6-(tert-butyl)-10-hydroxy-6,7-dihydro-2H-pyrido[2′,1′:3,4]pyrazino[1,2-b]indazole-3-carboxylate (3M) (3.5 g, 9.53 mmol) in DMF (35 mL) was added Cs₂CO₃ (9.31 g, 28.58 mmol, 3 eq) and butyl 8-bromooctanoate (17) (3.46 g, 12.38 mmol, 1.3 eq) at 20° C. The mixture was stirred at 60° C. for 3 h. The mixture was monitored by LCMS. The mixture was quenched by addition of H₂O (100 mL) and extracted with EtOAc (50 mL×3). The combined organic layer was washed with brine (50 mL x 3), dried over Na₂SO₄ and concentrated under reduced pressure. The residue was purified by column chromatography (SiO₂, Petroleum ether:Ethyl acetate=1:0 to 0:1) to give methyl (R)-10-((8-butoxy-8-oxooctyl)oxy)-6-(tert-butyl)-2-oxo-6,7-dihydro-2H-pyrido[2′,1′:3,4]pyrazino[1,2-b]indazole-3-carboxylate (18) (2.5 g, 45.25% yield) as a yellow solid.

### Procedure for Preparation of (R)-10-((8-butoxy-8-oxooctyl)oxy)-6-(tert-butyl)-2-oxo-6,7-dihydro-2H-pyrido[2′,1′:3,4]pyrazino[1,2-b]indazole-3- carboxylic Acid (Compound I.10)

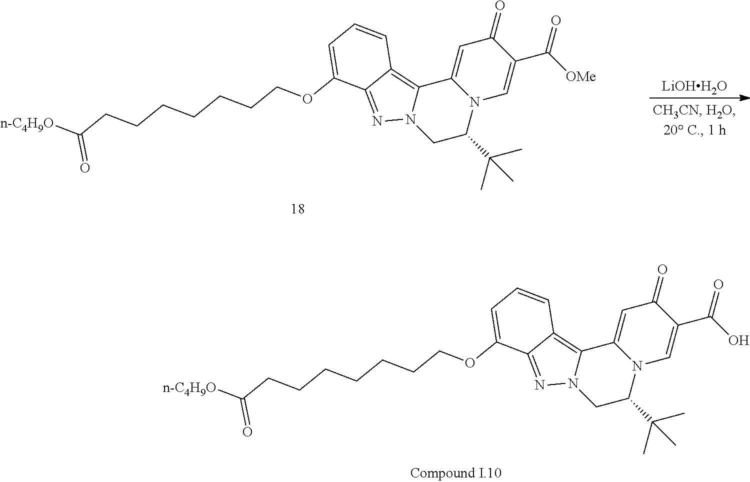

A mixture of methyl (R)-10-((8-butoxy-8-oxooctyl)oxy)-6-(tert-butyl)-2-oxo-6,7-dihydro-2H-pyrido[2′,1′:3,4]pyrazino[1,2-b]indazole-3-carboxylate (18) (2.5 g, 4.42 mmol) in CH₃CN (20 mL) and H₂O (20 mL) was added LiOH·H₂O (185.45 mg, 4.42 mmol) and the reaction mixture was stirred at 20° C. for 1 h. The mixture was monitored by LCMS. The mixture was adjusted to pH=4 with HCl (1N) and extracted with 5% EtOH in DCM(30 mL×3). The combined organic layer was washed with brine (30 mL), dried with Na₂SO₄ and concentrated under reduced pressure to give a residue. The residue was purified by prep-HPLC (column: Welch Xtimate C18 250*70 mm #10 um; mobile phase: [water(NH₄HCO₃)-ACN]; B %: 42%-72%, 20 min) to give (R)-10-((8-butoxy-8-oxooctyl)oxy)-6-(tert-butyl)-2-oxo-6,7-dihydro-2H-pyrido[2′,1′:3,4]pyrazino[1,2-b]indazole-3-carboxylic acid (Compound I.10) (1.80 g, 96.1% purity) as a yellow solid.

¹H NMR (400 MHz, DMSO-d₆) δ ppm 8.95; (s, 1 H), 7.63; (d, J=8.4 Hz, 1 H), 7.33-7.20; (m, 2 H), 6.82; (d, J=7.6 Hz, 1 H), 5.26-5.05; (m, 2 H), 4.96; (d, J=4.4 Hz, 1 H), 4.22-4.10; (m, 2 H), 4.00; (t, J=6.4 Hz, 2 H), 2.29; (t, J=7.2 Hz, 2 H), 1.81; (quin, J=6.8 Hz, 2 H), 1.59-1.42; (m, 6 H), 1.40-1.25; (m, 6 H), 0.87; (t, J=7.6 Hz, 3 H), 0.71; (s, 9 H). Mass-Spectrum calculated: 551.3, found: [M+H]⁺=552.3.

### Compound I.11

### Synthetic Route:

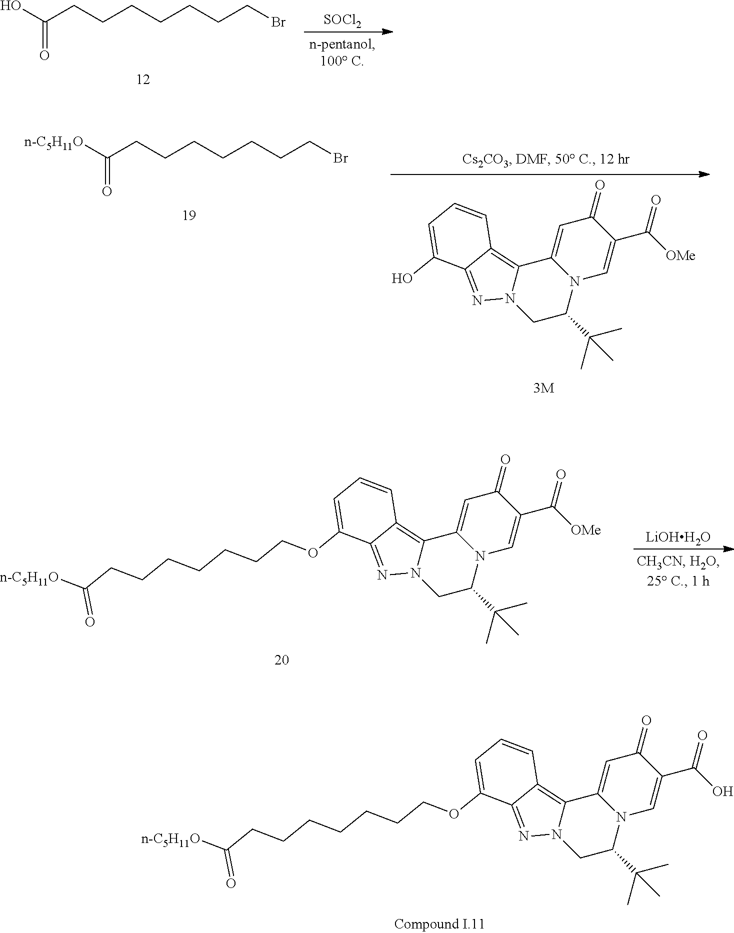

### Procedure for Preparation of pentyl 8-bromooctanoate (19)

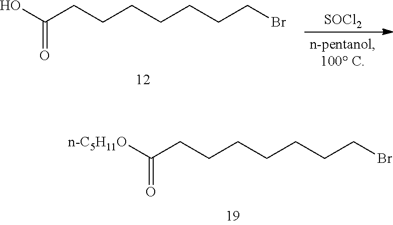

To a mixture of 8-bromooctanoic acid (12) (3.00 g, 13.45 mmol, 1 eq.) in pentan-1-ol (30 mL) was dropwise added SOCl₂ (3.20 g, 26.89 mmol, 1.95 mL, 2 eq.) at 0° C. The mixture was stirred at 100° C. for 1 hour. TLC (Petroleum ether:Ethyl acetate=5:1, R_{f}=0.5) showed reaction was completed. The mixture was concentrated in vacuum and poured into saturated NaHCO₃ (30 mL). The aqueous phase was extracted with ethyl acetate (30 mL×3). The organic phase was washed with brine (20 mL×2), dried with Na₂SO₄, filtered and concentrated in vacuum. The residue was purified by silica gel chromatography (SiO₂, Petroleum ether:Ethyl acetate=1:0 to 5:1) to give pentyl 8-bromooctanoate (3.7 g, 12.62 mmol, 94% yield) as a colorless oil.

¹H NMR (400 MHz, CDCl₃) δ ppm 4.07; (t, J=6.8 Hz, 2 H), 3.41; (t, J=6.8 Hz, 2 H), 2.30; (t, J=7.2 Hz, 2 H), 1.89-1.84; (m, 2 H), 1.66-1.60; (m, 4 H), 1.48-1.42; (m, 2 H), 1.36-1.33; (m, 8 H), 0.93-0.90; (m, 3 H).

### Procedure for Preparation of Methyl (R)-6-(tert-butyl)-2-oxo-10-((8-oxo-8-(pentyloxy)octyl)oxy)-6,7-dihydro-2H-pyrido[2′,1′:3,4]pyrazino[1,2-b]indazole-3-carboxylate (20)

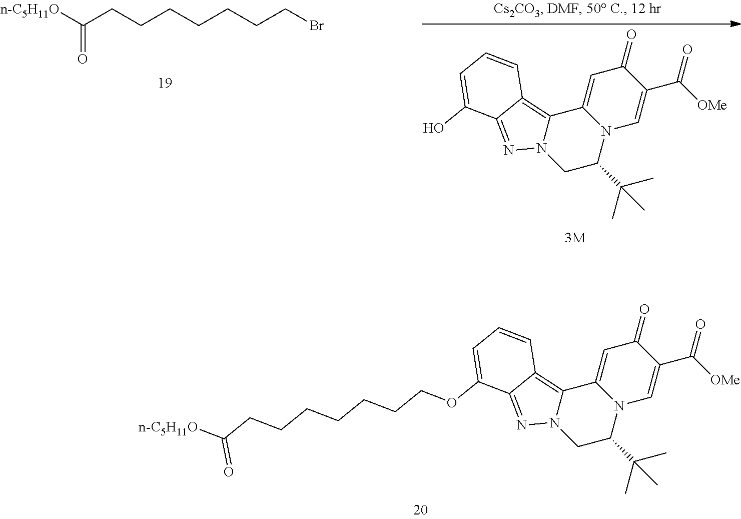

To a mixture of pentyl 8-bromooctanoate (19) (2.39 g, 8.17 mmol, 1.2 eq.) and methyl (R)-6-(tert-butyl)-10-hydroxy-2-oxo-6,7-dihydro-2H-pyrido[2′,1′:3,4]pyrazino[1,2-b]indazole-3-carboxylate (3M) (2.5 g, 6.80 mmol, 1 eq.) in DMF (30 mL) was added Cs₂CO₃ (7.76 g, 23.82 mmol, 3.5 eq.) in one portion at 25° C. The mixture was stirred at 50° C. for 12 h. LCMS showed the reaction was complete. The combine mixture was poured into water (50 mL). The aqueous phase was extracted with ethyl acetate (30 mL×3). The combined organic phase was washed with brine (40 mL×2), dried with Na₂SO₄, filtered and concentrated under reduced pressure. The residue was purified by silica gel chromatography (SiO₂, Ethyl acetate:MeCN=1:0 to 0:1) to give methyl (R)-6-(tert-butyl)-2-oxo-10-((8-oxo-8-(pentyloxy)octypoxy)-6,7-dihydro-2H-pyrido[2′,1′:3,4]pyrazino[1,2-b]indazole-3-carboxylate (20) (2.40 g, 3.93 mmol, 58% yield) as a yellow solid.

¹H NMR (400 MHz, DMSO-d₆) δ ppm 8.57; (s, 1 H), 7.51; (d, J=8.4 Hz, 1 H), 7.18; (t, J=8.0 Hz, 1 H), 6.82; (s, 1 H), 6.78; (d, J=7.6 Hz, 1 H), 5.14-4.98; (m, 2 H), 4.71; (d, J=4.4 Hz, 1 H), 4.17-4.09; (m, 2 H), 3.99; (t, J=6.4 Hz, 2 H), 3.77; (s, 3 H), 2.29; (t, J=7.2 Hz, 2 H), 1.84-1.78; (m, 2 H), 1.59-1.43; (m, 6 H), 1.38-1.25; (m, 8 H), 0.89-0.80; (m, 3 H), 0.70; (s, 9 H).

### Procedure for Preparation of (R)-6-(tert-butyl)-2-oxo-10-((8-oxo-8-(pentyloxy)octyl)oxy)-6,7-dihydro-2H-pyrido[2′,1′:3,4]pyrazino[1,2-b]indazole-3- carboxylic (Compound I.11)

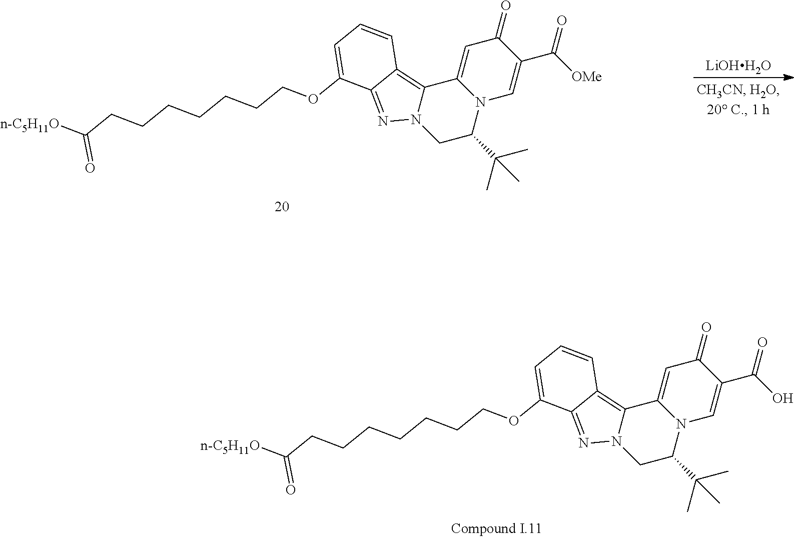

To a mixture of methyl (R)-6-(tert-butyl)-2-oxo-10-((8-oxo-8-(pentyloxy)octypoxy)-6,7-dihydro-2H-pyrido[2′,1′:3,4]pyrazino[1,2-b]indazole-3-carboxylate (20) (2.4 g, 4.14 mmol, 1 eq.) in CH₃CN (14 mL) and H₂O (14 mL) was added LiOH·H₂O (261 mg, 6.21 mmol, 1.5 eq.) in one portion at 25° C. The mixture was stirred at 25° C. for 1 hour. LCMS showed the reaction was completed. The mixture was adjusted pH=6-7 by 1M HCl. The mixture was extracted with ethyl acetate (30 mL×3). The organic phase was washed with brine (10 mL×2), dried with Na₂SO₄, filtered and concentrated in vacuum. The residue was purified by prep-HPLC (column: Agela DuraShell C18 250*70 mm*10 um; mobile phase: [water (NH₄HCO ₃)-ACNI]; B %: 45%-80%, 20 min) to give of (R)-6-(tert-butyl)-2-oxo-10-((8-oxo-8-(pentyloxy)octypoxy)-6,7-dihydro-2H-pyrido[2′,1′:3,4]pyrazino[1,2-b]indazole-3-carboxylic acid (Compound I.11) (1.38 g, 2.37 mmol, 57% yield) as a yellow solid.

¹H NMR (400 MHz, DMSO-d₆) δ ppm 8.95; (s, 1 H), 7.62; (d, J=8.0 Hz, 1 H), 7.33-7.14; (m, 2 H), 6.81; (d, J=7.2 Hz, 1 H), 5.25-5.03; (m, 2 H), 4.96; (s, 1 H), 4.15; (s, 2 H), 3.99; (t, J=6.4 Hz, 2 H), 2.29; (t, J=7.2 Hz, 2 H), 1.86-1.74; (m, 2 H), 1.60-1.42; (m, 6 H), 1.39-1.23; (m, 8 H), 0.87-0.80; (m, 3 H), 0.70; (s, 9 H).

### Compound I.12

### Synthetic Route:

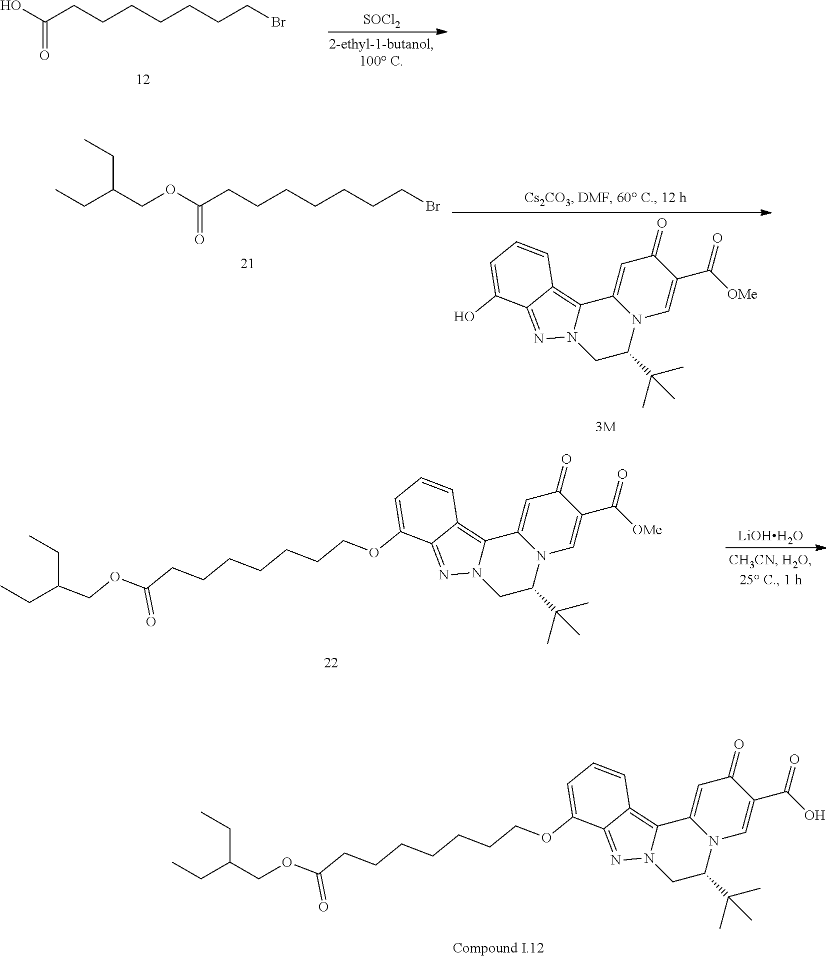

### Procedure for Preparation of 2-ethylbutyl 8-bromooctanoate (21)

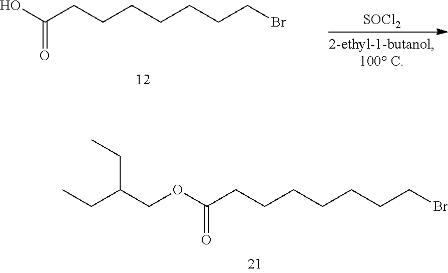 [SMILES: O=C(O)CCCCCCCBr ; "SOCl2" ; CCC(CC)COC(=O)CCCCCCCBr]

To a solution of 8-bromooctanoic acid (12) (3 g, 13.45 mmol, 1 eq) in 2-ethylbutan-1-ol (20 mL) was added SOCl₂ (3.20 g, 26.89 mmol, 1.95 mL, 2 eq). The mixture was stirred at 100° C. for 2 h. TLC

(Petroleum ether:Ethyl acetate=5:1, R_{f}=0.80) indicated the reaction was completed. The reaction mixture was diluted with H₂O (30 mL) and extracted with EtOAc (20 mL×3). The combined organic layers were washed with brine (30 mL), dried over Na₂SO₄, filtered and concentrated under reduced pressure. The residue was purified by column chromatography (SiO₂, Petroleum ether:Ethyl acetate=100:1 to 20:1) to give 2-ethylbutyl 8-bromooctanoate (21) (4.1 g, 13.34 mmol, 99% yield) as colorless oil.

¹H NMR (400 MHz, CDCl₃) δ ppm 4.04-3.98; (m, 2 H), 3.48-3.33; (m, 2 H), 2.40-2.24; (m, 2 H), 1.93-1.80; (m, 2 H), 1.63; (d, J=1.2 Hz, 2 H), 1.55-1.46; (m, 2 H), 1.40-1.33; (m, 9 H), 0.91-0.88; (m, 6 H).

### Procedure for Preparation of methyl (R)-6-(tert-butyl)-10-((8-(2-ethylbutoxy)-8-oxooctyl)oxy)-2-oxo-6,7-dihydro-2H-pyrido[2′,1′:3,4]pyrazino[1,2-b]indazole-3-carboxylate (22)

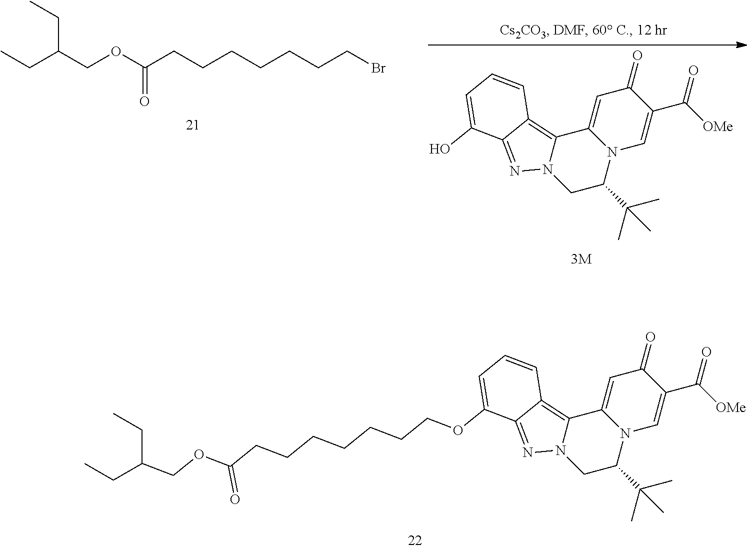 [SMILES: CCC(CC)COC(=O)CCCCCCCBr ; COC(=O)c1cn2c(cc1=O)-c1c3cccc(O)c3nn1C[C@H]2C(C)(C)C ; CCC(CC)COC(=O)CCCCCCCOc1cccc2c3n(nc12)C[C@@H](C(C)(C)C)n1cc(C(=O)OC)c(=O)cc1-3]

To a solution of 2-ethylbutyl 8-bromooctanoate (21) (2.51 g, 8.17 mmol, 1.2 eq.) and methyl (R)-6-(tert-butyl)-10-hydroxy-2-oxo-6,7-dihydro-2H-pyrido[2′,1′:3,4]pyrazino[1,2-b]indazole-3-carboxylate (3M) (2.5 g, 6.8 mmol, 1 eq.) in DMF (30 mL) was added Cs₂CO₃ (7.76 g, 23.82 mmol, 3.5 eq.) at 25° C. The mixture was stirred at 60° C. for 12 hrs. LCMS showed the reaction was completed. The reaction mixture was diluted with H₂O (100 mL) and extracted with EtOAc (50 mL×3). The combined organic layers were washed with brine (50 mL×3), dried over Na₂SO₄, filtered and concentrated under reduced pressure. The residue was purified by column chromatography (SiO₂, Petroleum ether:Ethyl acetate=20:1 to 0:1) to give methyl (R)-6-(tert-butyl)-10-((8-(2-ethylbutoxy)-8-oxooctyl)oxy)-2-oxo-6,7-dihydro-2H-pyrido[2′,1′:3,4]pyrazino[1,2-b]indazole-3-carboxylate (22) (2.9 g, 4.88 mmol, 71.7% yield) as yellow solid.

¹H NMR (400 MHz, CDCl₃) δ ppm 8.28; (s, 1 H), 7.39; (d, J=8.4 Hz, 1 H), 7.17; (t, J=8.0 Hz, 1 H), 7.02; (s, 1 H), 6.65; (d, J=7.2 Hz, 1 H), 5.15; (d, J=14.8 Hz, 1 H), 4.92; (dd, J=5.4, 14.8 Hz, 1 H), 4.18; (t, J=6.4 Hz, 2 H), 4.10; (d, J=5.2 Hz, 1 H), 3.99; (d, J=6.0 Hz, 2 H), 3.93; (s, 3 H), 2.31; (t, J=7.6 Hz, 2 H), 2.02-1.92; (m, 2 H), 1.64; (quin, J=7.2 Hz, 2 H), 1.56-1.47; (m, 3 H), 1.43-1.31; (m, 8 H), 0.89; (t, J=7.6 Hz, 6 H), 0.83; (s, 9 H).

### Procedure for Preparation of (R)-6-(tert-butyl)-104(8-(2-ethylbutoxy)-8-oxooctyl)oxy)-2-oxo-6,7-dihydro-2H-pyrido[2′,1′:3,4]pyrazino[1,2-b]indazole-3-carboxylic (Compound I.12)

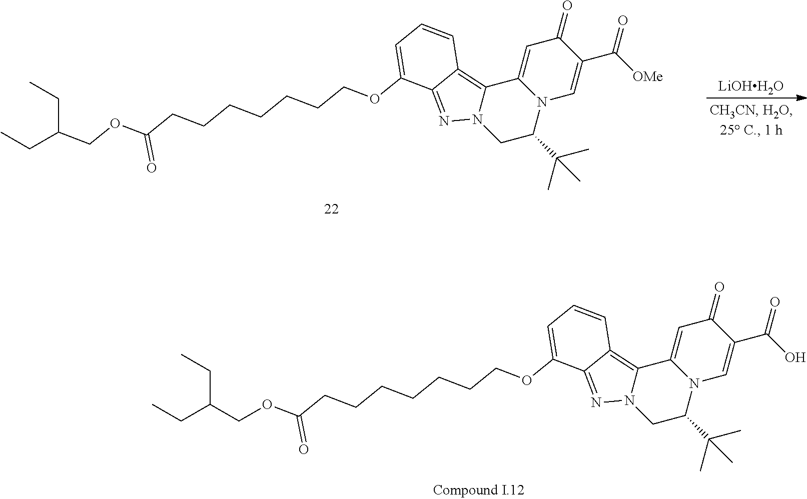 [SMILES: CCC(CC)COC(=O)CCCCCCCOc1cccc2c3n(nc12)C[C@@H](C(C)(C)C)n1cc(C(=O)OC)c(=O)cc1-3 ; "LiOH·H2O" ; CCC(CC)COC(=O)CCCCCCCOc1cccc2c3n(nc12)C[C@@H](C(C)(C)C)n1cc(C(=O)O)c(=O)cc1-3]

To a solution of methyl (R)-6-(tert-butyl)-10-((8-(2-ethylbutoxy)-8-oxooctyl)oxy)-2-oxo-6,7-dihydro-2H-pyrido[2′,1′:3,4]pyrazino[1,2-b]indazole-3- carboxylate (22) (2.9 g, 4.88 mmol, 1 eq.) in acetonitrile (15 mL) and H₂O (15 mL) was added LiOH·H₂O (266.45 mg, 6.35 mmol, 1.3 eq.) at 25° C. The mixture was stirred at 25° C. for 1 h. LCMS showed the reaction was complete. The reaction mixture was diluted with H₂O (20 mL) and adjusted to pH=7 by diluted HCl (1 M), extracted with EtOAc (20 mL×3). The combined organic layers were washed with brine (30 mL), dried over Na₂SO₄, filtered and concentrated under reduced pressure. The residue was purified by prep-HPLC (column: Agela DuraShell C18 250*70 mm*10 um; mobile phase: [water(NH₄HCO₃)-ACN]; B %: 45%-80%, 20 min) to give (R)-6-(tert-butyl)-10-((8-(2-ethylbutoxy)-8-oxooctyl)oxy)-2-oxo-6,7-dihydro-2H-pyrido[2′,1′:3,4]pyrazino[1,2-b]indazole-3-carboxylic (Compound I.12) (1.15 g, 1.98 mol, 41% yield) as a yellow solid.

¹H NMR (400 MHz, DMSO-d₆) δ ppm 8.95; (s, 1 H), 7.63; (d, J=8.0 Hz, 1 H), 7.29-7.18; (m, 2 H), 6.82; (d, J=7.6 Hz, 1 H), 5.24-5.06; (m, 2 H), 4.96; (s, 1 H), 4.16; (s, 2 H), 3.93; (d, J=5.6 Hz, 2 H), 2.30; (d, J=7.2 Hz, 2 H), 1.89-1.74; (m, 2 H), 1.58-1.51; (m, 2 H), 1.50-1.41; (m, 3 H), 1.38-1.24; (m, 8 H), 0.83; (t, J=7.2 Hz, 6 H), 0.71; (s, 9 H).

### Compound I.13

### Route:

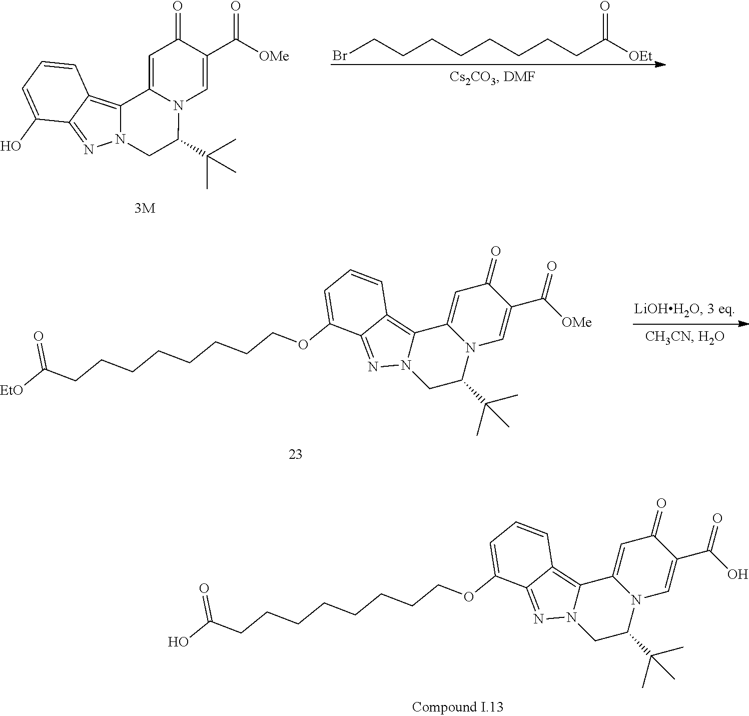 [SMILES: COC(=O)c1cn2c(cc1=O)-c1c3cccc(O)c3nn1C[C@H]2C(C)(C)C ; CCOC(=O)CCCCCCCCBr ; CCOC(=O)CCCCCCCCOc1cccc2c3n(nc12)C[C@@H](C(C)(C)C)n1cc(C(=O)OC)c(=O)cc1-3 ; CC(C)(C)[C@@H]1Cn2nc3c(OCCCCCCCCC(=O)O)cccc3c2-c2cc(=O)c(C(=O)O)cn21]

### Procedure for Preparation of ethyl (R)-6-(tert-butyl)-104(9-ethoxy-9-oxononyl)oxy)-2-oxo-6,7-dihydro-2H-pyrido[2′,1′:3,4]pyrazino[1,2-b]indazole-3- carboxylate (23)

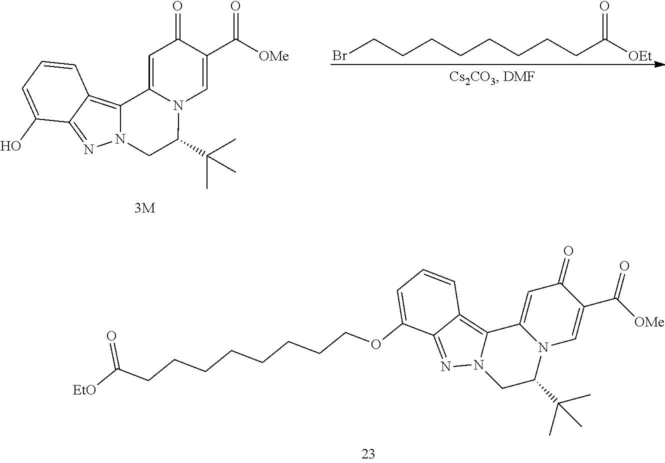 [SMILES: COC(=O)c1cn2c(cc1=O)-c1c3cccc(O)c3nn1C[C@H]2C(C)(C)C ; CCOC(=O)CCCCCCCCBr ; CCOC(=O)CCCCCCCCOc1cccc2c3n(nc12)C[C@@H](C(C)(C)C)n1cc(C(=O)OC)c(=O)cc1-3]

To a solution of ethyl (R)-6-(tert-butyl)-10-hydroxy-2-oxo-6,7-dihydro-2H-pyrido[2′,1′:3,4]pyrazino[1,2-b]indazole-3-carboxylate (3M) (1 g, 2.62 mmol, 1 eq) in DMF (10 mL) was added Cs₂CO₃ (2.56 g, 7.86 mmol, 3 eq) and ethyl 9-bromononanoate (903.22 mg, 3.41 mmol, 1.3 eq), stirred at 60° C. for 4 h. The mixture was diluted with H₂O (50 mL), extracted ethyl acetate (50 mL×3). The combined organic phase was washed with brine (100 mL×2), dried with anhydrous Na₂SO₄, filtered and concentrated in vacuum. The residue was purified by column chromatography (SiO₂, Petroleum ether:Ethyl acetate=1:0 to 0:1) to give ethyl (R)-6-(tert-butyl)-10-hydroxy-2-oxo-6,7-dihydro-2H-pyrido[2′,1′:3,4]pyrazino[1,2-b]indazole-3-carboxylate (23) (600 mg, yield: 40%) was a yellow solid.

Mass-Spectrum calc.: 565.71, found: [M+H]⁺+=566.4.

¹H NMR (400 MHz, DMSO-d₆) δ ppm 8.53; (s, 1 H), 7.50; (d, J=8.4 Hz, 1 H), 7.18; (t, J=8.0 Hz, 1 H), 6.87-6.76; (m, 2 H), 5.15-4.97; (m, 2 H), 4.70; (d, J=4.4 Hz, 1 H), 4.24; (q,J=7.2 Hz, 2 H), 4.14; (dt, J=2.8, 6.4 Hz, 2H), 4.04; (q, J=7.2 Hz, 2 H), 2.26; (t, J=7.2 Hz, 2 H), 1.80; (quin, J=6.8 Hz, 2 H), 1.58-1.41; (m, 4 H), 1.39-1.24; (m, 9 H), 1.16; (t, J=7.2 Hz, 3 H), 0.70; (s, 9 H).

### Procedure for Preparation of (R)-6-(tert-butyl)-10-((8-carboxyoctyl)oxy)-2-oxo-6,7-dihydro-2H-pyrido[2′,1′:3,4]pyrazino[1,2-b]indazole-3-carboxylic Acid (Compound I.13)

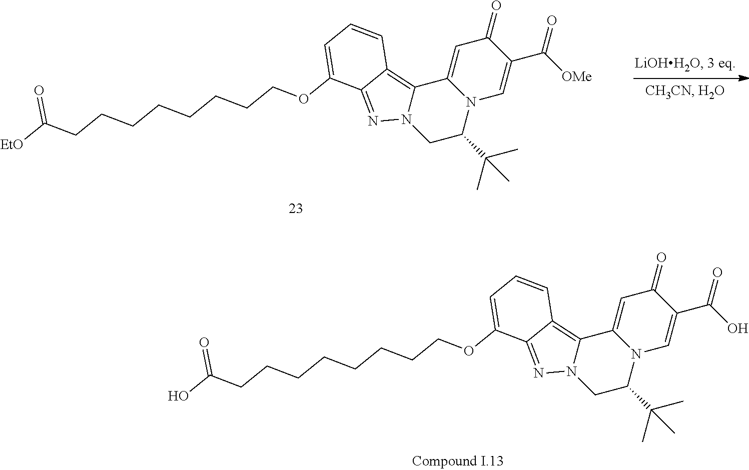 [SMILES: CCOC(=O)CCCCCCCCOc1cccc2c3n(nc12)C[C@@H](C(C)(C)C)n1cc(C(=O)OC)c(=O)cc1-3 ; CC(C)(C)[C@@H]1Cn2nc3c(OCCCCCCCCC(=O)O)cccc3c2-c2cc(=O)c(C(=O)O)cn21]

To a mixture of methyl (R)-6-(tert-butyl)-10-((9-ethoxy-9-oxononyl)oxy)-2-oxo-6,7-dihydro-2H-pyrido[2′,1′:3,4]pyrazino[1,2-b]indazole-3-carboxylate (23) (600 mg, 1.06 mmol, 1 eq) in CH₃CN (6 mL) and H₂O (6 mL) was added LiOH·H₂O (133.51 mg, 3.18 mmol, 3 eq), stirred at 25° C. for 2 h. The pH of the reaction solution was adjusted to 7 with 1M HCl solution at 0° C. The reaction solution was purified by prep-HPLC (column: Waters Xbridge BEH C18 250*70 mm*10 um; mobile phase: [water(NH₄HCO₃)-ACN]; B %: 10%-40%, 20 min) to give (R)-6-(tert-butyl)-10-((8-carboxyoctyl)oxy)-2-oxo-6,7-dihydro-2H-pyrido[2′,1′:3,4]pyrazino[1,2-b]indazole-3- carboxylic acid (Compound I.13) (400 mg, yield: 71%, purity: 96.35%) as a green solid.

¹H NMR (400 MHz, DMSO-d₆) δ ppm 8.94; (s, 1 H), 7.62; (br d, J=8.4 Hz, 1 H), 7.33-7.15; (m, 2 H), 6.82; (d, J=7.6 Hz, 1 H), 5.24-5.08; (m, 2 H), 4.96; (br d, J=4.4 Hz, 1 H), 4.23-4.11; (m, 2 H), 2.18; (t, J=7.2 Hz, 2 H), 1.88-1.76; (m, 2 H), 1.57-1.42; (m, 4 H), 1.40-1.23; (m, 6 H), 0.71; (s, 9 H). Mass-Spectrum calculated: 509.6, found: [M+H]⁺=510.0.

### Compound I.14

### Procedure for Preparation of (R)-6-(tert-butyl)-10-((9-ethoxy-9-oxononyl)oxy)-2-oxo-6,7-dihydro-2H-pyrido[2′,1′:3,4]pyrazino[1,2-b]indazole-3- carboxylic Acid (Compound I.14)

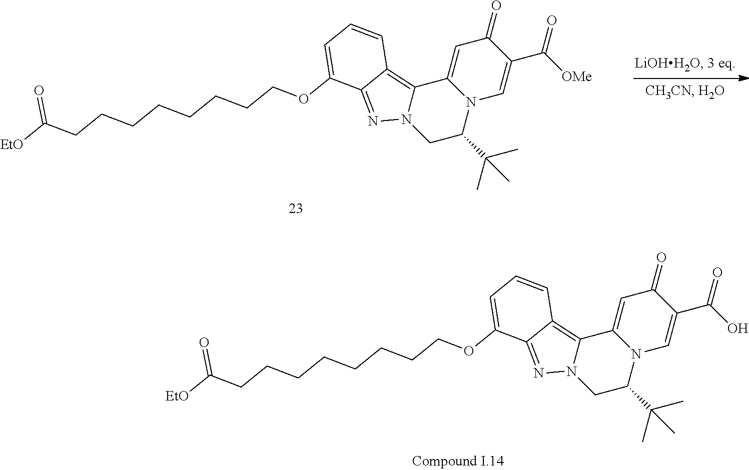 [SMILES: CCOC(=O)CCCCCCCCOc1cccc2c3n(nc12)C[C@@H](C(C)(C)C)n1cc(C(=O)OC)c(=O)cc1-3 ; CCOC(=O)CCCCCCCCOc1cccc2c3n(nc12)C[C@@H](C(C)(C)C)n1cc(C(=O)O)c(=O)cc1-3]

A mixture of methyl (R)-6-(tert-butyl)-10-((9-ethoxy-9-oxononyl)oxy)-2-oxo-6,7-dihydro-2H-pyrido[2′,1′:3,4]pyrazino[1,2-b]indazole-3-carboxylate (23) (480 mg, 870 umol, 1 eq) and LiOH·H₂O (36 mg, 870.08 umol, 1 eq) in MeCN (4.8 mL) and H₂O (4.8 mL) was stirred at 25° C. for 1 hr. The pH of the reaction solution was adjusted to 7 with 1M HCl solution at 0° C. The mixture was concentrated under reduced pressure. The residue was purified by prep-HPLC (column: Waters Xbridge BEH C18 250*70 mm*10 um; mobile phase: [water(NH₄HCO₃)-ACN]; B %: 10%-40%, 20 min) to give (R)-6-(tert-butyl)-10-((9-ethoxy-9-oxononyl)oxy)-2-oxo-6,7-dihydro-2H-pyrido[2′,1′:3,4]pyrazino[1,2-b]indazole-3-carboxylic acid (Compound I.14) (146.5 mg, yield: 31%, purity: 98.77%) as a green solid.

¹H NMR (400 MHz, DMSO-d₆) δ ppm 8.95; (s, 1 H), 7.63; (d, J=8.0 Hz, 1 H), 7.32-7.17; (m, 2 H), 6.82; (d, J=8.0 Hz, 1 H), 5.22-5.06; (m, 2 H), 4.96; (s, 1 H), 4.15; (s, 2H), 4.07-3.97; (m, 2 H), 2.27; (t, J=6.8 Hz, 2H), 1.90-1.72; (m, 2 H), 1.57-1.41; (m, 4 H), 1.40-1.24; (m, 6 H), 1.20-1.11; (m, 3 H), 0.70; (s, 9 H).

Mass-Spectrum calculated: 537.66, found: [M+H]⁺=538.1.

While Compound (1) was prepared as described using methods disclosed in published PCT applications WO 2018/198079 and U.S. Pat. No. 10,301,312 B2 Date of Patent: May 28, 2019 whose contents are incorporated entirely by this reference, we include the general procedure below for clarity. From commercially available (24), Compound (25) was obtained by alkylation with potassium carbonate in acetonitrile. The nitro group of Compound (25) was reduced using hydrogen over the catalyst of palladium on carbon to afford Compound (26). Compound (26) was treated with isoamyl nitrite and acetic anhydride to afford the indazole(27). Alkylation of (27) was effected by treatment with (28) in the presence of lithium carbonate with warming in dioxane to afford (29) as the major regioisomer (minor regioisomer separated by chromatography). Compound (29) was formylated using N-formylmorpholine to afford (30). Deprotection and concomitant cyclization was accomplished by treatment with trifluoroacetic acid to afford Compound (31). Compound (31) was condensed with Compound (32) to afford the tetracycle (33) in quantitative yield. Desaturation of Compound (33) was effected by treatment with DDQ to afford Compound (1).

### Synthesis of Intermediate 1

### General Scheme:

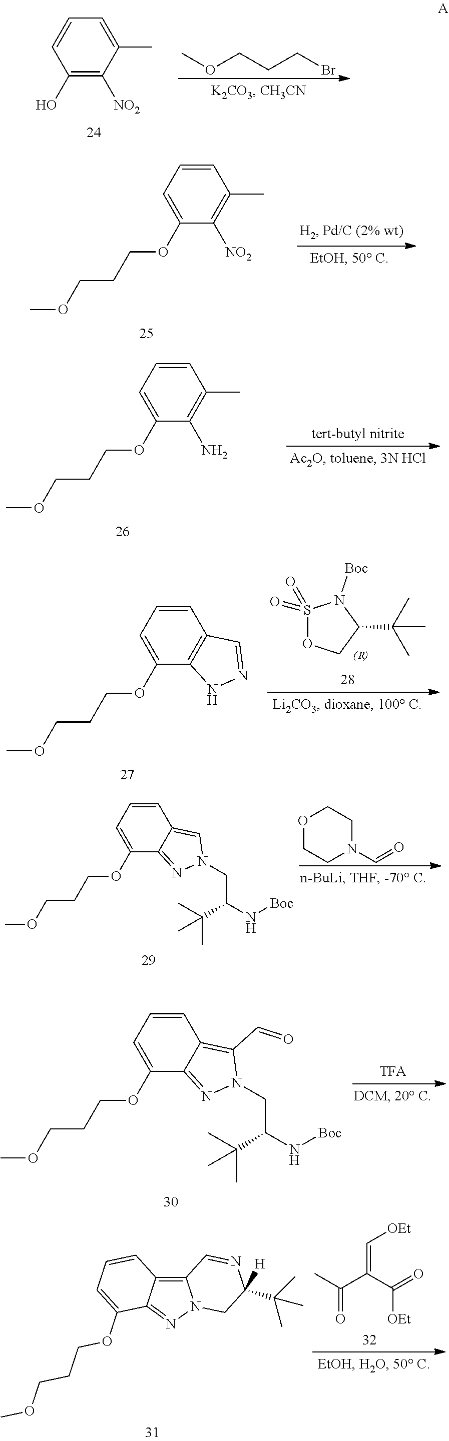

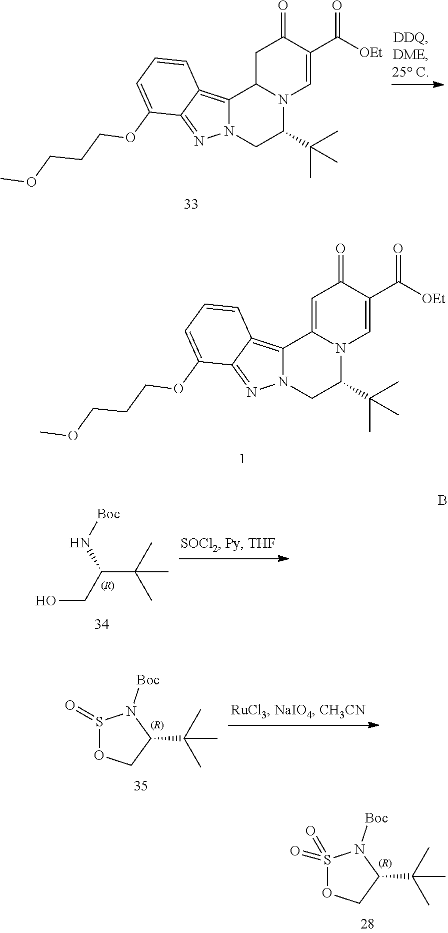

### Preparation of 1-(3-methoxypropoxy)-3-methyl-2-nitrobenzene (25)

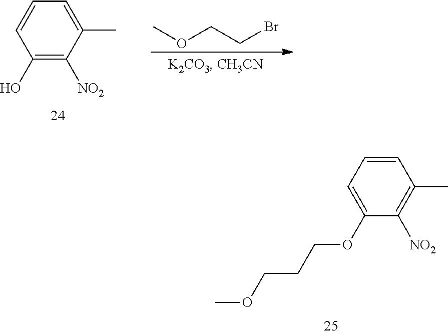

Acetonitrile (5 v) was charged to a 100 L reactor, then 24 (10.0 kg, 65.3 mol, 1 eq) was added at 25° C. K₂CO₃ (10.8 kg, 78.4 mol, 1.2 eq) was added in one portion at 22° C., the color changed from yellow to red. 1-Bromo-3-methoxypropane (11.0 kg, 71.8 mol, 1.1 eq) was added dropwise at 25° C. over 5 min while the reaction temperature did not change. The resulting mixture was heated to 70° C. and stirred for 16 h under nitrogen. The mixture was then cooled to 25° C., filtered and the solid was washed by methyl tert-butyl ether (MTBE, 2 v). The filtrate was concentrated in vacuo to give crude product as an oil. The oil was dissolved in MTBE (2 v), then washed with 2N NaOH solution (0.3 eq), the aqueous phase was extracted with MTBE (1 v×2). The combined organic phase was washed with brine (1V), dried over anhydrous Na₂SO₄, filtered and concentrated in vacuo to give 25 (13.9 kg, 94.5% yield) as a yellow oil.

### Preparation of 2-(3-methoxypropoxy)-6-methylaniline (26)

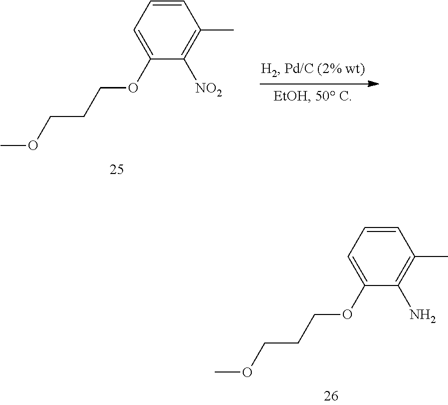

Ethanol (10 v) was charged to a 200 L reactor, then 25 (13.9 kg, 61.71 mol, 1 eq) was added at 25° C. Palladium on carbon (278 g, 2% wt) was added in one portion at 25° C. The suspension was degassed under vacuum and purged with H₂ three times. The resulting mixture was heated to 50° C. and stirred for 48 h under hydrogen. The mixture was cooled to 25° C., filtered and the solid was washed by ethanol (2 v). The filtrate was concentrated in vacuo to give 26 (11.5 kg, 93.8% yield) as a brown oil.

### Preparation of 7-(3-methoxypropoxy)-1H-indazole (27)

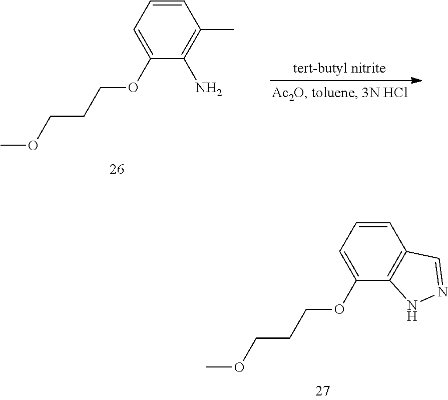

Toluene (10 v) was charged to a 250 L reactor, then 26 (9.0 kg, 46.09 mol, 1 eq) was added at 25° C. Potassium acetate (5.4 kg, 55.31 mol, 1.2 eq) was added in one portion at 25° C. Acetic anhydride (14.1 kg, 138.28 mol, 12.95 L, 3 eq) was added dropwise at 25° C. The mixture was heated to 50° C. and stirred for 1 h under N₂. Tert-butyl nitrite (11.9 kg, 115.23 mol, 13.7 L, 2.5 eq) was added dropwise at 50° C. The resulting mixture was heated between 60 and 65° C. and stirred for 16 h under nitrogen. The mixture was cooled to 25° C., quenched by water (3 V). The product was extracted with ethyl acetate (2 V×2). The combined organic phase was washed with brine (2 V), dried with anhydrous Na₂SO₄, filtered and concentrated in vacuo at 40° C. to afford a crude product. The crude product was dissolved in MeOH (4 V), then 3N HCl solution (3 V) was added dropwise while maintaining the reaction temperature no higher than 35° C. and then stirred for 1 h at 45° C. The mixture was concentrated in vacuo to move MeOH at 40° C. and the aqueous phase was extracted with ethyl acetate (2 V×3). The combined organic phase was washed with brine (2 V), dried with anhydrous Na₂SO₄, filtered and concentrated in vacuo at 40° C. to afford crude 27 as yellow oil. The crude product was purified by silica gel column chromatography (n-heptane/ethyl acetate, 10:1 to 5:1) to afford crude 27 as yellow oil. The oil was dissolved in MTBE (1.5 v) and stirred for 1 h, yellow solid precipitated. The mixture was filtered and the filter cake was washed by n-heptane/MTBE (1.6 V:0.4 V) two times and dried in vacuo to afford 27 (7.0 kg, yield: 73%).

### Preparation of tert-butyl (R)-(1-(7-(3-methoxypropoxy)-2H-indazol-2-yl)-3,3-dimethylbutan-2-yl)carbamate (29)

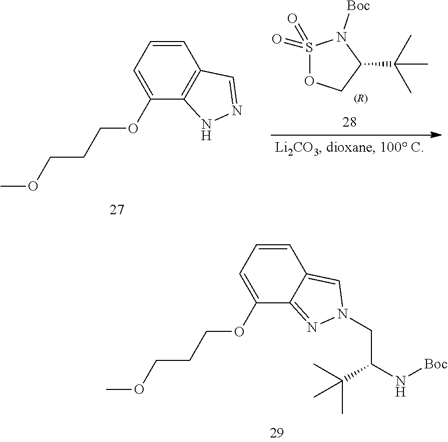

Dioxane (10 V) was charged to a 50 L reactor, followed by 27 (2.8 kg, 13.58 mol, 1 eq) and 28 (5.7 kg, 20.36 mol, 1.5 eq) were added at 25° C. Lithium carbonate (2.0 kg, 27.15 mol, 2 eq) was added in one portion at 25° C. The resulting mixture was heated to 100° C. and stirred for 90 h under N₂. The reaction mixture was filtered and the filter cake was washed with ethyl acetate (0.5 v×2). The filtrate was concentrated under reduced pressure to removed most of dioxane to afford a crude product. The crude product was then triturated with NaOH (1M, 4 v) at 25° C. for 16 h. The reaction mixture was filtered to give a crude product (˜5.5 kg, crude). The crude product was triturated with NaOH (1 M, 4 v) at 25° C. for 16 h. The reaction mixture was filtered to give a crude product (˜5.1 kg, crude). The crude product was triturated with MTBE/n-heptane (2 v:1 v) at 25° C. for 1 hour. The reaction mixture was filtered to give 29 (3.8 kg, 69.0%).

### Preparation of tert-butyl (R)-(1-(3-formyl-7-(3-methoxypropoxy)-2H-indazol-2-yl)-3,3-dimethylbutan-2-yl)carbamate (30)

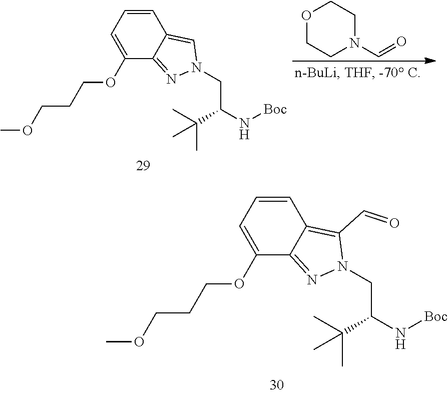 [SMILES: COCCCOc1cccc2cn(C[C@H](NC(=O)OC(C)(C)C)C(C)(C)C)nc12 ; O=CN1CCOCC1 ; COCCCOc1cccc2c(C=O)n(C[C@H](NC(=O)OC(C)(C)C)C(C)(C)C)nc12]

The following procedure was carried out as nine parallel batches. In each batch, tetrahydrofuran (10 v) was charged to a 3 L glass flask, then 29 (540 g, 1.33 mol, 1 eq) was added at 25° C. The mixture was degassed and purged with nitrogen for three times. The mixture was cooled to −60° C. with ethanol and dry ice bath. n-BuLi (2.5 M, 1.86 L, 3.5 eq) was added dropwise at −60° C. under nitrogen for 1.5 h. The mixture was stirred for 0.5 h at −60° C. under nitrogen. N-Formylmorpholine (460 g, 3.99 mol, 400 mL, 3 eq) was added dropwise at −60° C. under nitrogen for 1 h. The mixture was stirred for 2 hours at −60° C. under nitrogen. To the reaction mixture was added saturated NH₄Cl (2 v) dropwise slowly at −60° C. under nitrogen. The reaction mixture was warmed to 25° C. At this stage, all nine batches were combined. The product was extracted with ethyl acetate (2 v×3). The combined organic phase was washed with brine (2 V×2), dried with anhydrous Na₂SO₄, filtered and concentrated in vacuo at 50° C. to afford a crude product. The crude product was triturated with heptane (4 v) at 25° C. for 1 h. The mixture was filtered and the filter cake was washed by heptane (2 v) twice and dried in vacuo to afford 30 (4.3 kg, 82.8%).

### Preparation of (R)-3-(tert-butyl)-7-(3-methoxypropoxy)-3,4-dihydropyrazino[1,2-b]indazole (31)

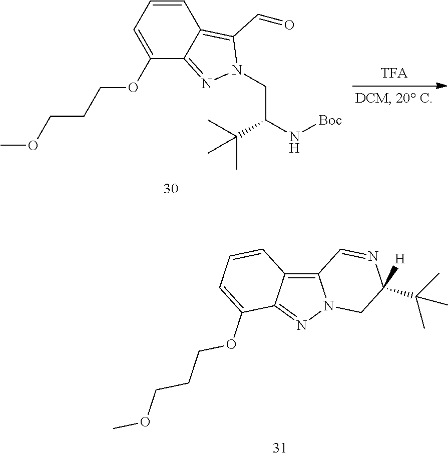 [SMILES: COCCCOc1cccc2c(C=O)n(C[C@H](NC(=O)OC(C)(C)C)C(C)(C)C)nc12 ; COCCCOc1cccc2c3n(nc12)C[C@@H](C(C)(C)C)N=C3]

Dichloromethane (10 v) was charged to a 50 L glass flask, then 30 (3.5 kg, 8.07 mol, 1 eq) was added at 20° C. Trifluoroacetic acid (TFA, 2 v, 7 L) was added dropwise at 20° C. The mixture was stirred for 2 h at 20° C. The reaction mixture concentrated in vacuo at 50° C. to remove dichloromethane and most of TFA. The reaction mixture was diluted with dichloromethane (10 v). To the reaction mixture was added saturated NaHCO₃ (˜10 v) slowly and adjusted pH to 7˜8. The product was extracted with dichloromethane (2 v×2). The combined organic phase was concentrated in vacuo at 50° C. to afford 31 (2.5 kg, 98.2%).

### Preparation of ethyl (6R)-6-(tert-butyl)-10-(3-methoxypropoxy)-2-oxo-1,6,7,13c-tetrahydro-2H-pyrido[2,1′3,4]pyrazino[1,2-b]indazole-3-carboxylate (33)

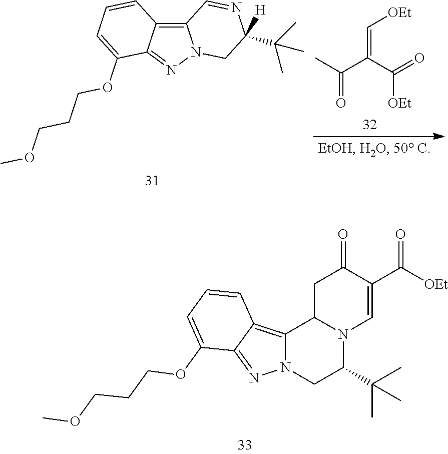

Ethanol (10 v) and water (1 v) were charged to a 50 L reactor. 31 (2.7 kg, 8.56 mol, 1 eq) was added at 25° C. over 30 min. Compound 32 (3.2 kg, 17.12 mol, 2 eq) was added at 25° C. under N₂. After addition, the reaction mixture was elevated to 50° C. The resulting mixture was stirred at 50° C. for 16 h under N₂. The reaction mixture was concentrated to remove the EtOH and H₂O under reduced pressure to afford crude 33 (4.3 kg).

### Preparation of ethyl (R)-6-(tert-butyl)-10-(3-methoxypropoxy)-2-oxo-6,7-dihydro-2H-pyrido[2,1′3,4]pyrazino[1,2-b]indazole-3-carboxylate (1)

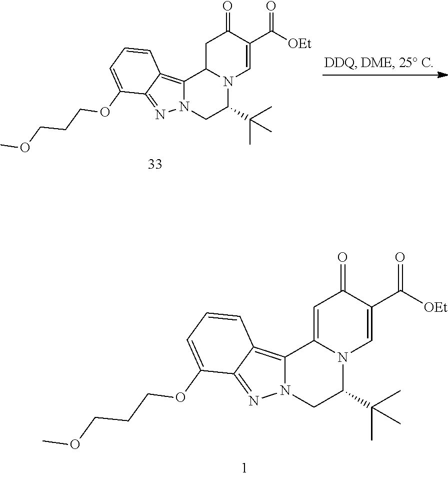 [SMILES: CCOC(=O)C1=CN2C(CC1=O)c1c3cccc(OCCCOC)c3nn1C[C@H]2C(C)(C)C ; CCOC(=O)c1cn2c(cc1=O)-c1c3cccc(OCCCOC)c3nn1C[C@H]2C(C)(C)C]

Dimethoxyethane (DME, 10 V) was charged to a 50 L reactor. 33 (4.3 kg, 9.44 mol, 1 eq) was added at 25° C. over 5 min. 2,3-Dichloro-5,6-dicyano-1,4-benzoquinone (DDQ, 2.57 kg, 11.33 mol, 1.2 eq) was added at 25° C. The mixture was stirred at 25° C. for 2 h. The mixture was concentrated to obtain the residue. Then the residue was dissolved in ethyl acetate (10V). The solution was neutralized with saturated Na₂CO₃ (0.5V), the alkalized reaction mixture was diluted with water (0.6 V), and the organic layer was separated. The water phase was extracted with ethyl acetate (3V×2). The combined organics was washed with water (0.6V), brine (0.6V), and concentrated to obtain the crude product. The crude product was dissolved in ethyl acetate (0.3 V), and then 2N HCl solution in ethyl acetate (9.4 L, 18.88 mol, 2 eq) was added dropwise at 25° C. and stirred for 12 h. The mixture was filtered to obtain yellow solid. The remaining water is removed to give 1 (2.4 kg, 56.0% yield, 95.69% purity by HPLC) as yellow solid.

¹H NMR (400 MHz, DMSO-d₆)

d 8.68; (s, 1H); 7.53; (d, J=8.4 Hz, 1H); 7.24; (t, J=8.0 Hz, 1H); 7.10; (s, 1H); 6.83; (d, J=7.6 Hz, 1H); 5.18-5.09; (m, 2H); 4.81; (d, J=4.8 Hz, 1H); 4.28-4.20; (m, 4H); 3.54; (t, J=6.2 Hz, 2H); 3.27; (s, 3H); 2.07; (quintet, J=6.3 Hz, 2H); 1.30; (t, J=7.0 Hz, 3H) and 0.72; (s, 9H).

### Preparation of tert-butyl (4R)-4-(tert-butyl)-1,2,3-oxathiazolidine-3-carboxylate 2-oxide (35)

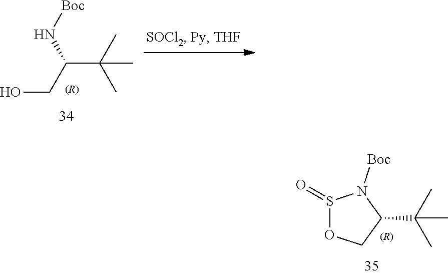

Tetrahydrofuran (1.5 v) was charged to a 50 L glass flask, then pyridine (7.6 kg, 96.64 mol, 7.8 L, 7 eq) was added at 25° C. The reaction mixture was cooled down to 0° C. Thionyl chloride (4.9 kg, 41.42 mol, 3.0 L, 3 eq) was added dropwise over 1 hour at 0 to 10° C. A solution of 34 (3.0 kg, 13.81 mol, 1 eq) in THF (3 v) was added dropwise over 2 hours at 0 to 10° C. The resulting mixture was stirred at 25° C. for 16 h under nitrogen. The reaction mixture was cooled down to 0° C. in the ice bath, and the reaction mixture was added to ice water slowly. The reaction mixture was extracted with EtOAc (2 v×2). The organic phase was separated and washed with brine (1 v×2) two times. The organic was dried over Na₂SO₄ filtered and concentrated under reduced pressure to give a residue (3.6 kg, crude).

### Preparation of tert-butyl (R)-4-(tert-butyl)-1,2,3-oxathiazolidine-3-carboxylate 2,2-dioxide (28)

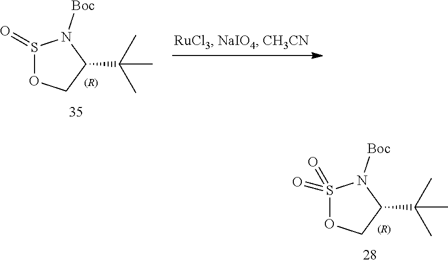

Reaction was carried out in four batches. In each batch, acetonitrile (5 v) and water (5 v) were charged to a 50 L reactor, then 35 (2.5 kg, 9.49 mol, 1 eq) was added at 25° C. over 20 min. The reaction mixture was cooled down to 0° C. over 30 min. RuCl₃ (19.7 g, 94.93 mmol, 6.33 mL, 0.01 eq) was added at 0° C. under nitrogen. NaIO₄ (3.05 kg, 14.24 mol, 789 mL, 1.5 eq) was added in about 10 portions at 0 to 10° C. (solubility of NaIO₄ is 80 g/L at 20° C.) (4 hours). The resulting mixture was stirred at 25° C. for 1.5 h under nitrogen. At this stage, four batches were combined. The mixture was diluted with methyl tert-butyl ether (MTBE, 1.7 v). The reaction mixture was filtered through a pad of Celite. The pad was washed with MTBE (1 v×3). The combined filtrates were extracted with MTBE (1.3 v×3). The combined organic phase was washed with saturated Na₂SO₃ solution (1.7 v×2). The organic phase was separated, washed with brine (1.3 v×2). The organic was dried over Na₂SO₄ filtered and concentrated under reduced pressure to give a crude product. The crude product was triturated with n-heptane/ethyl acetate (2 v/0.4 v) at 25° C. for 30 min. The reaction mixture was filtered and the filter cake was washed with n-heptane (0.33 v). Dried in vacuo to give 28 (7.9 kg, 75%).

### HBsAg Assay

HepG2.2.15 cells were grown in flasks microscopically and seeded into 96-well plates at a concentration of 6.0×10⁴ cells/well and incubated at 37° C., 5% CO₂ overnight.

Source plate preparation. The stock concentration of reference compound and test compounds is 20 mM. Firstly, manually diluted the compound to obtain the first concentration points of 10 μM and 200 μM, respectively. Then a 3-fold, 8 points serial dilution is performed to obtain the 200× source plate.

Source plate compounds were pipetted as follows: 3 μL 200× source plate compounds into sterile 2 mL 96 well plate which contain 297 μL 2% FBS Medium, mix well and transfer 100 μL/well to corresponding cell culture plates. Therefore, the test conditions of the final compounds is 1 μM initiation, 3-fold serial dilution, and 8 concentration points in duplicate.

Incubate the cells at 37° C., 5% CO2 for three days.

24 hours post-seeding, the cells were treated with 200 μl/well of media containing serially diluted compounds in DMSO. DMSO alone was used as the no drug control. The final DMSO concentration in all wells was 0.5%.

The HBsAg kit (Auto Biology-CL 0310) was used to determine the level of secreted HBVsAg. The HBSAg assay was performed as follows:
a) Blance the ELISA kit at room temperature for 30 minutes.
b) Add 50 μL standard solution, sample, positive and negative control to each well of the microplate in the kit.
c) Add 50 μL HBsAg enzyme conjugate to each well.
d) Attach the plate membrane, oscillate for 60 seconds, and incubate at 37° C. for 60 minutes.
e) Add 350 μL washing buffer to each well, shake slightly, discard liquid, repeat 6 times. Then pat 5 times on an absorbent paper towel to dry the hole.
f) Add 50 μL mixture of reagent A and B to each well. Oscillate the microplate for 10 seconds.
g) Cover the plate membrane and incubate at room temperature for 10 minutes. Read the luminescent signal using Biotek-Synergy 2.

### Data Analysis

Compounds inhibition against HBsAg at different concentrations are calculated by the following formula:

Inhibition %=(1−value of sample/ave. value of control)×100

50% effective concentrations (EC₅₀) and were calculated with the GraphPad Prism software.

TABLE 3
Activity of select compounds of Formula (I) in HepG2
HBV S-antigen secretion biological assay (geometrical
mean ± standard deviation, if applicable):

| | | Number of |
|---|---|---|
| Compound | EC₅₀, nM | repeats, n |
| I.1 | 25.5 | 1 |
| I.2 | 6.5 ± 2.5 | 3 |
| I.3 | 11.8 | 1 |
| I.4 | 14.0 | 1 |
| I.6 | 2.6 ± 0.6 | 3 |
| I.7 | 7.1 | 1 |
| I.9 | 5.3 ± 0.9 | 3 |
| I.10 | 4.0 ± 0.8 | 3 |
| I.13 | 1.2 ± 0.5 | 3 |

### Uptake Transporters

The OATP1B1 and OATP1B3 substrate assays were conducted at Wuxi utilizing the human embryonic kidney cell line, HEK293, stably transfected with human transporter genes. The identification of potential transporter substrates are achieved by measuring the Uptake Fold values in the transfected HEK293-OATP1B1 and OATP1B3 cells line and the HEK293-MOCK cell line in the Presence and Absence of positive inhibitors.

The objective of this study was to determine whether Compound I.2 and Compound I.6 are potential OATP1B1 and OATP1B3 uptake substrates.

The detailed information of substrates, positive inhibitors and internal standards are shown in the following table.

| | | | | Catalog |
|---|---|---|---|---|
| Transporter | Application | Compound ID | Source | Number |
| — | Internal | Tolbutamide | Sigma- | T3690 |
| | standard | | Aldrich | |
| — | Internal | Nadolol | TargetMol | T1203 |
| | standard | | | |
| OATP1B1 | Inhibitor | Cyclosporin A | TCI | C2408 |
| | Substrate | Estrone 3-sulfate | WuXi | 20191126 |
| | | | AppTec | |
| OATP1B3 | Inhibitor | Rifampicin | Sigma- | R3501 |
| | | | Aldrich | |
| | Substrate | β-Estradiol 17- | Cayman | 16156 |
| | | (β-D-glucuronide) | chemical | |

HEK-293 cells stably expressing the human OATP1B1 and OATP1B3 transporter and the HEK293-MOCK cell line were licensed from GenoMembrane (Kanagawa, Japan). The culture medium was DMEM (HEK293-OATP1B3 cells were maintained in DMEM/F12) supplemented with 10.0% FBS, 500 μg/mL G418 sulfate solution, 100 U/mL penicillin-G and 100 μg/mL streptomycin. Cells were incubated in 5.0% CO₂ at 37.0° C. with saturated humidity. HEK293-OATP1B1 (Passage: 19), OATP1B3 (Passage: 19), and HEK293-MOCK (Passage: 14 and 16) were grown to 80.0 to 90.0% confluence in the culture flask. Trypsin/EDTA (0.05%/0.02%, w/v) was added to detach the cells from the flask. The cells were seeded onto 96-well plates with a density of 5.00×10⁴ cells/well, and then incubated for 24 hours in 5.0% CO₂ at 37.0° C. with saturated humidity before being used in the uptake study.

Pre-incubation: Cell culture medium was removed from the 96-well plates seeded with either HEK293-OATP1B1 and OATP1B3 or HEK293-MOCK cells, followed by rinsing the cells twice with the warm (37.0° C.) Transport Buffer. The cells were then pre-incubated for 30 min with the Transport Buffer in the Presence and Absence of positive inhibitors in 5.0% CO₂ at 37.0° C. with saturated humidity.

Uptake incubation: At the end of the pre-incubation, the buffers were removed and the cells were treated with Compound I.6 or Compound I.2 at 0.100 or 1.00 μM in the presence and absence of positive inhibitor. In separate wells, the marker substrate for transporter cells were incubated with dosing solution (Table A), all treatments were conducted in triplicate wells in 5.0% CO₂ at 37.0° C. with saturated humidity.

TABLE A
The information of inhibitor and substrate of individual transporter

| | | | Concen- | Dosing |
|---|---|---|---|---|
| Transporter | Application | Compound ID | trations | time |
| OATP1B1 | Inhibitor | cyclosporin A | 10.0 μM | 0.5 min |
| | Substrate | estrone 3-sulfate | 60.0 nM | |
| OATP1B3 | Inhibitor | rifampicin | 30.0 μM | 5 min |
| | Substrate | β-estradiol 17- | 5.00 μM | |
| | | (β-D-glucuronide) | | |

Cell lysing: At the end of the incubations, dosing solution was removed. After removing the remaining dosing solution, the cell was rinsed three times with ice-cold Transport Buffer (2.0-8.0° C.) followed by adding 100 μL cold acetonitrile:methanol (95: 5, v: v) containing internal standard into the cells treated with test compound for bioanalysis. For the cells dosed with positive control, the cells were added 100 μL cold acetonitrile:methanol (95:5, v: v) containing internal standard for bioanalysis. All samples were gently shaken for 30 min. Then for test compound and positive control, 75 μL of the cell lysate was mixed with 75 μL of Transport Buffer and 150 μL of cold acetonitrile:methanol (95:5, v:v) containing internal standard. The lysis sample was centrifuged for 10 min at 3220×g before the supernatant, defined as the terminal sample, was taken for the determination of the intracellular uptake of the test compounds or substrate by LC-MS/MS.

Uptake Fold was calculated using equation (1)

$$Uptake Fold = \frac{Transport Activity of Transporter cells}{Transport Activity of HEK 293 - MOCK cells} \qquad ( 1 )$$

TABLE 4
Uptake ratio of select compounds of Formula
(I) in uptake transporter assay:

| | Test | | | |
|---|---|---|---|---|
| Transporter | Concentration | | I.2 | I.6 |
| OATP1B1 | 0.1 | μM | 2.87 | 11.5 |
| | 1 | μM | 1.10 | 18.2 |
| OATP1B3 | 0.1 | μM | 2.23 | 1.74 |
| | 1 | μM | 2.34 | 4.9 |

### In Vivo Pharmacokinetic Studies

The compounds of this invention were tested orally administered in a formulation of 25% PEG 400: 10% solutol: 65% water with a 5 mL/Kg solution across species of CD-1 mice, C57BL/J6 mice, or Sprague Dawley rats following IACUC guidelines. The rat PK of Compound I.9 and Compound I.10 PO dose of 32 mpk exemplifies the methodology used across species.

The purpose of this rat PK study was to determine the pharmacokinetics of Compound I.9, Compound I.6 and Compound I.10 in plasma following oral gavage administration of Compound I.9 and Compound I.10 to male SD rats. For PO groups, the liver and plasma concentration were measured at 1, 2, 6 and 10 h post-dose. Concentrations of Compound I.9, Compound I.6 and Compound I.10 in plasma and tissue samples were determined by a liquid chromatography tandem mass spectrometry (LC-MS/MS) method. Oral (PO) Dose—Groups 1 and 2 Compound I.9 was prepared as a clear solution in 25% PEG400+10% Solutol+65% water (Adjust the final pH to 7˜8). Groups 3 and 4 Compound I.10 was prepared as a homogeneous suspension in 25% PEG400+10% Solutol+65% water (Adjust the final pH to 7˜8). Blood (about 0.2 mL) was collected at each time point via jugular vein puncture from each study animal into a tube A. The following 4-mix stabilizer for esterase inhibition was added into a pre-chilled commercial tube B containing potassium (K2) EDTA (0.85-1.15 mg) in advance. Then the blood in the tube A will be accurately transferred (4-mix stabilizer: blood, 1:10 (v:v)) into the tube B and placed on wet ice until centrifugation.

| | Stock | | Volume | Conc | Cocktail to Blood | Final Conc in |
|---|---|---|---|---|---|---|
| Inhibitor | Concentration | Solvent | (mL) | (mM) | sample ratio | matrix/sample |
| Citric Acid | 600 mM | Water | 0.5 | 150 mM | 1:10 (v:v), Eg. | 15 mM |
| PMSF | 400 mM | DMSO | 0.5 | 100 mM | 0.1 mL cocktail + | 10 mM |
| NaF | 400 mM | Water | 0.5 | 100 mM | 1 mL blood | 10 mM |
| Dichlorvos | 400 mM | Water | 0.5 | 100 mM | | 10 mM |

### Tissue Processing

The following 4-mix stabilizer was added into the cold homogenizing buffer (MeOH/15 mM PBS 1:2) in advance. Then each tissue was weighed and added the cold homogenizing buffer with stabilizer at the ratio of 1:9 (1 g tissue with 9 mL buffer), then homogenized on wet ice. The tissue homogenate was kept at −60° C. or lower until LC-MS/MS analysis.

| | Stock | | Volume | Conc | Cocktail to Blood | Final Conc in |
|---|---|---|---|---|---|---|
| Inhibitor | Concentration | Solvent | (mL) | (mM) | sample ratio | matrix/sample |
| Citric Acid | 600 mM | Water | 0.5 | 30 mM | 1:10 (v:v), Eg. | 3 mM |
| PMSF | 400 mM | DMSO | 0.5 | 20 mM | 0.1 mL cocktail + | 2 mM |
| NaF | 400 mM | Water | 0.5 | 20 mM | 1 mL (MeOH/15 | 2 mM |
| Dichlorvos | 400 mM | Water | 0.5 | 20 mM | mM PBS 1:2) | 2 mM |
| Water | NA | NA | 8 | NA | | NA |

Bioanalytical Analysis The concentrations of Compound I.9, Compound I.6 and Compound I.10 in plasma and tissue were determined by using an LC-MS/MS method. Instrumentation and conditions for

Compound I.9, Compound I.10 and Compound I.6 in plasma and tissue homogenate.
LC parameters
Equipment: ACQUITY UPLC System
Analytical column: ACQUITY UPLC HSS T3 1.8 μm 2.1×50 mm
Inject volume: 5 μL for plasma; 3.5 μL for tissue homogenate
Mobile phase A: 0.1% FA & 2 mmol/L HCOONH₄ in water/ACN (v/v, 95:5)
Mobile phase B: 0.1% FA & 2 mmol/L HCOONH₄ in ACN/water (v/v, 95:5)
Elution Mode: Gradient

Compound I.9, Compound I.10 and Compound I.6 in Plasma and Tissue Homogenate

| | Flow Rate | | |
|---|---|---|---|
| Time (min) | (mL/min) | A (%) | B (%) |
| Initial | 0.650 | 63 | 37 |
| 1.30 | 0.650 | 25 | 75 |
| 1.80 | 0.650 | 0 | 100 |
| 2.00 | 0.650 | 0 | 100 |
| 2.01 | 0.650 | 63 | 37 |
| 2.20 | 0.650 | 63 | 37 |

Mass spectrometer: Triple Quad 6500 plus
Ionization mode: ESI (+)
Detective mode: MRM

Following single PO 1 and PO 2 administration of Compound I.9 at 32 mg/kg in male SD rats, the PK parameters of Compound I.9 in plasma and tissue were not determined due to most concentrations were below the lower limit of quantitation. Following single PO 3 and PO 4 administration of Compound I.10, respectively, at 32 mg/kg in male SD rats, the PK parameters of Compound I.10 in plasma and tissue were not determined due to most concentrations were below the lower limit of quantitation.

TABLE 5
Compounds of Formula I ability to preferentially
deliver HBV S-antigen inhibitor to liver over
plasma (Sprague-Dawley rats, n = 3 animals):

| | | | | | “Active |
|---|---|---|---|---|---|
| | | | Observed level of | | metabolite” |
| | | “Active | “active metabolite” | | AUC-based |
| | Dose, | metabolite” | AUC_{0→last}, ng · h/g | | Liver/Plasma |
| “Prodrug” | mpk | released | Plasma | Liver | ratio |
| I.9 | 32 | I.6 | 290 | 29367 | 101 |
| I.10 | 32 | I.6 | 146 | 19444 | 133 |

TABLE 6
Compounds of Formula I ability to preferentially deliver HBV S-antigen inhibitor
to liver over plasma (mouse, n = 3 animals, ND = not determined, if one
or more animals' plasma level of observed metabolite is BQL, below level of quantitation):

| | | | | | Observed level of active | | “Active |
|---|---|---|---|---|---|---|---|
| | | | | Time | metabolite | | metabolite” |
| | Mouse | Dose, | Active | point, | Plasma, | Liver, | Liver/Plasma |
| Prodrug | breed | mpk | metabolite | h | ng/mL | ng/g | ratio |
| I.3 | CD-1 | 16 | I.2 | 2 | ND* | 186 ± 60.5 | ND |
| I.5 | CD-1 | 16 | I.2 | 2 | ND* | 343 ± 30.9 | ND |
| I.7 | CD-1 | 16 | I.6 | 2 | ND* | 193 ± 49 | ND |
| I.8 | C57BL/6 | 32 | I.6 | 1 | 51.7 ± 29.7 | 1106 ± 659 | 22.4 ± 13.8 |
| I.9 | CD-1 | 16 | I.6 | 2 | 4.0 ± 1.0 | 423 ± 335 | 97 ± 51 |
| I.9 | C57BL/6 | 32 | I.6 | 1 | 196 ± 83.4 | 6560 ± 1769 | 35.7 ± 8.0 |
| I.10 | CD-1 | 16 | I.6 | 1 | 15.4 ± 7.3 | 659 ± 149 | 162 |
| I.10 | C57BL/6 | 32 | I.6 | 1 | 9.6 | 2622 ± 2483 | 332 |
| I.11 | C57BL/6 | 32 | I.6 | 1 | 22.0 ± 11.3 | 811 ± 571 | 36.0 ± 20.9 |
| I.12 | C57BL/6 | 32 | I.6 | 1 | 69.3 ± 30.8 | 1831 ± 1296 | 24.5 ± 6.5 |
| I.14 | CD-1 | 16 | I.13 | 2 | ND* | 533 ± 169 | ND |

*Based on a limit of detection of 1 ng/mL in plasma, the compounds I.3, I.5, I.7, and I.14 have promising liver to plasma ratios of >186, >343, >193, and >533.

### In Vivo Efficacy

The purpose of this study was to investigate in vivo pharmacologic efficacy of Compound I.9 and Compound I.10 in an adeno-associated virus-hepatitis B virus (AAV-HBV) transfected mouse model. Mice were injected through tail vein with 1×10¹¹ vector genomes of recombinant AAV-HBV in 200 μL of phosphate-buffered saline per mouse on Predose Day 0. Mice were bled to prepare 10 μL of serum per mouse on Predose Days 21 and 28 (21 and 28 days after AAV-HBV injection). The serum samples were stored at −70° C. and transferred to Clinical Pathology Department for HBsAg quantitative detections (as baseline). Based on body weight and serum levels of HBsAg on Predose Day 35, 48 mice were selected randomized into seven groups, with six mice per group.

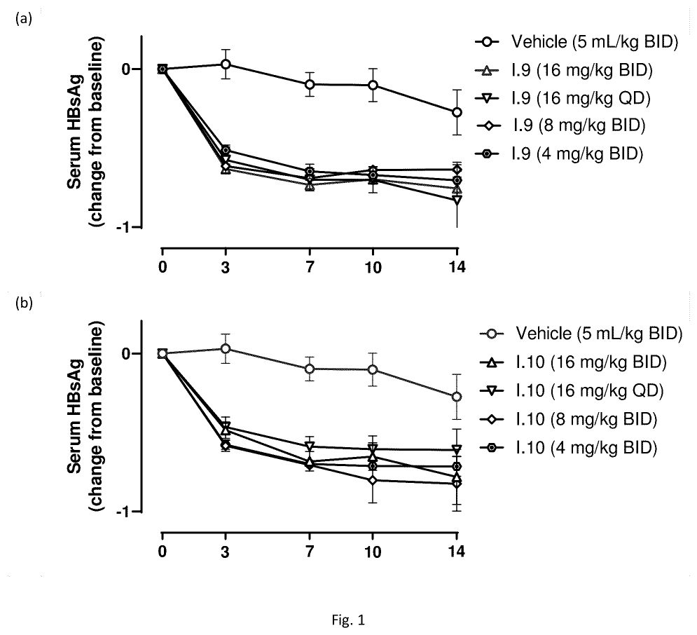

Vehicle was administered twice daily with a 12-hour interval during Day 0-13. I.10 or I.9 was administered at either 16 mg/kg/dose, 8 mg/kg/dose and 4 mg/kg/dose twice daily or 16 mg/kg/dose once daily. In the case of twice-daily regimen, doses were administered with a 12-hour interval during Day 0-13 and once on Day 14. All the test articles were administered by oral gavage at 5 mL/kg/dose. Animal clinical signs were monitored once daily, and body weight was measured twice weekly during Day 0-14. Mice were bled to prepare 10 μL of serum per mouse on Days 0, 3, 7, 10 and 14. The serum samples were stored at −70° C. and transferred to Clinical Pathology Department for viral marker detection. HBsAg was detected on Days 0, 3, 7, 10 and 14. For the groups administered with Compound I.10 or Compound I.9, the 1^{st} three mice in each group were bled at 0.5 and 4 hr, and the 2^{nd} three mice in each group were bled at 1 hr and 8 hr, after the 1^{st} dose on Day 0. Plasma samples of 7 μL per mouse per time point were prepared, stored at −70° C. and transferred to Metabolism Department for bioanalysis (data report was separately sent to sponsor by Labcorp Metabolism Department). The mice in vehicle control group were sacrificed without blood or tissue collection on Day 14. The 1^{st} three mice and the 2^{nd} three mice in the groups treated with I.10 or I.9 were sacrificed at 2 hr and 6 hr, respectively, after dosing on Day 14. For these mice, livers were harvested after local perfusion with normal saline. Livers were taken out from abdominal cavity, rinsed with saline and placed on a soft absorbable paper to drain out all remaining liquid. Livers were weighed, cut into small pieces, put into tubes and snap frozen in liquid nitrogen. The liver pieces were homogenized with methanol solution (MeOH:15 mM PBS=1:2) at a ratio of 1:9 (1 g tissue with 9 mL buffer) in the ice-cold condition. The homogenates were stored at −70° C. and transferred to Metabolism Department for bioanalysis (data report was separately sent to sponsor by Labcorp Metabolism Department). Serum hepatitis B surface antigen (HBsAg) was measured using ARCHITECT i2000 (Abbott Laboratories, Lake Bluff, IL, USA) with supporting reagents. Compared with the vehicle control group, serum HBsAg level of the groups treated with Compound I.9 and Compound I.10 decreased by 0.42-0.69 Log₁₀, units after two weeks of treatment following doses of 64 MPK BID, 32 MPK BID, and 16 MPK BID maximal efficacy was observed (not plotted). Based on the initial maximum efficacy at high doses, Compounds I.9 and I.10 were dosed at 16 MPK BID, 16 MPK QD, 8 MPK BID, and 4 MPK BID with results as shown in FIG. 1.

GLOSSARY OF ABBREVIATIONS

| | |
|---|---|
| AUC | The area under the plasma or tissue concentration- |
| | time curve |
| AUC₀₋ₗₐₛₜ | The area under the plasma or tissue concentration- |
| | time curve from time zero to the last quantifiable |
| | concentration |
| AUC_{0-inf} | The area under the plasma or tissue concentration- |
| | time curve from time zero extrapolated to infinity |
| | were calculated using the linear/log trapezoidal rule |
| BQL | Below the quantifiable limit |
| Cₘₐₓ | Peak plasma or tissue concentration |
| C₀ | Initial plasma or tissue concentration |
| Conc. | Concentration |
| Cl | Total body clearance |
| ECL | Electrochemiluminescence |
| GLP | Good laboratory practice |
| h | Hour (s) |
| HPLC-UV | High-performance liquid chromatography/ultraviolet |
| IV | Intravenous |
| K₂-EDTA | Ethylene diaminetetra acetic acid dipotassium salt |
| LC-MS/MS | Liquid chromatography tandem mass spectrometry |
| LLOQ | Lower limit of quantitation |
| min | Minute (s) |
| MRT | Mean residence time |
| MRT₀₋ₗₐₛₜ | Mean residence time from time zero to the last |
| | quantifiable concentration |
| MRT0-inf | Mean residence time from time zero to infinity |
| PK | Pharmacokinetic |
| PEG | Polyethylene glycol |
| RT | Room temperature |
| SOP | Standard operating procedure |
| Tmax | Time at which the Cmax is achieved |
| T½ | Terminal half-life |
| Vdss | Volume of distribution at steady state |

1. A compound of Formula I below: 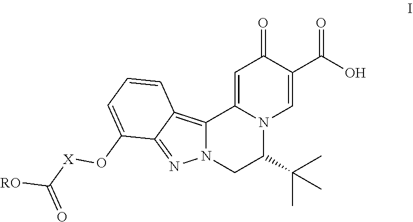
wherein R is hydrogen, ethyl, or a linear, cyclic, or branched C₃-C₈ alkyl group, and X is a linear, cyclic, or branched C₅-C₁₀ alkylene group;
or a pharmaceutically acceptable salt thereof.

2. The compound, or a pharmaceutically acceptable salt thereof, of claim 1, wherein X is a linear C₅-C₈ alkylene group.

3. The compound of claim 1, or a pharmaceutically acceptable salt thereof, wherein X is selected from the group consisting of a linear —C₅H₁₀—, linear —C₆H₁₂—, and linear —C₇H₁₄—.

4. The compound of claim 1, or a pharmaceutically acceptable salt thereof, wherein R is selected from the group consisting of n-C₂H₅, n-C₃H₇, i-C₃H₇, n-C₄H₉, n-C₅H₁₁, n-C₆H₁₃, 2-ethylbutyl, n-C₇H₁₅, and n-C₈H₁₇.

5. The compound of claim 1, or a pharmaceutically acceptable salt thereof, wherein R is H.

6. The compound of claim 1, or a pharmaceutically acceptable salt thereof, wherein R is i-C₃H₇.

7. The compound of claim 1, or a pharmaceutically acceptable salt thereof, wherein R is n-C₄H₉.

8. The compound of claim 1, or a pharmaceutically acceptable salt thereof, selected from the group consisting of: 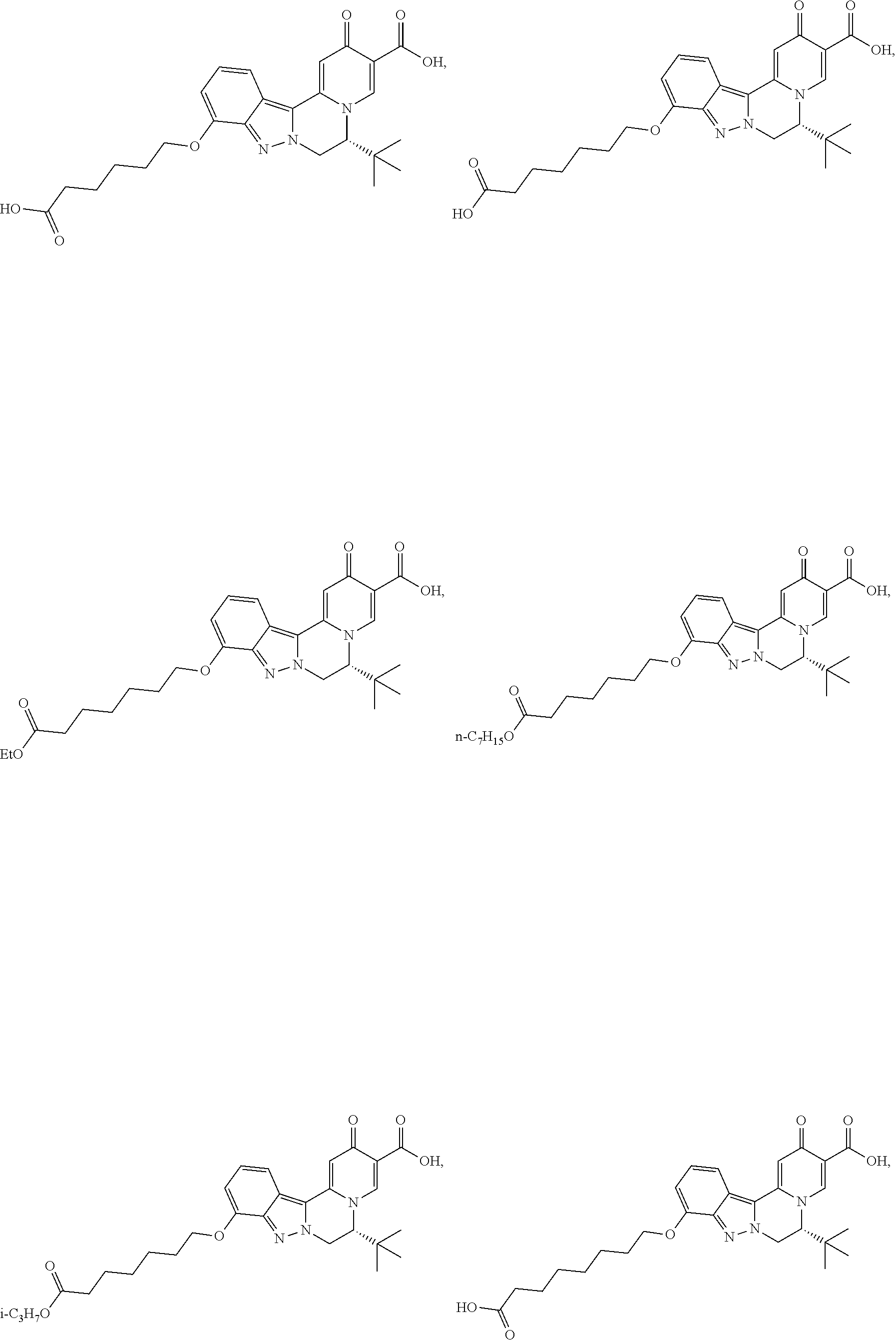 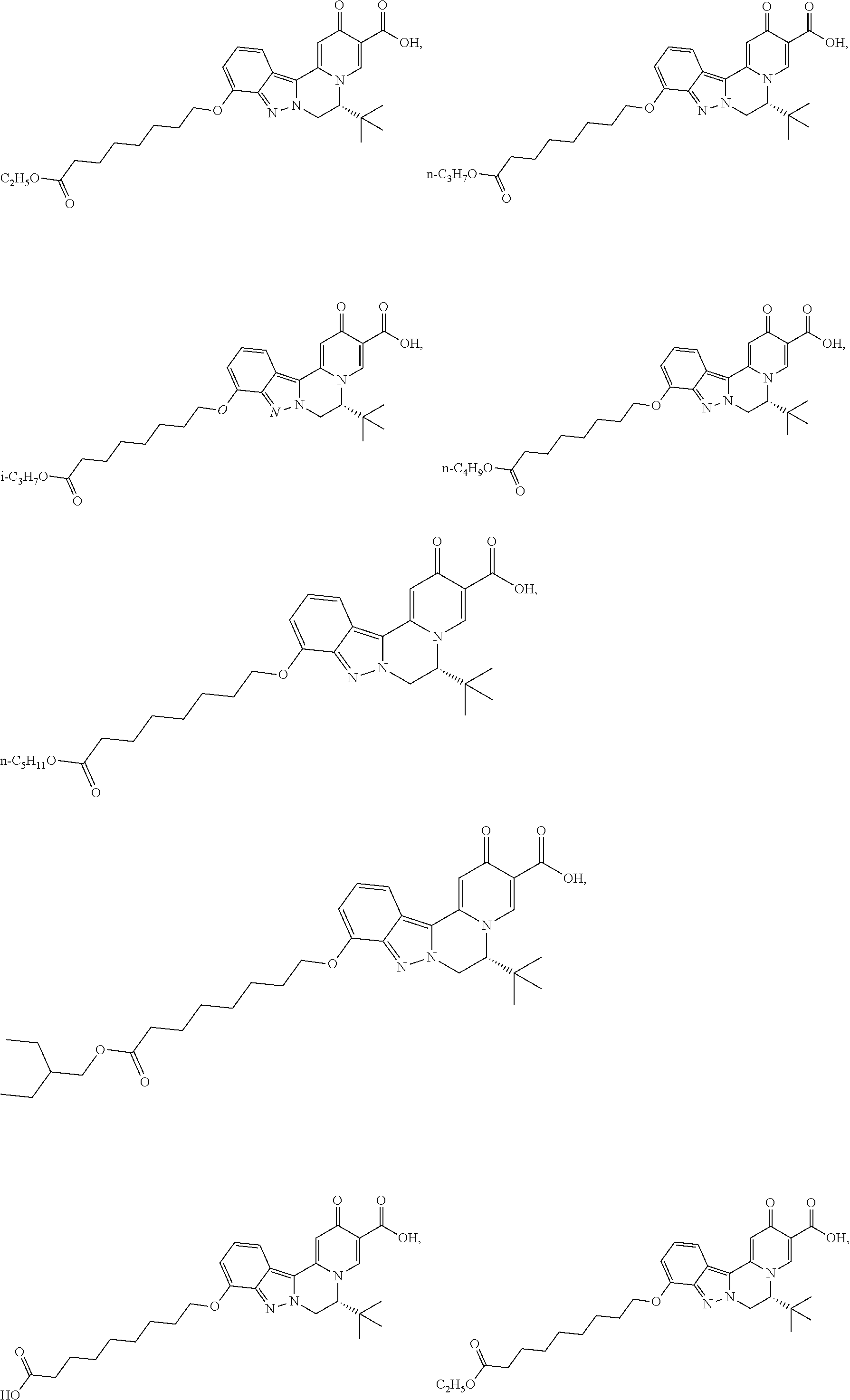
and a pharmaceutically acceptable salt of each of the foregoing.

9. A compound having the following structure:

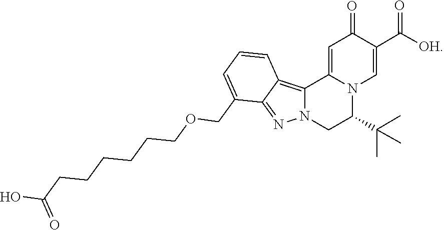 [SMILES: CC(C)(C)[C@@H]1Cn2nc3c(COCCCCCCC(=O)O)cccc3c2-c2cc(=O)c(C(=O)O)cn21]

10. A compound having the following structure:

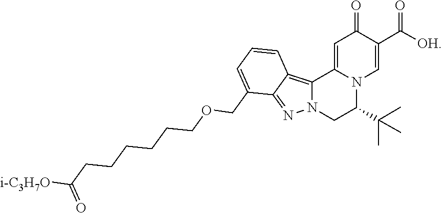

11. A compound having the following structure:

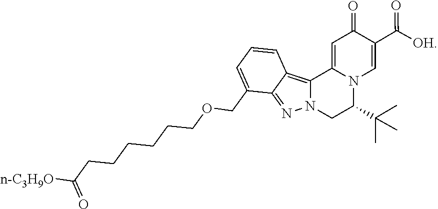

12. A pharmaceutical composition comprising a compound of claim 1, or a pharmaceutically acceptable salt thereof, and a pharmaceutically acceptable carrier.

13. A method of treating human hepatitis B in a human subject, comprising administering to the subject a pharmaceutical composition of claim 12.

14. The method of claim 13, further comprising: administering to the subject an additional therapeutic agent selected from the group consisting of an HBV replication inhibitor, an HBsAg targeting agent; and an immunomodulator.

15. A method of treating human hepatitis D in a human subject, comprising administering to the subject a pharmaceutical composition of claim 12.

16. The method of claim 15, further comprising: administering to the subject an additional therapeutic agent selected from the group consisting of a DNA polymerase inhibitor, an HBV capsid inhibitor, an HBV-targeting antibody, and an HBV-targeting therapeutic vaccine.

17. A method to inhibit replication of hepatitis B virus, comprising contacting the hepatitis B virus, either in vitro or in vivo, with a compound, or a pharmaceutically acceptable salt thereof, according to claim 1.

18. A method to inhibit replication of hepatitis D virus, comprising contacting the hepatitis B virus, either in vitro or in vivo, with a compound, or a pharmaceutically acceptable salt thereof, according to claim 1.

19-21. (canceled)
